SEQUENCE LISTING (146 sequences)

SEQ ID NO: 1 - DNA, 26 bp, synthetic construct
  source 1..26: mol_type = other DNA
ttctaatacgactcactatagggatg

SEQ ID NO: 2 - DNA, 32 bp, synthetic construct
  source 1..32: mol_type = other DNA
catcccctacatccacatactaattaaccttc

SEQ ID NO: 3 - DNA, 32 bp, synthetic construct
  source 1..32: mol_type = other DNA
catcccctactttcacttcacaacatcacttc

SEQ ID NO: 4 - DNA, 36 bp, synthetic construct
  source 1..36: mol_type = other DNA
catcccgacatacagattaaccagacagtgaccttc

SEQ ID NO: 5 - DNA, 28 bp, synthetic construct
  source 1..28: mol_type = other DNA
catcccctacatccacatactaattaac

SEQ ID NO: 6 - DNA, 28 bp, synthetic construct
  source 1..28: mol_type = other DNA
catccctaccatcacattcaataatcct

SEQ ID NO: 7 - DNA, 82 bp, synthetic construct
  source 1..82: mol_type = other DNA
gaaggttaattagtatgtggatgtaggggatgtggggttggttgtgttgggtgttgtgtcatccctatagtgagtcgtatta

SEQ ID NO: 8 - DNA, 84 bp, synthetic construct
  source 1..84: mol_type = other DNA
gaaggttaattagtatgtggatgtaggggatgagtccgtggtagggcaggttggggtgactcatccctatagtgagtcgtatta

SEQ ID NO: 9 - DNA, 87 bp, synthetic construct
  source 1..87: mol_type = other DNA
gaaggttaattagtatgtggatgtaggggatgtggtggcaggaggactatttatttgcttttctcatccctatagtgagtcgtatta

SEQ ID NO: 10 - DNA, 80 bp, synthetic construct
  source 1..80: mol_type = other DNA
gaaggttaattagtatgtggatgtaggggatgtggtggatggcgcagtcggcgacaacatccctatagtgagtcgtatta

SEQ ID NO: 11 - DNA, 79 bp, synthetic construct
  source 1..79: mol_type = other DNA
gttaattagtatgtggatgtaggggatgtgttagggggttggttttgggttggcatccctatagtgagtcgtattagaa

SEQ ID NO: 12 - DNA, 78 bp, synthetic construct
  source 1..78: mol_type = other DNA
gttaattagtatgtggatgtaggggatgttcttcttcttcttcttcttcttccatccctatagtgagtcgtattagaa

SEQ ID NO: 13 - DNA, 83 bp, synthetic construct
  source 1..83: mol_type = other DNA
gttaattagtatgtggatgtaggggatgagtccgtggtagggcaggttggggtgactcatccctatagtgagtcgtattagaa

SEQ ID NO: 14 - DNA, 86 bp, synthetic construct
  source 1..86: mol_type = other DNA
gttaattagtatgtggatgtaggggatgtggtggcaggaggactatttatttgcttttctcatccctatagtgagtcgtattagaa

SEQ ID NO: 15 - DNA, 79 bp, synthetic construct
  source 1..79: mol_type = other DNA
gttaattagtatgtggatgtaggggatgtggtggatggcgcagtcggcgacaacatccctatagtgagtcgtattagaa

SEQ ID NO: 16 - DNA, 79 bp, synthetic construct
  source 1..79: mol_type = other DNA
tgatgttgtgaagtgaaagtaggggatgtgttagggggttggttttgggttggcatccctatagtgagtcgtattagaa

SEQ ID NO: 17 - DNA, 79 bp, synthetic construct
  source 1..79: mol_type = other DNA
aggattattgaatgtgatggtagggatgtgttagggggttggttttgggttggcatccctatagtgagtcgtattagaa

SEQ ID NO: 18 - DNA, 75 bp, synthetic construct
  source 1..75: mol_type = other DNA
gttaattagtatgtggatgtaggggatgtgggggttgaggctaagccgacatccctatagtgagtcgtattagaa

SEQ ID NO: 19 - DNA, 79 bp, synthetic construct
  source 1..79: mol_type = other DNA
gttaattagtatgtggatgtaggggatgtgtgggggtggactgggtgggtacccatccctatagtgagtcgtattagaa

SEQ ID NO: 20 - RNA, 65 nt, synthetic construct
  source 1..65: mol_type = other RNA
gggatgacacaacacccaacacaaccaaccccacatcccctacatccacatactaattaaccttc

SEQ ID NO: 21 - RNA, 67 nt, synthetic construct
  source 1..67: mol_type = other RNA
gggatgagtcaccccaacctgccctaccacggactcatcccctacatccacatactaattaaccttc

SEQ ID NO: 22 - RNA, 70 nt, synthetic construct
  source 1..70: mol_type = other RNA
gggatgagaaaagcaaataaatagtcctcctgccaccacatcccctacatccacatactaattaaccttc

SEQ ID NO: 23 - RNA, 63 nt, synthetic construct
  source 1..63: mol_type = other RNA
gggatgttgtcgccgactgcgccatccaccacatcccctacatccacatactaattaaccttc

SEQ ID NO: 24 - DNA, 16 bp, synthetic construct
  source 1..16: mol_type = other DNA
ctacatccacatacta

SEQ ID NO: 25 - DNA, 22 bp, synthetic construct
  source 1..22: mol_type = other DNA
gttaattagtatgtggatgtag

SEQ ID NO: 26 - DNA, 21 bp, synthetic construct
  source 1..21: mol_type = other DNA
gacatacagattaaccagaca

SEQ ID NO: 27 - DNA, 26 bp, synthetic construct
  source 1..26: mol_type = other DNA
gtcactgtctggttaatctgtatgtc

SEQ ID NO: 28 - DNA, 17 bp, synthetic construct
  source 1..17: mol_type = other DNA
ctactttcacttcacaa

SEQ ID NO: 29 - DNA, 22 bp, synthetic construct
  source 1..22: mol_type = other DNA
tgatgttgtgaagtgaaagtag

SEQ ID NO: 30 - DNA, 16 bp, synthetic construct
  source 1..16: mol_type = other DNA
taccatcacattcaat

SEQ ID NO: 31 - DNA, 22 bp, synthetic construct
  source 1..22: mol_type = other DNA
aggattattgaatgtgatggta

SEQ ID NO: 32 - DNA, 41 bp, synthetic construct
  source 1..41: mol_type = other DNA
catccctgcacgccaaaccgtggcgacgtaatagagctgga

SEQ ID NO: 33 - DNA, 94 bp, synthetic construct
  source 1..94: mol_type = other DNA
tccagctctattacgtcgccacggtttggcgtgcagggatgtggggttggttgtgttgggtgttgtgtcatccctatagtgagtcgtattagaa

SEQ ID NO: 34 - RNA, 74 nt, synthetic construct
  source 1..74: mol_type = other RNA
gggatgacacaacacccaacacaaccaaccccacatccctgcacgccaaaccgtggcgacgtaatagagctgga

SEQ ID NO: 35 - DNA, 41 bp, synthetic construct
  source 1..41: mol_type = other DNA
catcccttcaggtcaataagtgaccaagtaatagtgggatg

SEQ ID NO: 36 - DNA, 94 bp, synthetic construct
  source 1..94: mol_type = other DNA
catcccactattacttggtcacttattgacctgaagggatgtggggttggttgtgttgggtgttgtgtcatccctatagtgagtcgtattagaa

SEQ ID NO: 37 - RNA, 74 nt, synthetic construct
  source 1..74: mol_type = other RNA
gggatgacacaacacccaacacaaccaaccccacatcccttcaggtcaataagtgaccaagtaatagtgggatg

SEQ ID NO: 38 - DNA, 32 bp, synthetic construct
  source 1..32: mol_type = other DNA
tccagtattacgtcgccacggtttggcgtgcc

SEQ ID NO: 39 - DNA, 80 bp, synthetic construct
  source 1..80: mol_type = other DNA
ggcacgccaaaccgtggcgacgtaatacgactcactatagggagaaaatctcccgacatacagattaaccagacagtgac

SEQ ID NO: 40 - DNA, 53 bp, synthetic construct
  source 1..53: mol_type = other DNA
gtcactgtctggttaatctgtatgtcgggagattttctccctatagtgagtcg

SEQ ID NO: 41 - RNA, 41 nt, synthetic construct
  source 1..41: mol_type = other RNA
gggagaaaatctcccgacatacagattaaccagacagtgac

SEQ ID NO: 42 - DNA, 38 bp, synthetic construct
  source 1..38: mol_type = other DNA
gagtaggtcgtcgccacggtttggcgtgcatggaagga

SEQ ID NO: 43 - DNA, 32 bp, synthetic construct; identical to SEQ ID NO: 38
  source 1..32: mol_type = other DNA

SEQ ID NO: 44 - DNA, 80 bp, synthetic construct; identical to SEQ ID NO: 39
  source 1..80: mol_type = other DNA

SEQ ID NO: 45 - DNA, 53 bp, synthetic construct; identical to SEQ ID NO: 40
  source 1..53: mol_type = other DNA

SEQ ID NO: 46 - RNA, 41 nt, synthetic construct; identical to SEQ ID NO: 41
  source 1..41: mol_type = other RNA

SEQ ID NO: 47 - DNA, 26 bp, synthetic construct
  source 1..26: mol_type = other DNA
ttctaatacgactcactatagggatg

SEQ ID NO: 48 - DNA, 32 bp, synthetic construct; identical to SEQ ID NO: 2
  source 1..32: mol_type = other DNA

SEQ ID NO: 49 - DNA, 77 bp, synthetic construct
  source 1..77: mol_type = other DNA
atccacatactaattaacgggatgtggggttggttgtgttgggtgttgtgtcatccctatagtgagtcgtattagaa

SEQ ID NO: 50 - DNA, 84 bp, synthetic construct
  source 1..84: mol_type = other DNA
gttaattagtatgtggatgtaggggatggacaaggaaaatccttcaatgaagtgggtccatccctatagtgagtcgtattagaa

SEQ ID NO: 51 - DNA, 24 bp, synthetic construct
  source 1..24: mol_type = other DNA
catcccgttaattagtatgtggat

SEQ ID NO: 52 - DNA, 75 bp, synthetic construct
  source 1..75: mol_type = other DNA
atccacatactaattaacgggatgtgttagggggttggttttgggttggcatccctatagtgagtcgtattagaa

SEQ ID NO: 53 - DNA, 44 bp, synthetic construct
  source 1..44: mol_type = other DNA
catccctccttccatgaacgccaaaccgtggcgacgacctactc

SEQ ID NO: 54 - DNA, 99 bp, synthetic construct
  source 1..99: mol_type = other DNA
gagtaggtcgtcgccacggtttggcgttcatggaaggagggatgagtccgtggtagggcaggttggggtgactcatccctatagtgagtcgtattagaa

SEQ ID NO: 55 - DNA, 32 bp, synthetic construct
  source 1..32: mol_type = other DNA
catcccaaagagtaggtcgtttcatggaagga

SEQ ID NO: 56 - DNA, 83 bp, synthetic construct
  source 1..83: mol_type = other DNA
tccttccatgaaacgacctactctttgggatgtgttagggggttggttttgggttggcatccctatagtgagtcgtattagaa

SEQ ID NO: 57 - DNA, 89 bp, synthetic construct
  source 1..89: mol_type = other DNA
tccttccatgcacgccaaaccgtggcgacgtaatacgactcactatagggagattcgtctcccgacatacagattaaccagacagtgac

SEQ ID NO: 58 - DNA, 35 bp, synthetic construct
  source 1..35: mol_type = other DNA
tccagctctattacgtcgccacggtttggcgtgca

SEQ ID NO: 59 - DNA, 54 bp, synthetic construct
  source 1..54: mol_type = other DNA
gtcactgtctggttaatctgtatgtcgggagacgaatctccctatagtgagtcg

SEQ ID NO: 60 - DNA, 32 bp, synthetic construct
  source 1..32: mol_type = other DNA
catcccgacatacagattaaccagacagtgac

SEQ ID NO: 61 - DNA, 87 bp, synthetic construct
  source 1..87: mol_type = other DNA
gtcactgtctggttaatctgtatgtcgggatgagtccgtggtagggcaggttggggtgactcatccctatagtgagtcgtattagaa

SEQ ID NO: 62 - DNA, 28 bp, synthetic construct
  source 1..28: mol_type = other DNA
catcccgtcactgtctggttaatctgta

SEQ ID NO: 63 - DNA, 79 bp, synthetic construct
  source 1..79: mol_type = other DNA
tacagattaaccagacagtgacgggatgtgttagggggttggttttgggttggcatccctatagtgagtcgtattagaa

SEQ ID NO: 64 - DNA, 22 bp, synthetic construct
  source 1..22: mol_type = other DNA
ttctaatacgactcactatagg

SEQ ID NO: 65 - DNA, 24 bp, synthetic construct
  source 1..24: mol_type = other DNA
ttctaatacgactcactataggga

SEQ ID NO: 66 - DNA, 26 bp, synthetic construct
  source 1..26: mol_type = other DNA
ttctaatacgactcactatagggatg

SEQ ID NO: 67 - DNA, 30 bp, synthetic construct
  source 1..30: mol_type = other DNA
ttctaatacgactcactatagggagggatg

SEQ ID NO: 68 - DNA, 34 bp, synthetic construct
  source 1..34: mol_type = other DNA
ttctaatacgactcactatagggagattgggatg

SEQ ID NO: 69 - DNA, 38 bp, synthetic construct
  source 1..38: mol_type = other DNA
ttctaatacgactcactatagggagattcgtcgggatg

SEQ ID NO: 70 - DNA, 42 bp, synthetic construct
  source 1..42: mol_type = other DNA
ttctaatacgactcactatagggagattcgtctcccgggatg

SEQ ID NO: 71 - DNA, 27 bp, synthetic construct
  source 1..27: mol_type = other DNA
ttggcctacatccacatactaattaac

SEQ ID NO: 72 - DNA, 27 bp, synthetic construct
  source 1..27: mol_type = other DNA
ggtccctacatccacatactaattaac

SEQ ID NO: 73 - DNA, 27 bp, synthetic construct
  source 1..27: mol_type = other DNA
catccctacatccacatactaattaac

SEQ ID NO: 74 - DNA, 74 bp, synthetic construct
  source 1..74: mol_type = other DNA
gttaattagtatgtggatgtaggccaatgttagggggttggttttgggttggcctatagtgagtcgtattagaa

SEQ ID NO: 75 - DNA, 76 bp, synthetic construct
  source 1..76: mol_type = other DNA
gttaattagtatgtggatgtagggacctgttagggggttggttttgggttggtccctatagtgagtcgtattagaa

SEQ ID NO: 76 - DNA, 78 bp, synthetic construct
  source 1..78: mol_type = other DNA
gttaattagtatgtggatgtagggatgtgttagggggttggttttgggttggcatccctatagtgagtcgtattagaa

SEQ ID NO: 77 - DNA, 82 bp, synthetic construct
  source 1..82: mol_type = other DNA
gttaattagtatgtggatgtagggatgtgttagggggttggttttgggttggcatccctccctatagtgagtcgtattagaa

SEQ ID NO: 78 - DNA, 86 bp, synthetic construct
  source 1..86: mol_type = other DNA
gttaattagtatgtggatgtagggatgtgttagggggttggttttgggttggcatcccaatctccctatagtgagtcgtattagaa

SEQ ID NO: 79 - DNA, 90 bp, synthetic construct
  source 1..90: mol_type = other DNA
gttaattagtatgtggatgtagggatgtgttagggggttggttttgggttggcatcccgacgaatctccctatagtgagtcgtattagaa

SEQ ID NO: 80 - DNA, 94 bp, synthetic construct
  source 1..94: mol_type = other DNA
gttaattagtatgtggatgtagggatgtgttagggggttggttttgggttggcatcccgggagacgaatctccctatagtgagtcgtattagaa

SEQ ID NO: 81 - DNA, 10 bp, synthetic construct
  source 1..10: mol_type = other DNA
ccctacggtg

SEQ ID NO: 82 - DNA, 11 bp, synthetic construct
  source 1..11: mol_type = other DNA
caccgtagggg

SEQ ID NO: 83 - DNA, 12 bp, synthetic construct
  source 1..12: mol_type = other DNA
ccctacggtgtt

SEQ ID NO: 84 - DNA, 13 bp, synthetic construct
  source 1..13: mol_type = other DNA
aacaccgtagggg

SEQ ID NO: 85 - RNA, 28 nt, synthetic construct
  source 1..28: mol_type = other RNA
catcccctacatccacatactaattaac

SEQ ID NO: 86 - DNA, 25 bp, synthetic construct
  source 1..25: mol_type = other DNA
ggttgggttttggttgggggattgt

SEQ ID NO: 87 - DNA, 24 bp, synthetic construct
  source 1..24: mol_type = other DNA
cttcttcttcttcttcttcttctt

SEQ ID NO: 88 - RNA, 59 nt, synthetic construct
  source 1..59: mol_type = other RNA
gggatgccaacccaaaaccaaccccctaacacatcccctacatccacatactaattaac

SEQ ID NO: 89 - RNA, 53 nt, synthetic construct
  source 1..53: mol_type = other RNA
gggatgccaacccaaaaccaaccccctaacactacatccacatactaattaac

SEQ ID NO: 90 - DNA, 33 bp, synthetic construct
  source 1..33: mol_type = other DNA
ccggttgggttttggttgggggattgtaaccgg

SEQ ID NO: 91 - RNA, 33 nt, synthetic construct
  source 1..33: mol_type = other RNA
ggccaacccaaaaccaaccccctaacattggcc

SEQ ID NO: 92 - DNA, 35 bp, synthetic construct
  source 1..35: mol_type = other DNA
ccctggttgggttttggttgggggattgtccaggg

SEQ ID NO: 93 - RNA, 35 nt, synthetic construct
  source 1..35: mol_type = other RNA
gggaccaacccaaaaccaaccccctaacaggtccc

SEQ ID NO: 94 - DNA, 37 bp, synthetic construct
  source 1..37: mol_type = other DNA
ccctacggttgggttttggttgggggattgtgtaggg

SEQ ID NO: 95 - RNA, 37 nt, synthetic construct
  source 1..37: mol_type = other RNA
gggatgccaacccaaaaccaaccccctaacacatccc

SEQ ID NO: 96 - DNA, 10 bp, synthetic construct
  source 1..10: mol_type = other DNA
gggagggatg

SEQ ID NO: 97 - DNA, 41 bp, synthetic construct
  source 1..41: mol_type = other DNA
ccctccctacggttgggttttggttgggggattgtgtaggg

SEQ ID NO: 98 - RNA, 41 nt, synthetic construct
  source 1..41: mol_type = other RNA
gggagggatgccaacccaaaaccaaccccctaacacatccc

SEQ ID NO: 99 - DNA, 14 bp, synthetic construct
  source 1..14: mol_type = other DNA
gggagattgggatg

SEQ ID NO: 100 - DNA, 45 bp, synthetic construct
  source 1..45: mol_type = other DNA
ccctctaaccctacggttgggttttggttgggggattgtgtaggg

SEQ ID NO: 101 - RNA, 45 nt, synthetic construct
  source 1..45: mol_type = other RNA
gggagattgggatgccaacccaaaaccaaccccctaacacatccc

SEQ ID NO: 102 - DNA, 18 bp, synthetic construct
  source 1..18: mol_type = other DNA
gggagattcgtcgggatg

SEQ ID NO: 103 - DNA, 49 bp, synthetic construct
  source 1..49: mol_type = other DNA
ccctctaagcagccctacggttgggttttggttgggggattgtgtaggg

SEQ ID NO: 104 - RNA, 49 nt, synthetic construct
  source 1..49: mol_type = other RNA
gggagattcgtcgggatgccaacccaaaaccaaccccctaacacatccc

SEQ ID NO: 105 - DNA, 22 bp, synthetic construct
  source 1..22: mol_type = other DNA
gggagattcgtctcccgggatg

SEQ ID NO: 106 - DNA, 53 bp, synthetic construct
  source 1..53: mol_type = other DNA
ccctctaagcagagggccctacggttgggttttggttgggggattgtgtaggg

SEQ ID NO: 107 - RNA, 53 nt, synthetic construct
  source 1..53: mol_type = other RNA
gggagattcgtctcccgggatgccaacccaaaaccaaccccctaacacatccc

SEQ ID NO: 108 - DNA, 29 bp, synthetic construct
  source 1..29: mol_type = other DNA
tcagtggggttggacgggatggtgcctga

SEQ ID NO: 109 - DNA, 32 bp, synthetic construct
  source 1..32: mol_type = other DNA
tcttttcgtttatttatcaggaggacggtggt

SEQ ID NO: 110 - DNA, 25 bp, synthetic construct
  source 1..25: mol_type = other DNA
aacagcggctgacgcggtaggtggt

SEQ ID NO: 111 - RNA, 63 nt, synthetic construct
  source 1..63: mol_type = other RNA
gggatgagtcaccccaacctgccctaccacggactcatcccctacatccacatactaattaac

SEQ ID NO: 112 - RNA, 66 nt, synthetic construct
  source 1..66: mol_type = other RNA
gggatgagaaaagcaaataaatagtcctcctgccaccacatcccctacatccacatactaattaac

SEQ ID NO: 113 - RNA, 59 nt, synthetic construct
  source 1..59: mol_type = other RNA
gggatgttgtcgccgactgcgccatccaccacatcccctacatccacatactaattaac

SEQ ID NO: 114 - DNA, 28 bp, synthetic construct
  source 1..28: mol_type = other DNA
catcccctacatccacatactaattaac

SEQ ID NO: 115 - DNA, 28 bp, synthetic construct
  source 1..28: mol_type = other DNA
gtaggggatgtaggtgtatgattaattg

SEQ ID NO: 116 - DNA, 32 bp, synthetic construct; identical to SEQ ID NO: 3
  source 1..32: mol_type = other DNA

SEQ ID NO: 117 - DNA, 28 bp, synthetic construct
  source 1..28: mol_type = other DNA
gtaggggatgaaagtgaagtgttgtagt

SEQ ID NO: 118 - DNA, 28 bp, synthetic construct
  source 1..28: mol_type = other DNA
catccctaccatcacattcaataatcct

SEQ ID NO: 119 - DNA, 28 bp, synthetic construct
  source 1..28: mol_type = other DNA
gtagggatggtagtgtaagttattagga

SEQ ID NO: 120 - RNA, 59 nt, synthetic construct
  source 1..59: mol_type = other RNA
gggatgccaacccaaaaccaaccccctaacacatccccatctttcacttcacaacatca

SEQ ID NO: 121 - RNA, 59 nt, synthetic construct
  source 1..59: mol_type = other RNA
gggatgccaacccaaaaccaaccccctaacacatccctaccatcacattcaataatcct

SEQ ID NO: 122 - DNA, 21 bp, synthetic construct
  source 1..21: mol_type = other DNA
agccgaatcggagttgggggt

SEQ ID NO: 123 - DNA, 25 bp, synthetic construct
  source 1..25: mol_type = other DNA
ccatgggtgggtcaggtgggggtgt

SEQ ID NO: 124 - RNA, 55 nt, synthetic construct
  source 1..55: mol_type = other RNA
gggatgtcggcttagcctcaacccccacatcccctacatccacatactaattaac

SEQ ID NO: 125 - RNA, 59 nt, synthetic construct
  source 1..59: mol_type = other RNA
gggatgggtacccacccagtccacccccacacatcccctacatccacatactaattaac

SEQ ID NO: 126 - RNA, 55 nt, synthetic construct
  source 1..55: mol_type = other RNA
gggatgccaacccaaaaccaaccccctaacacatcccgttaattagtatgtggat

SEQ ID NO: 127 - RNA, 64 nt, synthetic construct
  source 1..64: mol_type = other RNA
gggatgccaacccaaaaccaaccccctaacacatcccaaagagtaggtcgtattcatggaagga

SEQ ID NO: 128 - RNA, 79 nt, synthetic construct
  source 1..79: mol_type = other RNA
gggatgagtcaccccaacctgccctaccacggactcatccctccttccatgaacgccaaaccgtggcgacgacctactc

SEQ ID NO: 129 - DNA, 25 bp, synthetic construct
  source 1..25: mol_type = other DNA
tgtgggggtggacgggccgggtaga

SEQ ID NO: 130 - DNA, 25 bp, synthetic construct
  source 1..25: mol_type = other DNA
tgtgggggtggactgggtgggtacc

SEQ ID NO: 131 - DNA, 25 bp, synthetic construct
  source 1..25: mol_type = other DNA
tgtggggatggatgggccgggctgc

SEQ ID NO: 132 - DNA, 25 bp, synthetic construct
  source 1..25: mol_type = other DNA
tgtgggggtggacgggccgagtacg

SEQ ID NO: 133 - DNA, 25 bp, synthetic construct
  source 1..25: mol_type = other DNA
tgtgggggtggttgggcggggctgc

SEQ ID NO: 134 - DNA, 25 bp, synthetic construct
  source 1..25: mol_type = other DNA
tgtgggggaggacgggcggggatgc

SEQ ID NO: 135 - DNA, 25 bp, synthetic construct
  source 1..25: mol_type = other DNA
tgtggggttggacggaccgggtagg

SEQ ID NO: 136 - DNA, 25 bp, synthetic construct
  source 1..25: mol_type = other DNA
tgtggggttggacgggccgtgtaga

SEQ ID NO: 137 - DNA, 25 bp, synthetic construct
  source 1..25: mol_type = other DNA
tgtgggggtgcgcgggccgggcagg

SEQ ID NO: 138 - DNA, 25 bp, synthetic construct
  source 1..25: mol_type = other DNA
tgtgcggggggtgggggtggttccc

SEQ ID NO: 139 - DNA, 25 bp, synthetic construct
  source 1..25: mol_type = other DNA
tgtggggggggtgggggtggaccgc

SEQ ID NO: 140 - DNA, 25 bp, synthetic construct
  source 1..25: mol_type = other DNA
tgtgcgggtggccggggtggcctgc

SEQ ID NO: 141 - DNA, 25 bp, synthetic construct
  source 1..25: mol_type = other DNA
tgtggggctgggaggtcgggcctgc

SEQ ID NO: 142 - DNA, 18 bp, synthetic construct
  source 1..18: mol_type = other DNA
tttttgccacaccgtggc

SEQ ID NO: 143 - DNA, 14 bp, synthetic construct
  source 1..14: mol_type = other DNA
cggtgtggcacccg

SEQ ID NO: 144 - DNA, 37 bp, synthetic construct
  source 1..37: mol_type = other DNA
ccctactgttagggggttggttttgggttgggtaggg

SEQ ID NO: 145 - DNA, 25 bp, synthetic construct
  source 1..25: mol_type = other DNA
aagttaattagtatgtggatgtaga

SEQ ID NO: 146 - DNA, 17 bp, synthetic construct
  source 1..17: mol_type = other DNA
ctacatccacatcacta

(19) United States
(12) Patent Application Publication
(10) Pub. No.: US 2026/0328756 A1
(43) Pub. Date: Oct. 22, 2026

(54) APTAMER REGULATED TRANSCRIPTION FOR IN-VITRO SENSING AND TRANSDUCTION

(71) Applicants: The Johns Hopkins University, Baltimore, MD (US); Government of the United States of America, as Represented by the Secretary of Commerce, National In, Gaithersburg, MD (US)

(72) Inventors: Rebecca B. Schulman, Kingsville, MD (US); Heonjoon Lee, Baltimore, MD (US); Samuel W. Schaffter, Gaithersburg, MD (US)

(21) Appl. No.: 19/133,959

(22) PCT Filed: Nov. 29, 2023
(86) PCT No.: PCT/US2023/081639

§ 371 Date: May 29, 2025

Related U.S. Application Data
(60) Provisional application No. 63/385,230, filed on Nov. 29, 2022.
(60) Provisional application No. 63/516,727, filed on Jul. 31, 2023.

(51) Int. Cl. G01N 33/53; C12N 15/115; C12Q 1/6897

(52) U.S. Cl. CPC G01N 33/5308; C12N 15/115; C12Q 1/6897; C12N 2310/16

(57) ABSTRACT

Described are nucleic acid transcription templates, systems and methods for detection and measurement of molecules and biomolecules (e.g., biomarkers). Particularly, systems and methods utilize a transcription template with an aptamer domain configured to bind a molecule of interest to regulate production of a transcribed output RNA.

### STATEMENT REGARDING FEDERALLY SPONSORED RESEARCH OR DEVELOPMENT

This invention was made with government support under Grant No. CA251027, awarded by the National Institutes of Health, and Grant No. W911NF-20-1-0057, awarded by the United States Army Research Office. The government has certain rights in the invention.

### TECHNICAL FIELD

The present disclosure relates to nucleic acid transcription templates, systems and methods for detection and measurement molecules and biomolecules (e.g., biomarkers). Particularly, systems and methods utilize a transcription template with an aptamer domain configured to bind a molecule of interest to regulate production of a transcribed output RNA.

### CROSS-REFERENCE TO RELATED APPLICATIONS

This application claims the benefit of U.S. Provisional Application Nos. 63/385,230, filed Nov. 29, 2022, and 63/516,727, filed Jul. 31, 2023, the contents of which are herein incorporated by reference in their entirety.

### SEQUENCE LISTING STATEMENT

The content of the electronic sequence listing titled JHU_41376_601_SequenceListing.xml (Size: 133,250 bytes; and Date of Creation: Nov. 28, 2023) is herein incorporated by reference in its entirety.

### BACKGROUND OF THE INVENTION

Various human diseases are characterized by changes in the expression patterns of potential biomarkers, such as proteins, nucleic acids, and metabolites. While numerous biosensing strategies have been developed, the inability to accurately translate discrete levels of biomolecular signals into desirable information remains a major challenge in the field. New systems and methods are needed to expand the available tools for molecular detection and measurement.

### SUMMARY OF THE INVENTION

The disclosure provides nucleic acid transcription templates. In some embodiments, the nucleic acid transcription templates comprise an aptamer region configured to bind a molecule downstream of a promoter region and upstream of an output region which encodes an RNA output. In some embodiments, the nucleic acid transcription template is DNA. In some embodiments, the promoter region and/or the output region is double stranded.

In some embodiments, the aptamer region is flanked on each side by stem sequences. In some embodiments, the stem sequences are each 2-20 bases in length. In some embodiments, the stem sequences are each 5-7 bases in length. In select embodiments, the stem sequences are each 6 bases in length. In some embodiments, the aptamer region is single stranded.

Also provided herein are systems for the detection and/or measurement of at least one molecule. In some embodiments, the systems comprise at least one first nucleic acid transcription template comprising an aptamer region configured to bind to the at least one molecule downstream of a promoter region and upstream of an output region which encodes at least one first RNA output; and an RNA polymerase. In some embodiments, production of the at least one first RNA output is repressed in the presence of the at least one molecule.

In some embodiments, the nucleic acid transcription template is DNA. In some embodiments, the promoter region and/or the output region is double stranded.

In some embodiments, the aptamer region is flanked on each side by stem sequences. In some embodiments, the stem sequences are each 2-20 bases in length. In some embodiments, the stem sequences are each 5-7 bases in length. In select embodiments, the stem sequences are each 6 bases in length. In some embodiments, the aptamer region is single stranded.

In some embodiments, the systems further comprise one or more modules and/or components which utilize the at least one first RNA output as an input for signal processing, in vitro transcription networks and circuits, chemical reactions, or a combination thereof.

In some embodiments, the systems further comprise a nucleic acid reporter configured for detecting one or more first RNA output. In some embodiments, the nucleic acid reporter is double stranded comprising a first strand complementary to one or more of the at least one RNA output and a second strand with a detection moiety. In some embodiments, the detection moiety comprises a radioisotope, mass isotope, dye, fluorophore, or hapten.

In some embodiments, the systems further comprise an RNA output inversion module. In some embodiments, the RNA output inversion module comprises: a second nucleic acid transcription template comprising at least a partially singled stranded promoter site and an output region encoding a second RNA output; and a first nucleic acid activator strand which is complementary to the partially single stranded promoter site and the first RNA output. In some embodiments, the second nucleic acid transcription template is configured to transcribe the second RNA output when the first nucleic acid activator strand is bound to the partially single stranded promoter. In some embodiments, production of the second RNA output is increased in the presence of the molecule.

In some embodiments, the systems further comprise an RNA output amplification module.

In some embodiments, the RNA output amplification module comprises: a third nucleic acid transcription template comprising a partially single stranded promoter site and a sequence encoding a third RNA output; a second nucleic acid activator strand which is complementary to the partially single stranded promoter site on the third nucleic acid transcription template; and a nucleic acid blocker strand complementary to the second RNA output and the single stranded promoter site on the third nucleic acid transcription template. In some embodiments, the nucleic acid blocker strand is configured to prevent transcription from the third nucleic acid transcription template. In some embodiments, the first nucleic acid activator strand, the second nucleic acid activator strand, and/or the nucleic acid blocker strand is DNA.

In some embodiments, the systems further comprise a self-calibration module comprising a reference nucleic acid transcription template lacking a single stranded aptamer domain and a sequence encoding a reference RNA output. In some embodiments, the reference nucleic acid transcription template and the first nucleic acid transcription template are configured to transcribe at the same rate. In some embodiments, the at least one first RNA output and the reference RNA output comprise complementary sequences. In some embodiments, the self-calibration module comprises a reference reporter nucleic acid. In some embodiments, the reference reporter nucleic acid is complementary to the reference RNA output.

In some embodiments, the systems are embedded in a hydrogel. In some embodiments, at least one of the nucleic acid transcription template and the nucleic acid reporter are linked to a matrix molecule of the hydrogel. In some embodiments, at least one of: the second nucleic acid transcription template, first nucleic acid activator strand, third nucleic acid transcription template, second nucleic acid activator strand, nucleic acid blocker strand, reference nucleic acid transcription template are linked to a matrix molecule of the hydrogel.

In some embodiments, the systems further comprise a ribonuclease.

In some embodiments, the systems further comprise a sample. In some embodiments, the sample is a biological sample. In some embodiments, the molecule is a biomolecule. In some embodiments, the molecule is a biomarker. In some embodiments, the molecule is a protein.

In some embodiments, the systems further comprise an RNA detection component.

Further provided are methods for detection or measuring the level of at least one molecule. In some embodiments, the methods comprise incubating a sample comprising the at least one molecule with a system disclosed herein and measuring the at least one first RNA output or a signal corresponding to the at least one first RNA output. In some embodiments, measuring a signal corresponding to the at least one first RNA output comprises measuring the second RNA output, the third RNA output and/or the reference RNA output.

In some embodiments, the incubating is done under conditions to promote transcription. In some embodiments, the conditions to promote transcription comprises incubating the system and sample in a reaction mixture comprising any or all of: nucleoside triphosphates, buffers, cofactors, detergents, reducing agents, small molecule additives, cell culture media, and enzymatic additives.

In some embodiments, the sample is a biological sample. In some embodiments, the molecule is a biomolecule. In some embodiments, the molecule is a biomarker. In some embodiments, the molecule is a protein.

In some embodiments, the system comprises two or more first transcription templates directed to two or more different molecules. In some embodiments, the two or more different molecules are biomolecules. In some embodiments, the two or more different biomolecules are biomarkers.

In some embodiments, the methods further comprise determining the presence or risk of a disease or disorder based on the RNA output.

Other aspects of the invention will become apparent by consideration of the detailed description and accompanying drawings.

### BRIEF DESCRIPTION OF THE DRAWINGS

FIG. 1 is a schematic of an exemplary Aptamer Regulated Transcription for In-vitro Sensing and Transduction (ARTIST) workflow. (i) ARTIST takes in proteins as inputs, which interact with dART (DNA for aptamer regulated transcription) templates configured to produce an RNA output which can be detected in various ways, including using a reporter nucleic acid as shown. Depending on the transient protein concentration in the system, ARTIST can convert the output to dose-dependent digital signals of input protein based on repressed transcriptional behavior. (ii) ARTIST uses a sensing mechanism in which when there is no protein to bind to the aptamer, dART actively transcribes to produce the RNA output. However, when a protein binds to the aptamer domain, the dART exhibits repressed RNA output. (iii) ARTIST outputs can be transduced in diverse ways such as signal inversion, amplification, or sensor self-calibration.

FIG. 2 is a schematic of an exemplary sensing mechanism for use with ARTIST. When there is no protein in the system to bind to the aptamer domain, T7 RNA polymerase (T7 RNAP) is able to use the dART template to actively transcribe RNA, resulting in an RNA output which can be measured. When the corresponding protein, however, is present, it can bind to the aptamer, which then serves as a blockade for transcription. This would repress RNA output production even in the presence of T7 RNAP.

FIG. 3 shows schematic illustrations of an exemplary dART template and the resulting RNA output with the different domains. NUPACK analysis confirms a single-stranded aptamer domain downstream of the promoter and initiation domains to repress transcription. The analysis also shows that the aptamer′ domain is sequestered from the output s domain by a hairpin stem (6 basepair). This prevents unwanted secondary structure formation between the aptamer′ and s domain sequences.

FIGS. 4A and 4B show the relationship between length of initiation (i) domain and dART template transcription. FIG. 4A is a schematic illustration of a dART with different lengths for the i/i′ domains. FIG. 4B is time-dependent transcription profiles of the IFN-γ dARTs using varying lengths of i/i′ domains with 0 or 100 nM of IFN-γ.

FIGS. 5A and 5B show protein sensitivity and selectivity of ARTIST. FIG. 5A is dose-response transcription curves of various dARTs configured for various proteins (e.g., IFN-γ, IL-6, thrombin, TNF-α) with varying levels of their corresponding proteins (0-1000 nM). Here, 10 nM of dART and 100 nM DNA reporter were used. FIG. 5B is a selectivity heat map of dARTs to different proteins concentrated at 100 nM. Herein, 10 nM of dART was used per assay, and transcription rate was measured for the first hour of reaction.

FIGS. 6A and 6B show regulation of RNA output with steady-state production and degradation rates. FIG. 6A is a schematic illustration of RNA production regulated by T7 RNAP and RNase H. For an active dART with no protein bound to the aptamer, T7 RNAP is able to transcribe RNA which turns on the reporter. RNase H, however, is able to degrade the RNA hybridized to the quencher strand, which can reattach to the single-stranded fluorophore DNA. These counteractive interactions of T7 RNAP and RNase H induce steady state production of RNA. When protein is bound to the dART, RNA production is repressed even in the presence of T7 RNAP. FIG. 6B is dose-response transcription curves of various dARTs configured for various proteins (e.g., IFN-γ, IL-6, thrombin, TNF-α) with varying levels of their corresponding proteins (0-1000 nM). Here, 10 nM of dART and 100 nM DNA reporter were used.

FIG. 7 shows a schematic of an exemplary inverter circuit for use with ARTIST. G1S1 is intrinsically transcriptionally inactive (OFF state). It can be turned on when the DNA activator strand (dA1) binds and turns it to ON state. However, dART-R1 can inhibit activation of G1S1 as it transcribes rR1 that sequesters the dA1 strand. When protein is bound to dART-R1, however, dA1 is then able to bind to G1S1. G1S1 can transcribe rS1, and the output signal is tracked using a DNA reporter.

FIG. 8 shows measurement of RNA output using an inverter circuit with varying concentrations of IFN-γ. Experimental (left) and simulated (right) time-dependent profiles of output signal production of the inverter circuit with varying concentrations (0, 25, 250, 2500 nM) of IFN-γ. Herein, 25 nM of dART-R1, 25 nM of G1S1, 250 nM of DNA reporter were used.

FIG. 9 shows amplification of RNA output using an inverter circuit with low (25 nM) concentrations of IFN-γ. Experimental (left) and simulated (right) amplification of output signal production for the inverter circuit with 25 nM of IFN-γ. Herein, 25 nM of dART-R1, 25-100 nM of G1S1, 250 nM of DNA reporter were used.

FIG. 10 shows amplification of RNA output using an inverter circuit having increased G1S1 concentrations. Measuring leak of the amplified inverter circuit with 25-100 nM of G1S1 and 0 or 25 nM of IFN-γ. Leak is defined as the output signal of the inverter circuit with 0 nM of IFN-γ.

FIG. 11 shows the design and use of an exemplary inverter-amplifier circuit with ARTIST. Left: Schematic illustration of the integrated inverter and amplifier circuit. Intrinsically G2C1 is OFF and G1S1 is blocked by a DNA blocker (dB1). dART-R2 can inhibit activation of G2C1, as it transcribes rR2. When protein is bound to dART-R2, however, dA2 is able to bind to G2C1, which turns on the node and allows transcription of rC1. rC1 is then able to hybridize with dB1 and displace off of G1S1. dA1 is then able to bind to the unblocked G1S1, which can transcribe rS1. The output signal of rS1 is then tracked using a DNA reporter. Right: Normalized output signals of the inverter-amplifier circuit with 0 or 25 nM of IFN-γ. Herein, 25 nM of dART-R1, 50 nM of G2C1, 50 nM G1S1, and 250 nM of DNA reporter were used. The concentration of was varied dB1 from 50-250 nM, as indicated.

FIG. 12 shows the design and use of a bistable switch with ARTIST. Left: When dART-R1 is not bound to the protein, rR1 transcribed is able to sequester the inducer RNA (rR1) for state change. Hence, the bistable switch remains turned off. Right: When the protein is bound to the dART, rR1 is able to inhibit the output of G4R1, thus inducing state change.

FIG. 13 shows a design of an exemplary comparator circuit for use with ARTIST. A reference template and dART transcribe at the same rate, and accordingly there will be no reporter turned on as the double-stranded output do not allow signal generation from the reporter nucleic acid. When the protein is present and able to bind to dART, the reference template will be transcribed as a higher rate and be able to turn on the DNA reporter.

FIGS. 14A and 14B show the dose-response of a comparator circuit for various T7 RNAP concentrations. FIG. 14A is time-dependent simulated and experimental transcription profiles of the comparator with different concentrations of IFN-γ using 2, 4, and 8 U/uL concentrations of T7 RNAP. FIG. 14B is IFN-γ dose-response curves of the comparator circuit with different concentrations of T7 RNAP.

FIG. 15 is a schematic showing the use of a comparator circuit with an amplifier. Similar to the scheme shown in FIG. 11, the coactivator can remove blockers from G1:S1:dB1. However, rC1 is transcribed by the reference template, and the dART without binding to the protein can transcribe the repressor RNA (rR1) which can sequester rC1 and prevent its interaction with G1S1:dB1. If the target protein is present, dART is unable to transcribe rR1, and rC1 can unblock G1:S1:dB1.

FIG. 16 shows a schematic of multiplexed diagnostics for use with ARTIST. Multiple proteins are taken as inputs to bind to their corresponding dART templates. Through summation of ARTIST reactions, depending on how much signal the reference template produces the classifier determines whether or not the combination of protein levels indicate a disease state.

FIGS. 17A-17B are schematics of the ARTIST platform. FIG. 17A shows that in the absence of the aptamer's protein ligand, dARTs are transcribed to produce an RNA output (left), but protein binding represses transcription (middle). The input and output domains are decoupled (right), which enables modular design of dARTs by swapping out the aptamer domain or customizing the output sequences to encode different RNA outputs. DNA and RNA are represented with solid and dashed lines, respectively. FIG. 17B shows that dARTs serve as the protein sensing layer (left) whose outputs can be coupled with downstream circuits to demonstrate versatile functionalities (middle). The RNA output of molecular circuits can react with a DNA reporter complex (right), which produces measurable fluorescence for detection.

FIGS. 18A-18I show the design of aptamer regulated transcription templates (dARTs). (FIG. 18A) dART templates consist of a double-stranded promoter region (pink boxed region), a single-stranded aptamer domain on the template strand read by T7 RNAP (Apt), and a double-stranded output domain (O1). The single-stranded aptamer domain permits aptamer-ligand binding. (FIG. 18B) Secondary structure of the RNA transcript predicted by NUPACK (SEQ ID NO: 85). (FIG. 18C) The aptamer sequences of IFN-O1-dART, which binds IFN-γ and Dummy-O1-dART, whose aptamer domain does not have a tertiary structure or specific protein affinity. (FIG. 18D) dART transcripts react with an O1 DNA Reporter via toehold-mediated strand displacement. (FIG. 18E) Reacted reporter kinetics from IFN-O1-dART and Dummy-O1-dART transcription with and without IFN-γ and potassium. (FIG. 18F) Simulated and experimental dose-response curves for 10 nM IFN-O1-dART with 0 nM to 1000 nM IFN-γ for K_{d,apparent}=8 nM. Three independent replicates (black) and their mean (blue) are plotted. The dashed line is a simulation for K_{d,apparent} matching experiments. The shaded region shows predictions for K_{d,apparent} values between 1 nM to 20 nM (see Methods). (FIG. 18G) Experimentally measured (bold) and simulated (dashed) reacted reporter kinetics for 10 nM of IFN-O1-dART and 0 nM to 1000 nM of IFN-γ. (FIG. 18H) Simulated and experimental dose-response curves and (FIG. 18I) Experimental (bold) and simulated (dashed) reacted reporter kinetics for 1 nM IFN-O1-dART and 0 nM to 100 nM of IFN-γ using the K_{d,apparent} determined in FIG. 18F. For FIGS. 18F and H, the reacted reporter concentration for each plot was measured at 120 min.

FIGS. 19A-19E show dART sensitivity, selectivity, and modularity. (FIG. 19A) A series of dARTs with different inputs, achieved by swapping aptamer domain sequences. (FIG. 19B) Reacted reporter kinetics by IFN-O1-dART, Thr-O1-dART, IL6-O1-dART, and TNF-O1-dART in the presence of 0 nM to 1000 nM IFN-γ, thrombin, IL-6, and TNF-α, respectively. Shaded regions represent minimum/maximum values of reacted reporter concentration of three independent replicates. (FIG. 19C) Heat map showing reacted reporter concentrations for 10 nM Thr-O1-dART, IFN-O1-dART, IL6-O1-dART, TNF-O1-dART, and Dummy-O1-dART (a control) each subjected to 100 nM of BSA, thrombin, TNF-α, IL-6, or IFN-γ. (FIG. 19D) A series of dARTs with different outputs, achieved by swapping template output sequences. (FIG. 19E) Reacted reporter kinetics for IFN-O1-dART, IFN-O2-dART, and IFN-O3-dART for 0 nM to 1000 nM of IFN-γ.

FIGS. 20A-20C show Analog biosensors with steady-state outputs. (FIG. 20A) Schematic of coupled RNA transcription of dARTs by T7 RNAP and RNA degradation by RNase H. (FIG. 20B) The concentration of reacted reporter over time for an experiment with 10 nM IFN-O1-dART, 0 nM to 1000 nM of IFN-γ, 2 U μL⁻¹ of T7 RNAP, and 2×10⁻³ U μL⁻¹ of RNase H. Shaded regions enclose the minimum and maximum values for independent trials (N=2; see Methods). (FIG. 20C) Steady-state analog outputs of IFN-O1-dART, Thr-O1-dART, IL6-O1-dART, and TNF-O1-dART with 5 nM to 1000 nM of their corresponding proteins. Error bars represent minimum and maximum values of two independent replicates.

FIGS. 21A-21J show Comparator circuits for digital biosensing. (FIG. 21A) The comparator circuit. When [IFN-γ] is high, IFN-O1′-dART's transcription rate is low. Ref-O1-RNA is thus produced in excess, and it reacts with the O1 DNA Reporter. When [IFN-γ] is low, IFN-O1′-RNA's transcription rate is higher than Ref-O1-RNA's, and Ref-O1-RNA is sequestered by IFN-O1′-RNA before it can react with the reporter. (FIG. 21B). Left: when [IFN-γ] is high, reacted reporter rises concentration should increase rapidly until all reporter is reacted, When [IFN-γ] is low it should rise slowly or not at all. Right: the concentration of reacted reporter should therefore either be fully ON or very low (OFF) at the end of the reaction. (FIG. 21C) Simulated kinetics of IFN-O1-C-50. (FIG. 21D) Experimentally measured kinetics of IFN-O1-C-50. (FIG. 21E) Dose-response curve of IFN-O1-C-50 after 240 min of reaction. Dashed lines represent simulations, whereas the points represent experimental values. (FIG. 21F) The dose-response curves of IFN-O1-C-30, IFN-O1-C-50, and IFN-O1-C-100. (FIG. 21G) The comparator is designed to K+-insensitive. IFN-O1′-dART and Ref-O1-dART both form G-quadruplexes, and their transcription rates should change similarly with [K+] concentrations so the output of the comparator should be relatively insensitive to [K+]. (FIG. 21H) Dose-response curves of IFN-O1-C-50 in FIG. 21G; responses were measured after 240 min. The line plots are added to aid visualization of the trends in the reacted reporter concentrations for each [K+] concentration. (FIG. 21I) A [K+]-sensitive comparator. At high [K+], the aptamer domain on the IFN-O1′-dART forms a G-quadruplex, which reduces its transcription rate. If the IFN-O1′-dART's output is compared to an RNA produced at a rate insensitive to [K+](from Dummy-O1-dART) output should change with [K+]. (FIG. 21J) Dose-response curves of IFN-O1-C-50 with Dummy-O1-dART for different [K+]; responses were measured after 240 min. The line plots are added to aid visualization of the trends for each [K+] concentration.

FIGS. 22A-22E show sensitive protein detection with amplified comparators. (FIG. 22A) Circuit diagram for an amplified IFN-C1′-dART/Ref-C1-dART comparator. The Ref-C1-RNA output, which is high when IFN-γ exceeds a threshold concentration, activates transcription of a genelet that transcribes O4, which is detected by a reporter. (FIG. 22B) The reactions through which Ref-C1-RNA coactivates G1O4 to produce the O4 output. (FIG. 22C) Reacted reporter kinetics of IFN-O4-C-50-G1O4, which consists of 25 nM Ref-C1-dART, 50 nM IFN-C1′-dART, 100 nM G1O4:B1, 200 nM A1, and 2000 nM O4 DNA Reporter, for 0 nM or 50 nM IFN-γ. (FIG. 22D) Reacted reporter kinetics of the IFN-O4-C-1-G1O4, which consists of 0.5 nM of Ref-C1-dART, 1 nM IFN-C1′-dART, 20 nM of G1O4:B1, and 100 nM of A1, and 250 nM of O4 DNA Reporter for IFN-γ inputs from 0 nM to 4 nM. 4 U μL⁻¹ T7 RNAP and 4×10⁻³ U μL⁻¹ RNase H were used in IFN-O4-C-1-G1O4. (FIG. 22E) Comparison of the dose-response curves of IFN-O4-C-1-G1O4, IFN-O1-C-30, IFN-O1-C-50, and IFN-O1-C-100. The line plots are added to aid visualization of the trends for each dose-response curve.

FIGS. 23A-23B show NUPACK predicts that insulator domains prevent the formation of unintended secondary structure within dART RNA outputs. FIG. 23A is a transcript from IFN-O1-dART containing a i′ domain is predicted to form a hairpin structure, leaving the output (O1) domain free (SEQ ID NO: 88). FIG. 23B shows undesired secondary structure forms in a transcript from a dART without the i′ domain: bases from the O1 domain hybridize with i (SEQ ID NO: 89). This secondary structure could prevent or alter the rate of the output's participation in downstream reactions such as strand displacement.

FIGS. 24A-24B show graphs of the normalization of reacted reporter measurements. FIG. 24A is representative examples of how time-dependent measurements of reacted reporter concentration were obtained from fluorescence measurements. 10 nM of IFN-O1-dART, 100 nM of O1 DNA Reporter, 0 nM, or 100 nM of IFN-γ are initially combined. Fluorescence readings were first taken for 10 min. 2 U μL⁻¹ of T7 RNAP was then added. After measuring fluorescence for another 240 min to 360 min, 2.5 μM of O1-DNA, which is fully complementary to o1′_q, was mixed into each sample to obtain an internal maximum DNA reporter fluorescence value. At least ten minutes after adding in O1-DNA, maximum fluorescence was measured. Since the reacted fluorescent signal is proportional to the extent of the reaction, the concentration of reacted reporter could be obtained from the fluorescence using Eqn 1. FIG. 24B shows the concentrations of reacted reporter over time for reactions containing 10 nM of IFN-O1-dART and 100 nM of O1 DNA Reporter with either 0 nM (top) or 100 nM (bottom) of IFN-γ.

FIG. 25 is a schematic of sequences and domains for the dART variants and their RNA transcripts with i₂, i₄, and i₆ between the promoter and aptamer domains. Sequences highlighted in teal can hybridize to the h domain to form a hairpin. The h domain is in teal font. The i_6 dART variant was used to design all other dARTs.

FIG. 26 is a schematic of sequences and domains for the dART variants and their RNA transcripts with leader domains of varying length (+4, +8, +12, and +16) and i₆ between the promoter and aptamer domains. Leader domains are in orange font.

FIG. 27 is graphs of reacted reporter kinetics induced by dARTs with varying lengths between the promoter and aptamer (2 to 22) bases and 0 nM or 100 nM IFN-γ. For all measurements, 10 nM dART and 100 nM reporter were used. Shaded regions denote minimum/maximum values of reacted reporter concentrations for three independent replicates.

FIGS. 28A and 28B show schematics of reactions in the kinetic model. FIG. 28A shows an unbound dART can be recognized by T7 RNAP which transcribes an output with a rate constant of kₜₓₙ. However, a dART bound to its protein ligand cannot undergo transcription to produce RNA. FIG. 28B shows the RNA transcript produced from an unbound dART can bind to the 6-base toehold on a DNA Reporter complex and undergo a toehold-mediated strand displacement reaction with a rate constant of k_{sd}. The measured output of this reaction is [Reporter]_{reacted}, the concentration of a fluorescent DNA strand.

FIG. 29 is a graph of fitting the transcription rate of 10 nM IFN-O1-dART and 0 nM of IFN-γ. A kₜₓₙ value of 0.0015 s⁻¹ was fit to the average transcription plot that was measured experimentally. Shaded regions represent minimum/maximum values of reacted reporter concentration for three independent replicates.

FIGS. 30A-30C are simulated and experimental dose-response curves for 10 nM of IFN-O1-dART detecting 0 nM to 100 nM of IFN-γ with K_{d,apparent} of 1.6 nM (FIG. 30A), 8 nM (FIG. 30B), and 40 nM (FIG. 30C). Based on our results, 8 nM is the most suitable value of K_{d,apparent} between IFN-O1-dART and IFN-γ to fit the observed dose-response behavior. For 24A-24C, error bars show the standard deviations of three independent replicates. The simulated and experimental dose-response curves for K_{d,apparent} of 8 nM are also shown in FIG. 18E.

FIG. 31 is sequences of the aptamer domains of IFN-O1-dART, Thr-O1-dART, IL6-O1-dART, and TNF-O1-dART. Full sequences of the dART strands are provided below.

FIG. 32 is RNA secondary structures of the encoded RNA transcripts of IFN-O1-dART, Thr-O1-dART, IL6-O1-dART, and TNF-O1-dART predicted by NUPACK.

FIG. 33 is graphs of reacted reporter kinetics for 10 nM of IFN-O1-dART, Thr-O1-dART, IL6-O1-dART, TNF-O1-dART, and a Dummy-O1-dART (a control) subjected to 100 nM of BSA, IFN-γ, thrombin, IL-6, or TNF-α. The heat map in FIG. 19C shows the concentration of reacted reporter after 120 min for IFN-O1-dART, Thr-O1-dART, IL6-O1-dART, TNF-O1-dART, and Dummy-O1-dART with each protein.

FIG. 34 is output sequences of IFN-O1-dART, IFN-O2-dART, and IFN-O3-dART.

FIG. 35 is RNA secondary structures of the IFN-RNA encoded by IFN-O1-dART, IFN-O2-dART, and IFN-O3-dART predicted by NUPACK.

FIG. 36 is sequences of the aptamer domains of KMy-O1-dART and VEGF-O1-dART. Full sequences of the dART strands are provided below.

FIG. 37 is predicted RNA secondary structures of KMy-O1-RNA and VEGF-O1-RNA. The G-U wobble in the VEGF-O1-RNA is highlighted in cyan. Secondary structure predictions of the transcripts were done using NUPACK.

FIGS. 38A and 38B show the reacted reporter kinetics for the KMy-O1-dART (FIG. 38A) and the VEGF-O1-dART (FIG. 38B). 10 nM of KMy-O1-dART or VEGF-O1-dART was mixed with 100 nM of O1 DNA Reporter with or without KCl and with or without ligand as indicated in the plots.

FIG. 39 shows analog biosensors in which the steady-state concentration of reacted reporter indicates protein concentration. Reacted reporter concentration over time of analog biosensors for IFN-γ, thrombin, TNF-α, and IL-6. 10 nM IFN-O1-dART, Thr-O1-dART, IL6-O1-dART, and TNF-O1-dART were mixed with 100 nM of reporter, and 0 nM to 1000 nM of their corresponding input proteins. 2 U μL⁻¹ of T7 RNAP and 2×10⁻³ U μL⁻¹ of RNase H were used in each assay. Shaded regions enclose the minimum and maximum values of reacted reporter concentration for two replicates. The IFN-O1-dART data is also shown in FIG. 20B. Endpoint measurements of reacted reporter concentrations at 240 min are shown in FIG. 20C.

FIGS. 40A and 40B show the reaction between IFN-O1′-RNA and Ref-O1′RNA. FIG. 40A shows that when IFN-γ is not bound to IFN-O1′-dART, IFN-O1′-RNA is produced and can hybridize with Ref-O1-RNA. FIG. 40B shows the secondary structure of the IFN-O1′-RNA/Ref-O1-RNA complex predicted by NUPACK. The hybridization of the O1 and O1′ output domains prevents the reaction of Ref-C1-RNA with the O1 DNA Reporter.

FIGS. 41A-41C show the reactions in the model of the comparator. FIG. 41A shows that IFN-O1′-RNA and Ref-O1-RNA are transcribed from unbound IFN-O1′-dART and Ref-O1-dART, respectively. Transcription reactions are modeled as unimolecular reactions with the same effective reaction rate kₜₓₙ. FIG. 41B shows that Ref-O1-RNA and IFN-O1′-RNA hybridize via a thresholding rate constant kₜₕ. FIG. 41C shows that Ref-O1-RNA reacts with the O1 reporter in a 6 base toehold-mediated strand displacement reaction with rate constant k_{sd}.

FIGS. 42A-42C show the identification of which concentrations of unbound IFN-O1′-dART produce on/off comparator outputs. FIG. 42A shows simulated kinetics of reacted reporter from the comparator with 0 nM to 100 nM of unbound IFN-O1′-dART and 25 nM of Ref-O1-dART (kₜₓₙ=0.004 s⁻¹). FIG. 42B shows the experimental kinetics of reacted reporter for the comparator with 0 nM to 100 nM of IFN-O1′-dART and 25 nM of Ref-O1-dART. FIG. 42C shows the simulated and experimentally measured reacted reporter concentrations after 240 min plotted vs. unbound IFN-O1′-dART concentration.

FIGS. 43A and 43B show simulated (FIG. 43A) and experimental (FIG. 43B) reacted reporter kinetics of the comparator with 0 nM to 100 nM of IFN-γ and three different T7 RNAP concentrations. For the simulations, kₜₓₙ values of 0.002 s⁻¹, 0.004 s⁻¹, and 0.008 s⁻¹ were used to estimate the rates of transcription at T7 RNAP concentrations of 2 U μL⁻¹, 4 U μL⁻¹, and 8 U μL⁻¹, respectively. The amount of T7 RNAP used in each experiment is shown as the title. The 4 U μL⁻¹ data is also shown in FIG. 21D.

FIG. 44 shows simulated (dashed) and experimental (bold) dose-response curves for the comparator for three different T7 RNAP concentrations as shown in the legend. Reacted reporter concentrations were measured after 240 min of reaction.

FIG. 45 shows reacted reporter concentrations of the comparator for 0 nM to 100 nM of IFN-γ with varying concentrations of potassium ions (20 mM to 100 mM). The comparator demonstrates digital responses to low (0 nM to 20 nM) and high (50 nM to 100 nM) concentrations of IFN-γ. The dose-response curves in FIG. 21H were obtained from the endpoint reacted reporter concentrations shown in these plots. As [K+] increased, the baseline signal, defined as the concentration of reacted reporter without any input of IFN-γ, decreased. This decrease could be caused by higher [K+] facilitating stable G-quadruplex formation, which would reduce the rate of transcription of both dARTs, analogous to reducing the global transcription rate.

FIG. 46 shows reacted reporter concentrations over time of the dummy comparator, which compares the outputs of 25 nM Dummy-O1-dART and 50 nM IFN-O1′-dART for 0 nM to 100 nM of IFN-γ for three different concentrations of KCl, which are given above the corresponding plots. The dose-response curves in FIG. 21J were obtained from the endpoint reacted reporter concentrations shown in these plots.

FIGS. 47A and 47B show reactions between IFN-C1′-RNA and Ref-C1′-RNA. FIG. 47A shows that IFN-C1′-RNA is transcribed by unbound IFN-C1′-dART and hybridizes with Ref-C1-RNA. FIG. 47B shows that the secondary structure of the IFN-C1′-RNA:Ref-C1-RNA complex predicted by NUPACK. The hybridization of the c1 and c1′ output domains prevent Ref-C1-RNA from reacting with the G1O4 blocked genelet or B1 (FIG. 48) when it is bound to IFN-C1′-RNA.

FIG. 48 is a detailed schematic of the genelet reactions in the amplified comparator. When IFN-C1′-dART is bound to its protein ligand, Ref-C1-dART produces Ref-C1-RNA, which displaces B1 from G1O4:B1 to produce G1O4. A1 is then able to bind to G1O4; the resulting complex G1O4:A1 has a complete T7 promoter and can be transcribed to produce O4-RNA. The O4-RNA can react with the O4 DNA Reporter, which is the measured fluorescent output. B1 can bind to G1O4:A1, resulting G1O4:B1 and A1.

FIGS. 49A and 49B show the kinetics of the comparator with 0 nM or 50 nM IFN-γ and without (FIG. 49A) or with (FIG. 49B) RNase H (4×10⁻³ U μL⁻¹). The total DNA reporter concentration was 2000 nM.

FIGS. 50A-50C show graphs comparing sensitivity of the 0.02× amplified comparator to that of the 0.02× comparator. FIG. 50A shows the kinetics of the 0.02× amplified comparator for IFN-γ inputs from 0 nM to 4 nM. FIG. 50B shows the kinetics of the 0.02× comparator for IFN-γ inputs from 0 nM to 4 nM. FIG. 50C shows the comparison of dose-response curves of the 0.02× amplified comparator and the 0.02× comparator for IFN-γ inputs from 0 nM to 4 nM. For the dose-response curve, the reacted reporter concentrations were measured at 240 min. The kinetic data of the 0.02× amplified comparator is also shown in FIG. 22D.

FIGS. 51A and 51B show aptamer sequences which can bind to VEGF, adapted from Tanaka et al., (Anal. Chem. 85, 1132-1137 (2013)) and reacted reporter kinetics of 3R02-O1-dART, 3R03-O1-dART, 3R08-O1-dART, and 3R09-O1-dART with 0 or 100 mM of KCl, respectively. 3R02, 3R03, 3R08, and 3R09 correspond to the aptamer names that have been selected by Tanaka et al.

FIGS. 52A and 52B show the apparent binding affinity of 3R03-O1-dART and VEGF. FIG. 52A is a dose-response curve of 10 nM 3R03-O1-dART for 0 to 1000 nM of VEGF. Apparent K_{d} of 3R03-O1-dART was measured to be approximately 33 nM. Reacted reporter concentration was measured after 120 minutes of reaction. Error bars represent standard deviations of three independent replicates. FIG. 52B is a graph of the reacted reporter kinetics of 10 nM 3R03-O1-dART with 0, 5, 10, 100, and 1000 nM of VEGF.

FIGS. 53A-53C show a digital biosensor with the 3R03-based VEGF dART. FIG. 53A is a schematic illustration of the VEFG digital biosensor using 3R03-O1′-dART and Ref-O1-dART. FIG. 53B is a graph of simulated reacted reporter kinetics of a VEGF comparator that responds to 0-200 nM of VEGF. FIG. 53C is a simulated dose-response curve of the VEGF comparator. Reacted reporter concentrations are measured after 240 minutes of reaction.

FIG. 54 is a graph of the reacted reporter kinetics of 10 nMIFN-O1-dART with 0, 1, or 2 U μL⁻¹ RNase inhibitors in serum-supplemented culture media. All assays include 2 μM of actin to prevent degradation of the dART and the DNA reporter complex.

FIG. 55 is a schematic of an exemplary design and sequences for anchoring dARTs to hydrogels.

FIGS. 56A and 56B are schematics illustrating measuring ARTIST reactions in a hydrogel. dARTs are designed so that in the absence of IFN-γ, T7 RNA polymerase can transcribe IFN-O1-dART and produce an RNA transcript. The RNA transcript can react with a DNA reporter complex that is also anchored to the hydrogel. Once the RNA transcript reacts with the reporter the quencher is removed, and the hydrogel can produce a fluorescent measurement (FIG. 56A). When IFN-γ binds to IFN-O1-dART, T7 RNA polymerase cannot transcribe IFN-O1-dART (FIG. 56B).

FIG. 57 are graphs showing that hydrogel with ARTIST show decrease in reacted reporter kinetics with increasing protein concentrations. Hydrogels anchored with IFN-O1-dART show decrease in reacted reporter kinetics with increasing protein concentrations. Experimental reacted reporter kinetics appear to match well with those measured via simulation. The end-point reacted reporter measurements can be fit with the simulated dose-response curve of IFN-O1-dART for Kd,apparent of 8 nM.

FIG. 58 is time-lapse images of ARTIST hydrogel for IFN-γ with different IFN-γ concentrations. Hydrogel with IFN-O1-dART and 0 nM IFN-γ exhibits increasing fluorescence over time. Hydrogel with IFN-O1-dART and 100 nM IFN-γ exhibits minimal fluorescent signal increase over time.

### DETAILED DESCRIPTION OF THE INVENTION

Disclosed herein are systems and methods for cell-free molecular detection and measurement. The systems and methods function via aptamer regulated transcription for in-vitro sensing and transduction (ARTIST) of biomolecular signals. ARTIST comprises short transcription templates with an aptamer region located downstream of the promoter. The aptamer region is configured to bind specific ligands, which once bound results in transcription repression. As shown herein, the ARTIST templates are modular due to the ability to utilize different aptamers to target different proteins (e.g., IFN-γ, thrombin, TNF-α, IL-6) with high sensitivity and specificity. In addition, customizability of the RNA output sequences of these templates provides versatility for a variety of downstream detection methods and processes. For example, ARTIST was coupled with a variety of downstream circuits to enable signal inversion, signal amplification, and information processing. ARTIST is a facile, generalizable design for biosensors, allowing efficient monitoring of biomolecular signals and transduction of information in a multiplex fashion.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Complementarity” or “complementary to,” as used herein, refer to the ability of a nucleic acid to form hydrogen bond(s) with another nucleic acid sequence by either traditional Watson-Crick or other non-traditional types. Unless otherwise stated, “complementarity” or “complementary to” encompasses full or partial complementarity between the indicated sequences. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary).

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A second sequence that is complementary to a first sequence is referred to as the “complement” of the second sequence. The term “hybridizable” as applied to a polynucleotide refers to the ability of the polynucleotide to form a complex that is stabilized via hydrogen bonding between the bases of the nucleotide residues in a hybridization reaction.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine, and isoguanine. Nucleic acids may be obtained by chemical synthesis methods or by recombinant methods. Polynucleotides may be single stranded or double stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid sequence may be used for the same purpose as a given nucleic acid. Thus, a nucleic acid also encompasses substantially identical nucleic acids and complements thereof.

The term “promoter” refers to a cis-acting sequence that directs RNA polymerase and other trans-acting transcription factors to initiate RNA transcription from the template that includes the cis-acting sequence.

A “subject” or “patient” may be human or non-human and may include, for example, animal strains or species used as “model systems” for research purposes, such a mouse model as described herein. Likewise, subject may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### 2. TRANSCRIPTION TEMPLATES AND SYSTEMS

The present disclosure provides a nucleic acid transcription template comprising an aptamer region configured to bind to a molecule downstream of a promoter region and upstream of an output region which encodes an RNA output.

The disclosure further provides systems for the detection or measurement of the levels or concentrations of at least one molecule of interest. In some embodiments, the systems comprise at least one first nucleic acid transcription template comprising an aptamer region configured to bind to a molecule of interest downstream of a promoter region and upstream of an output region which encodes a first RNA output. In some embodiments, the systems comprise two or more first nucleic acid transcription templates, each configured to bind different molecules of interest. Thus, such systems allow simultaneous detection or measurement of two or more molecules.

As used herein, a transcription template, refers to a nucleic acid that serves as a substrate for transcribing an RNA, e.g., an RNA output. Transcription templates may include nucleic acid compositions having DNA, RNA, and/or modified nucleotides or bases, and may be linear or circular. In some embodiments, the transcription template is double stranded linear DNA. In some embodiments, the transcription template is a partially double stranded linear DNA.

The transcription template comprises an aptamer region. The aptamer region comprises a sequence that binds the molecule of interest. The aptamer region may comprise a previously identified sequence for molecule of interest or may be identified and selected for any molecule of interest using known methods, e.g., library-based methods, such as SELEX.

The molecule may be any analyte in need of detection such as (e.g., biomolecules (e.g., nucleic acids (e.g., DNA, RNA, methylated and other modified or non-naturally occurring nucleobases), polypeptides (e.g., peptides, proteins, glycoproteins), carbohydrates, lipids, amino acids, metabolites) and small molecules and other molecules. For example, embodiments provide for detection of an analyte that is a nucleic acid, a polypeptide, a carbohydrate, a polysaccharide, a fatty acid, a phospholipid, a glycolipid, a sphingolipid, a small molecule, a metabolite, a cofactor, etc.

In particular embodiments, the molecule of interest is a biomolecule. The biomolecule of interest may be a large macromolecule such as a protein, a nucleic acid, a carbohydrate, or lipid. In some embodiments, the biomolecule of interest is a protein. In some embodiments, the biomolecule of interest is a biomarker. A “biomarker” includes a biological compound, which is present in a biological sample and that may be isolated from or measured in a biological sample. Generally, a biomarker is associated with a given state of a subject, such as presence of a disease or disorder, risk of a disease or disorder, particular stage of disease or disorder.

The length of the aptamer region sequence is variable and dependent on the necessary sequence and structure elements which confer the aptamer its binding specificity. In some embodiments, the aptamer region comprises a sequence of about 10 to about 100 bases. In some embodiments, the aptamer region comprises a sequence of about 20 to about 50 bases. For example, the aptamer region may comprise a sequence of about 20, about 25, about 30, about 35, about 40, about 45, or about 50 bases.

In some embodiments, the aptamer region is single stranded. Commonly single stranded nucleic acids adopt secondary structures, for example, stems, loops, and the like. In some embodiments, the aptamer region adopts one or more secondary structures. The likely occurrence of secondary structure(s) within a nucleic acid sequence can be predicted through computational modeling known in the art, for example Nucleic Acid Package (NUPACK) (Zadeh, J N, et al., Journal of Computational Chemistry (2011), Vol. 32(1), 170-173).

In some embodiments, the aptamer region is flanked on each side by stem sequences. In some embodiments, the stem sequences form a stem structure in the RNA output. Put another way, the aptamer region is in the center of a palindromic or substantially palindromic sequence, such that the aptamer region forms a loop and the flanking sequence form a stem in the transcribed RNA output (FIG. 3). The stem sequences comprise any sequence or any length that prevents unwanted secondary structure formation between the aptamer and output region in the corresponding RNA output. In some embodiments, the stem sequences are each 2-20 bases in length. In some embodiments, the stem sequences are each 5-7 bases in length. In select embodiments, the stem sequences are each 6 bases in length. In some embodiments, the stem sequences are at least partially double stranded in the transcription template.

The transcription template comprises a promoter region. The promoter can be any promoter for any RNA polymerase. Consideration should be given to ensuring the promoter region of the transcription template is compatible with the desired RNA polymerase and vice versa. In some embodiments, the promoter region of the transcription template is double stranded.

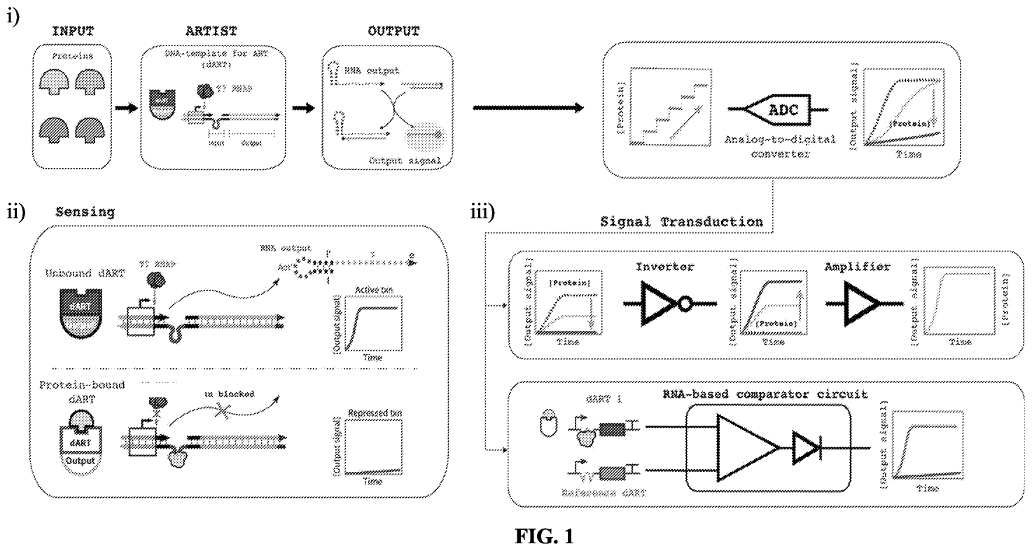

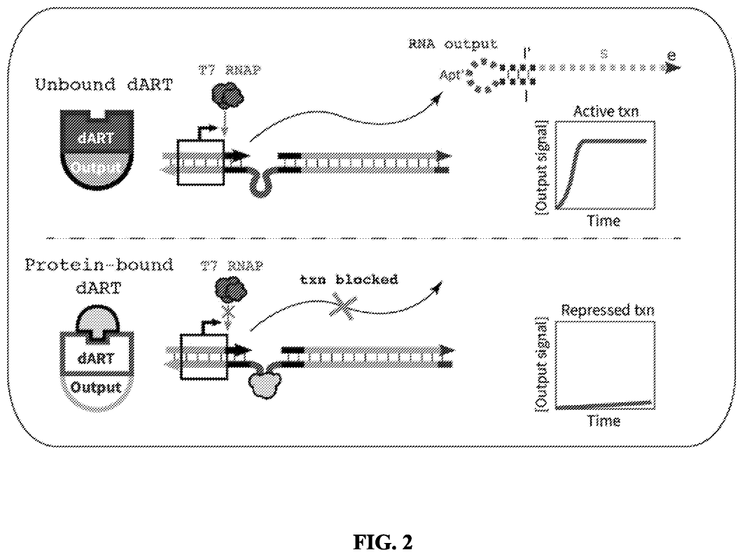

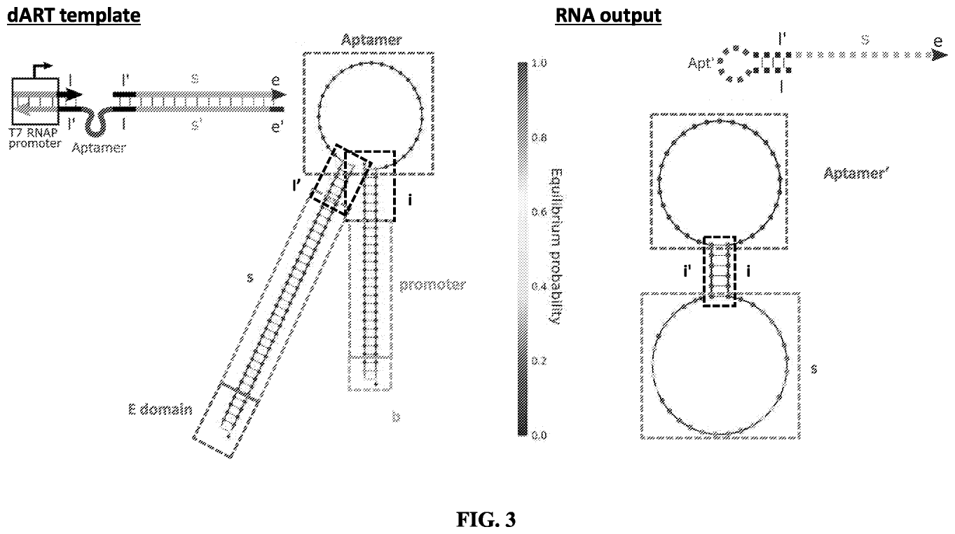

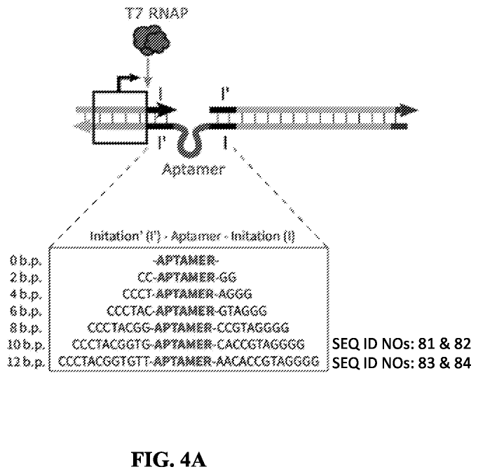

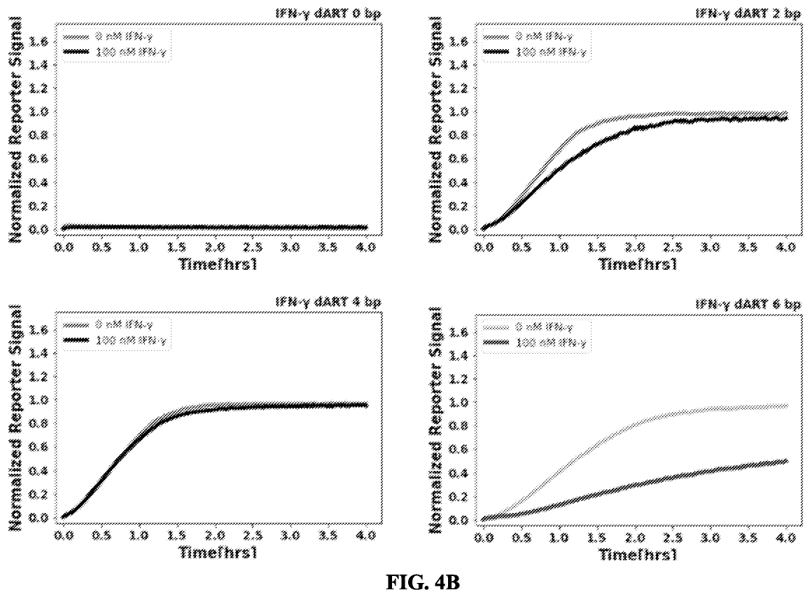

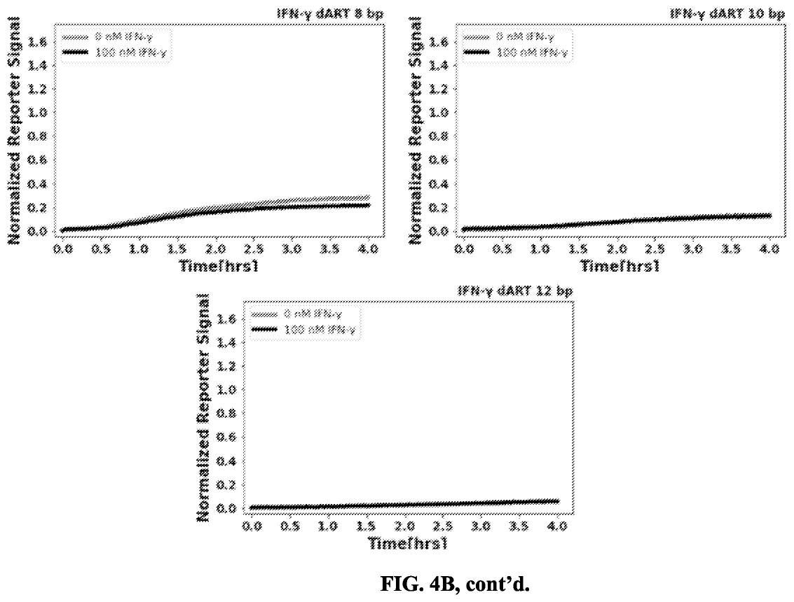

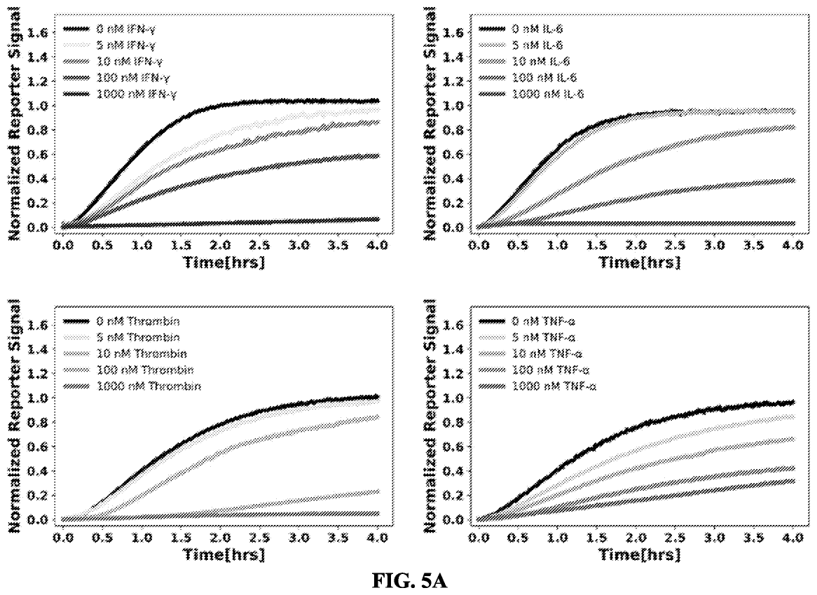

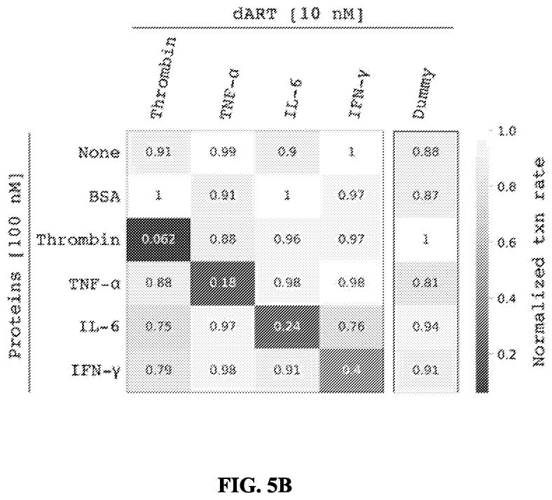

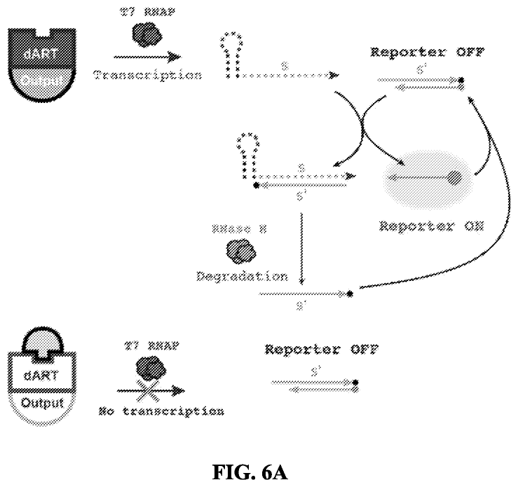

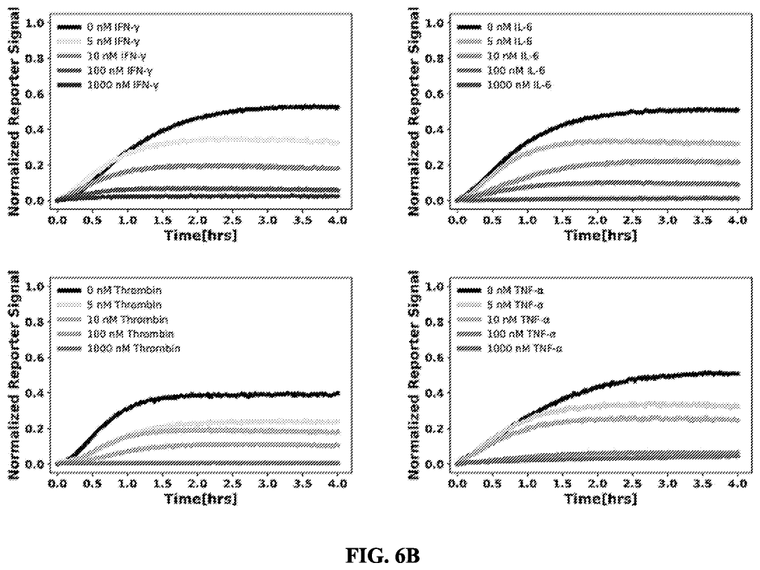

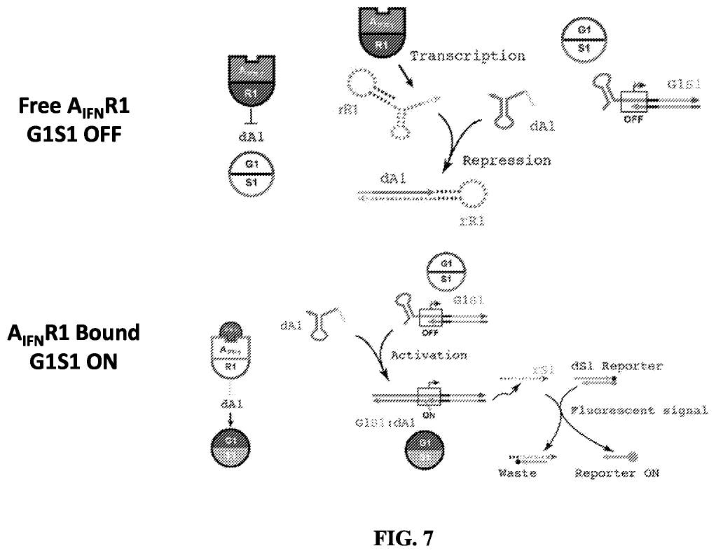

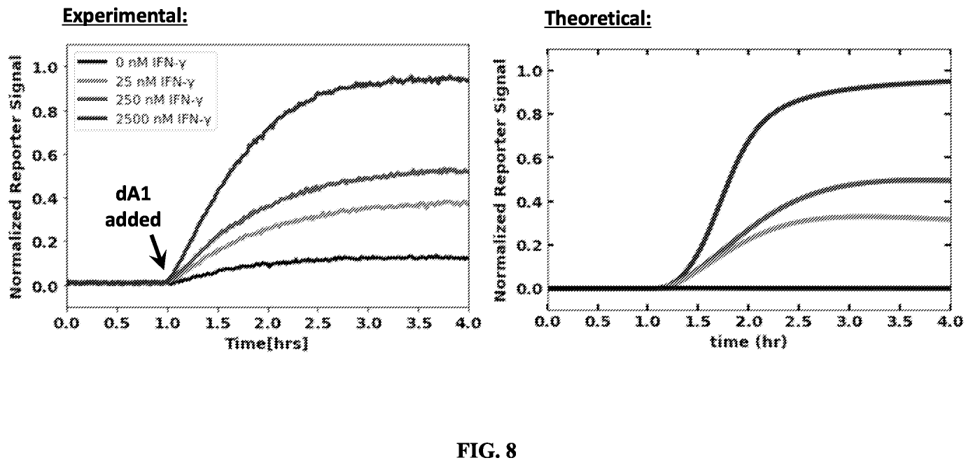

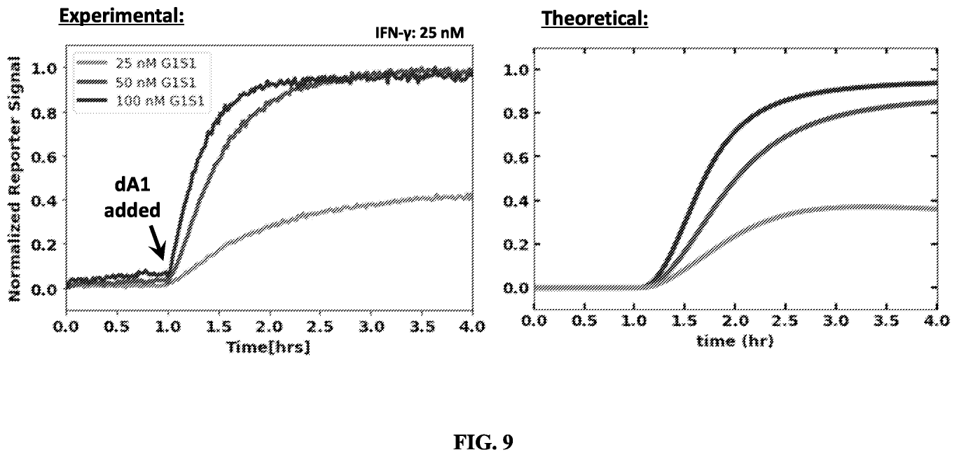

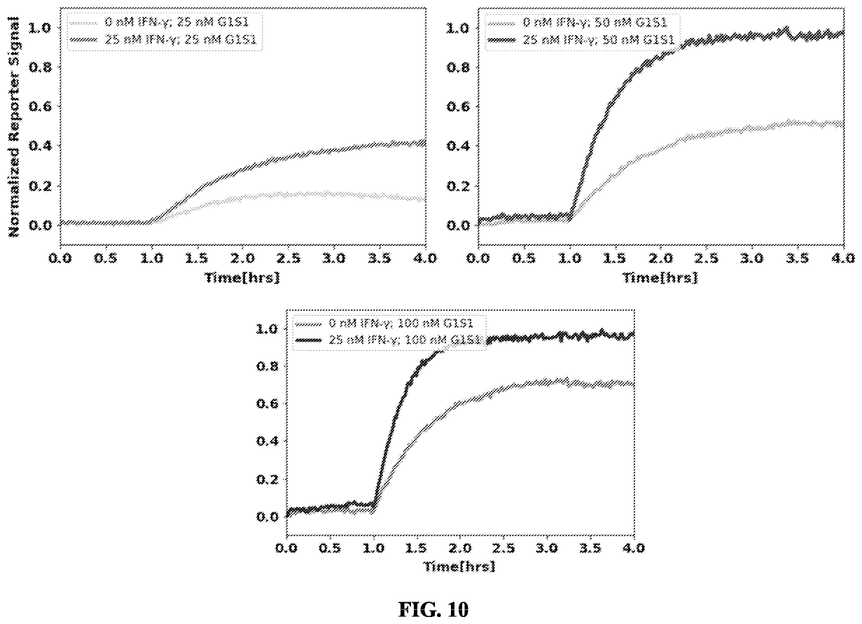

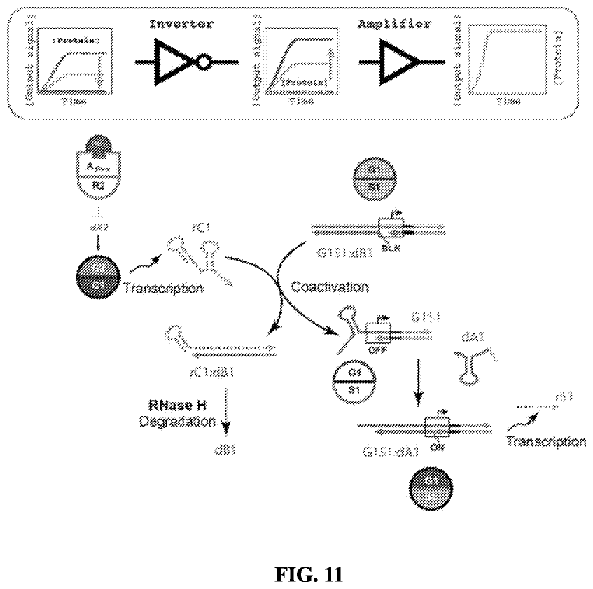

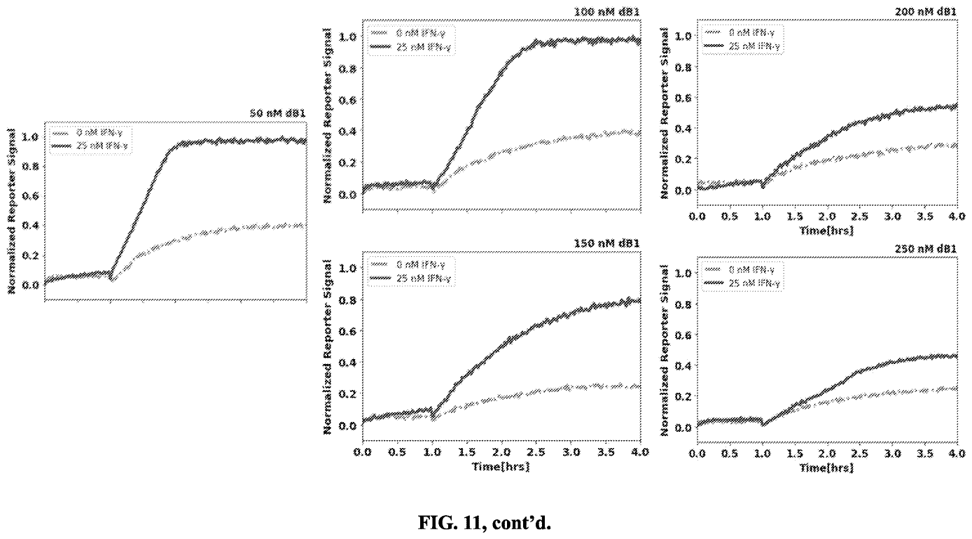

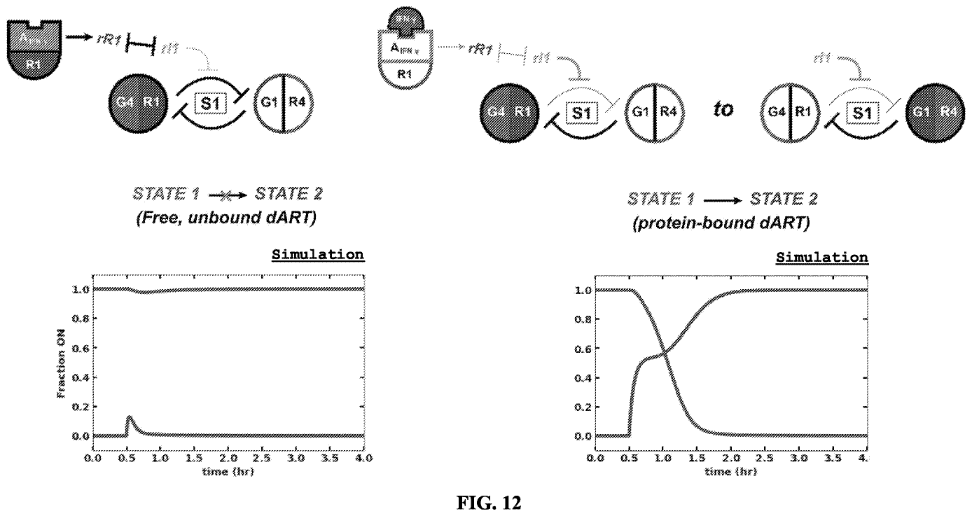

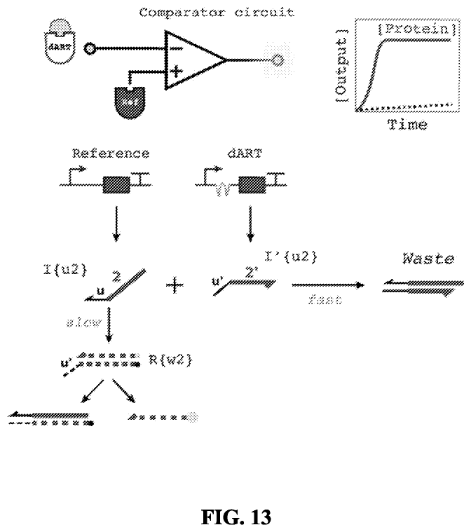

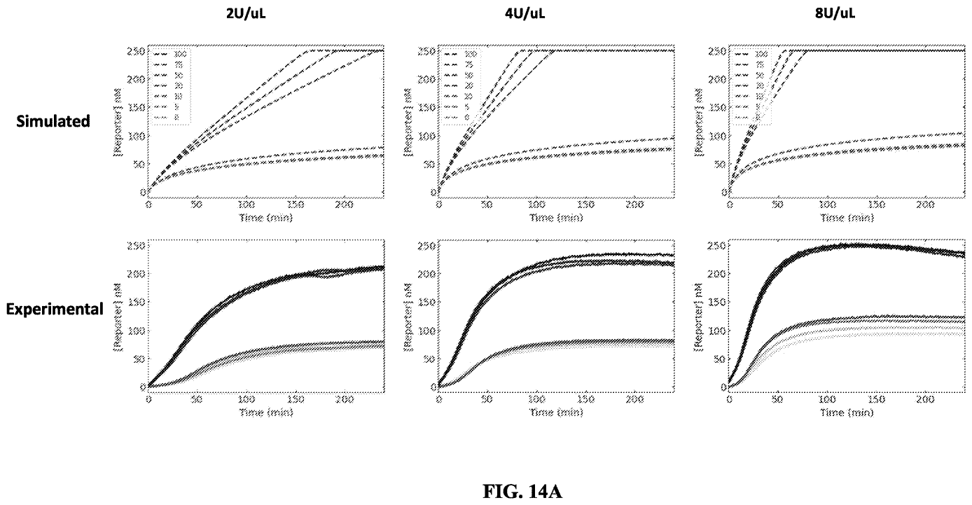

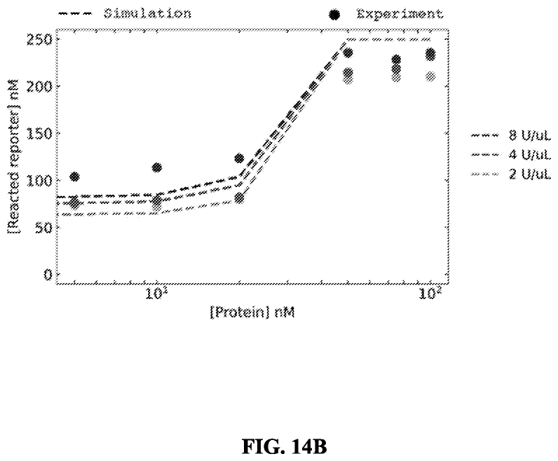

In some embodiments, the systems further comprise an RNA polymerase which is configured to transcribe output from the transcription template. As shown in FIG. 1, the system, in the absence of a molecule of interest, facilitates transcription from the template and produces the RNA output. Upon binding of the molecule of interest in the aptamer region, the RNA polymerase is blocked from transcribing the output region of the transcription template and production of the RNA output is repressed. Accordingly, the system detects the molecule(s) of interest by the decrease in the production of RNA output.

RNA polymerases for use in the disclosed systems can be any polymerase that supports in vitro transcription from the transcription template. Examples of suitable polymerases include E. coli RNA Polymerase, T3 RNA Polymerase, T7 RNA Polymerase SP6 RNA Polymerase, and the like. Phage RNA polymerases, such as T3, T7 and SP6 RNA Polymerases, may be particularly useful due to the small size of their promoters (e.g., 17-20 nucleotides in length).

The output region of the transcription template is not limited by sequence, structure, or lengths. Generally, the output sequence can be tailored to the methods by which the RNA output may be detected or for use with downstream applications, such as in vitro networks of chemical and material regulation. For multiplex systems which comprise more than one first transcription template to detect more than one molecule of interest, the RNA output may be designed to be detected as a group (e.g., output region sequences are identical or substantially identical) or individually (e.g., output region sequences are each substantially different). In some embodiments, the output region of the transcription template is double stranded.

The system finds use in synthesizing RNA molecules and producing RNA compositions. Thus, the application further provides methods for synthesizing RNA molecules under the control or as a result of the level of a molecule of interest and methods for using the synthesized RNA molecules. As such, the system may be integrated with other methods, components, or systems which operate, are linked through, or utilize an RNA molecule or RNA composition, e.g., the RNA output. The present system is not limited to the particular methods, components, or systems in which it may provide a means to produce the RNA of interest.

The system may be integrated with components to measure the RNA output(s), or components which utilize the RNA output(s) to perpetuate or transduce the RNA output. For example, the system may be integrated with other in vitro transcription networks and circuits or chemical reactions, e.g., binding or other catalytic reactions. See, for example, Schaffter et al., Nat. Chem. 14, 1224-1232 (2022), Schaffter, S. W., Schulman, R. Nat. Chem. 11, 829-838 (2019), and Schaffter S W, Strychalski E A. Sci Adv. 2022 Mar. 25; 8(12):eab14354, each of which is incorporated herein by reference in its entirely.

The system may be integrated with hydrogels. For example, the disclosed system may be utilized to produce RNA molecules that have the capacity to form a hydrogel or RNA molecules which influence the properties of a hydrogel being formed or pre-formed. The hydrogels may be utilized in therapeutic applications or methods for regulating other biochemical processes. The hydrogels can be integrated with synthetic biology components or systems.

The system, or hydrogels thereof and RNA output can find use in therapeutic applications or methods for regulating other biochemical processes in a cell or subject (e.g., regulating cell signaling by binding to signaling molecules (e.g., receptors and ligands). The RNA output may be therapeutic RNA molecules (e.g., mRNA molecules encoding therapeutic proteins), non-coding immunostimulatory RNA molecules, or other non-coding or signaling RNAs such as microRNAs, small interfering RNAs, CRISPR guide RNAs, small nucleolar RNAs, riboswitches, trans-acting RNAs, and long non-coding RNAs and be part of system which generates these therapeutic RNA molecules in response to the level of a molecule of interest. The RNA output may interact with a responsive therapeutic, living cell or components thereof, or a responsive nanomaterial, such as an assembly of gold nanoparticles.

Detecting an RNA molecule is generally done by either sequencing or reacting the molecule with another agent or moiety. Any method known in the art can be used to detect the RNA output from the systems described herein. For example, reporter agents which are configured to detect the RNA output, fluorogenic aptamers, RNA reactions, optionally with another reagent or compound, to produce a readout including but not limited to hydrogel shape change, sequencing methods (e.g., microarray, next-generation, nanopore sequencing approaches), reverse transcription reactions, protein expression systems, RNA or Cas protein cleavage, color change, change in fluorescence resulting in reactions with the RNA, and nanostructure assembly.

In some embodiments, the system further comprises a nucleic acid reporter. The nucleic acid reporter may be single or double stranded, and may be DNA or RNA. The nucleic acid reporter is configured to detect the RNA output. Preferably, the nucleic acid reporter comprises a sequence complementary to at least part of the RNA output. For example, in some embodiments, the nucleic acid reporter is double stranded and comprises one strand which is complementary to at least a portion of the RNA output.

In some embodiments, the nucleic acid reporter comprises at least one detection moiety. The at least one detection moiety is configured in such a way as to monitor of the formation of the RNA output-nucleic acid reporter double stranded nucleic acid. For example, the detection moiety may be on the non-complementary strand of a double stranded nucleic acid reporter, such that it is detectable, after binding of the RNA output to the complementary strand nucleic acid reporter. Alternatively, the detection moiety may be on the complementary strand but is no longer masked or shielded (e.g., by a quencher) when hybridizing to the RNA output.

As used herein, the term “detection moiety” refers to any compound or combination of compounds that may be attached or otherwise associated with a molecule so that the molecule can be detected directly or indirectly by utilizing the detection moiety. A detection moiety can be a radioisotope (e.g., carbon, phosphorus, sulfur, tritium, etc.), a mass isotope (e.g., H², C¹³ or N¹⁵), a dye or fluorophore (e.g., cyanine, fluorescein or coumarin), a hapten (e.g., biotin) or any other agent that can be detected directly or indirectly.

In some embodiments, the system further comprises an RNA output inversion module. The RNA output inversion module allows detection of the repression of RNA output as an inverted or positively influenced signal. Therefore, instead of directly detecting the decrease of RNA output, the inversion module transduces the RNA output into a read-out or signal which increases with decreasing RNA output production or, accordingly, with the presence of the molecule of interest.

In certain embodiments, the RNA output inversion module comprises a second nucleic acid transcription template comprising a partially single stranded promoter site and an output region which encodes a second RNA output and a first activator strand which is complementary to the single stranded portion of the promoter site and the first RNA output. The activator strand completes the promoter site, such that when the first activator strand binds to the partially single stranded promoter site it facilitates transcription from the second nucleic acid transcription template.

The first RNA output regulates transcription of the second transcription template by controlling the availability of the activator strand through nucleic acid hybridization and strand displacement. As such, the sequences of at least part of the promoter site of the second transcription template and the first activator strand can be designed to be complementary nucleic acid sequences to each other and, in the case of the first activator strand, to the RNA output from the first transcription template. As such, in the presence of the molecule of interest, the product of the second RNA output is increased.

In some embodiments, the system further comprises an RNA output amplification module. An RNA output amplification module facilitates amplification of the output RNA signal or the inverted signal. An amplification module may provide increased signal to noise, increased resolution, particularly at lower concentrations of the molecule of interest, or a combination thereof.

In certain embodiments, the RNA output amplification module comprises a third nucleic acid transcription template comprising a partially single stranded promoter site and a sequence encoding a third RNA output; a second activator strand which is complementary to the partially single stranded promoter site on the third nucleic acid transcription template; and a blocker strand complementary to the second RNA output and the single stranded promoter site on the third nucleic acid transcription template. The blocker strand is configured to prevent transcription from the nucleic acid third transcription template. Similar to the first activation strand, the second activation strand completes the promoter site, such that it facilitates transcription from the third nucleic acid transcription template.

The RNA second output regulates transcription of the third transcription template by controlling the availability of the blocker strand to bind to the incomplete promoter of the third transcription template through nucleic acid hybridization and strand displacement. The sequences of at least part of the promoter site of the third transcription template, the blocker strand, and the second RNA output, can be designed can be designed such that the blocker strand is complementary to both the second RNA output and the promoter site of the third transcription template. Thus, when the second RNA output increases the third transcription template is available for binding by the second activator strand to produce an amplified output in larger quantities than either the first or second RNA outputs. As such, in the presence of the molecule of interest, the signal from the first and/or second RNA output is amplified.

In some embodiments, the system further comprises a self-calibration module. A self-calibration module provides an internal reference for the RNA output from the any of the first, second, or third transcription templates. In some embodiments, the self-calibration module comprises a reference nucleic acid transcription template lacking a single stranded aptamer domain. The reference transcription template and the transcription template (e.g., the first transcription template) are both transcribed in the absence of the molecule of interest. The reference transcription template and the transcription template (e.g., the first transcription template) are configured to be transcribed at the same rate. For example, the reference transcription template and the transcription template (e.g., the first transcription template) may have the same promoter sequence, the same length of the initiation sequence (i domain after the promoter sequence, as shown in FIG. 3), and presence of similar secondary structures (e.g., a G-quadruplex) after the i domain. When the molecule of interest is present, the reference transcription template continues to transcribe at the same rate as previously, but transcription of the first transcription template is repressed due to the binding of the molecule of interest to the aptamer region of the first transcription template.

In some embodiments, the RNA output from the coordinating transcription template and the RNA output from the reference transcription template comprise complementary sequences. Accordingly, the output regions of the coordinating transcription template and the reference transcription template are similarly complementary. In the absence of the molecule of interest, the RNA output from each of the reference transcription template and the coordinating transcription template bind to each other and are degraded.

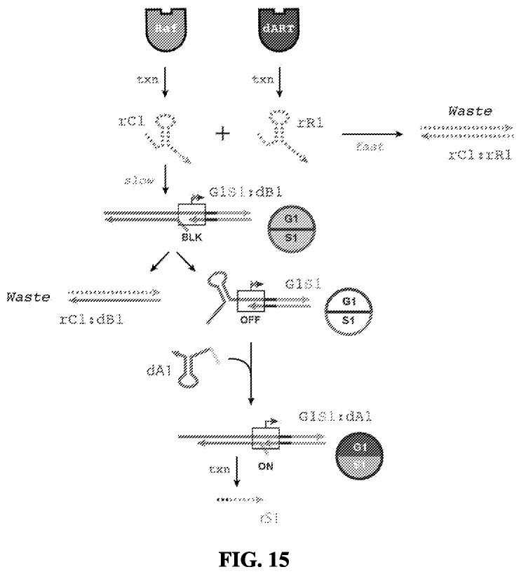

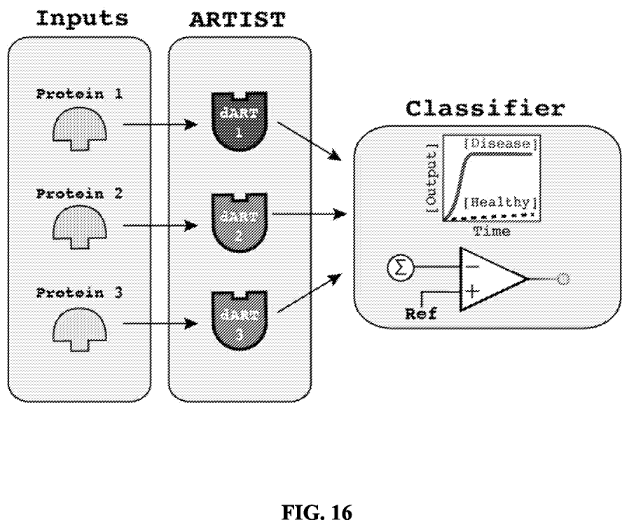

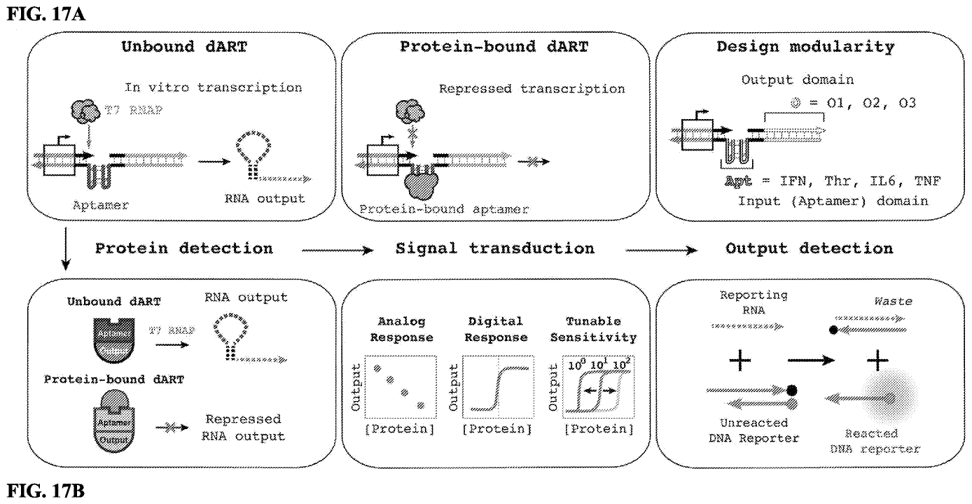

The self-calibration module may further comprise components to measure the reference RNA output. In some embodiments, the self-calibration module further comprises a reference reporter nucleic acid. Similarly, to the reporter nucleic acid described above for the first transcription template, the reference reporter nucleic acid shares at least partial complementarity with the reference RNA output. The reference reporter nucleic acid may also comprise at least one detection moiety. In some embodiments, the reference RNA output may be transduced using amplifier or inversion module as described above for the first RNA output, as shown in FIG. 15.

In some embodiments, the systems are embedded in a hydrogel. Hydrogels are a type of gel in which water-swellable polymeric matrices that can absorb a substantial amount of water in a three-dimensional network of macromolecules (e.g., hydrophilic polymer chains) that form the matrix are held together by covalent or noncovalent crosslinks. The hydrogel matrix may comprise peptides (e.g., oligopeptides or polypeptides), non-peptide monomers (e.g., (meth)acrylamide monomers, (meth)acrylate monomers, N-vinyl lactam monomers, acrylamide, methacrylamide, N,N-dimethyl acrylamide (DMA), N-isopropyl acrylamide (NIPAM), hydroxyethyl acrylate (HEA), poly(ethylene glycol) methyl ether acrylate (PEGA), poly(ethylene glycol) methyl ether methacrylate (PEGMA), 2-hydroxypropyl methacrylamide (HPMA), 2-hydroxyethyl methacrylate (HEMA), 2-methoxyethyl acrylate (MEA), methacrylic acid (MAA), 4-acryloylmorpholine (ACMO), dimethyl vinylphosphonate (DEVP), N-vinylcaprolactam (NVCL), N-vinyl pyrrolidone (NVP), and combinations thereof).

In some embodiments, at least one of the nucleic acid transcription template and the nucleic acid reporter are linked to a matrix molecule of the hydrogel. In some embodiments, the nucleic acid transcription template is linked to a matrix molecule of the hydrogel. In some embodiments, the nucleic acid reporter are linked to a matrix molecule of the hydrogel. Accordingly, one or both of the nucleic acid transcription template and the nucleic acid reporter may comprise a reactive moiety (e.g., a crosslinkable moiety) to generate the link to the hydrogel matrix molecule. Reactive moieties are known in the art and depend on the particular hydrogel matrix components, the nature of the bond being formed between the hydrogel and system component (e.g., nucleic acid transcription template, nucleic acid reporter), and, if applicable, crosslinking reagents.

Additionally or alternatively, at least one or all of the following may be linked to the hydrogel matrix and comprise a reactive moiety: the second nucleic acid transcription template, first nucleic acid activator strand, third nucleic acid transcription template, second nucleic acid activator strand, nucleic acid blocker strand, reference nucleic acid transcription template.

RNA signals can be degraded by enzymes, such as ribonucleases (RNases), which when utilized in systems in which RNA is produced, can be coupled to establish turn-over or steady-state conditions, as desired. In some embodiments, the systems disclosed herein further comprise an RNase. In certain embodiments, the disclosed systems comprise a RNase which degrades double stranded RNAs or RNA/DNA duplexes. In select embodiments, the disclosed systems comprise an RNaseH.

The system may further comprise other components useful in in vitro transcription reactions, including but not limited to, nucleoside triphosphates (NTPs) (e.g., naturally occurring, synthetic, and/or modified NTPs, e.g., ATP, UTP, GTP, and CTP), buffers, cofactors (e.g., magnesium, manganese), detergents (e.g., Triton X-100), reducing agents (e.g., DTT, TCEP), small molecule additives (e.g., spermidine), and enzymatic additives (e.g., inorganic phosphatase orpyrophosphatase)

The system may further comprise an RNA detection component. The RNA detection component may comprise any reagents or instrumentation which facilitate the measurement of RNA. For example, the system may further comprise: reagents and equipment necessary for sequencing RNA (e.g., oligonucleotide primers, thermocyclers, or incubators), spectrophotometers, fluorometers, microfluidic devices, and the like.

In some embodiments, the system further comprises a sample. As used herein, the term “sample” is used in its broadest sense. In one sense, it is meant to include a specimen obtained from any source, including biological and environmental samples. Biological samples may be obtained from animals (including humans) and encompass fluids, solids, tissues, and gases. Biological samples can be obtained from a subject using routine techniques known to those skilled in the art. Such examples are not however to be construed as limiting the sample types. Preferably, a sample is a fluid sample such as a liquid sample. Examples of liquid samples that may be assayed include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof (e.g., a suspension of lysed or unlysed cells in a buffer or solution). Samples can comprise biological materials, such as cells, microbes, organelles, and biochemical complexes. Liquid samples can be made from solid, semisolid, or highly viscous materials, such as soils, fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample.

Samples may be used directly as obtained from the source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

### 3. METHODS

The present disclosure provides systems which are useful in methods of detecting molecules of interest. As such, also disclosed herein are methods for detecting at least one molecule. The methods comprise incubating a sample comprising at least one molecule with a system as described herein. In some embodiments, the methods comprise incubating the sample with a system comprising two or more first transcription templates, such that the methods detect two or more molecules in the sample. In some embodiments, the molecules are biomolecules. In some embodiments, the biomolecules are biomarkers. In some embodiments, the biomolecules are proteins.

The incubation may be done under conditions to promote transcription. For example, the conditions may include incubation in a reaction mixture including any or all of: nucleoside triphosphates (NTPs) (e.g., naturally occurring, synthetic, and/or modified NTPs, e.g., ATP, UTP, GTP, and CTP), buffers, cofactors (e.g., magnesium, manganese), detergents (e.g., Triton X-100), reducing agents (e.g., DTT, TCEP), small molecule additives (e.g., spermidine), and enzymatic additives (e.g., inorganic phosphatase or pyrophosphatase).

Additionally, the incubation may be completed at a period of time and temperature to facilitate transcription by the RNA polymerase. Time and temperature conditions may be influenced by the RNA polymerase being used in the system. For example, a temperature between 20° C. and 37° C. for 5 minutes to 4 hours for may be suitable for RNA production using the disclosed systems.

The methods further comprise measuring the at least one first RNA output or a signal corresponding to the at least one first RNA output. The signal corresponding to the at least one first RNA output may include any signal resulting from transducing the first RNA output or signal thereof. For example, the signal corresponding to the at least one first RNA output may be the second RNA output, the third RNA output and/or the reference RNA output.

The methods are not limited by the means by with the RNA outputs are measured. Any method for detecting or measuring RNA can be used. Such methods are known to those in the art, and include, but are not limited to the use of amplification-based methods (e.g., PCR, RT-PCR, Q-PCR), array-based technologies, RNA-seq, gel separation (e.g., agarose or acrylamide gel) methods, incorporation of radioactive ribonucleotides, and hybridization based methods. The measuring can be done by using polynucleotides which are specific and/or selective for the RNA outputs through hybridization, such as the reporter nucleic acids described in the systems above.

Detection or measurement of the levels or concentrations of molecules (e.g., biomolecules) finds use in methods of determining the presence or risk of a disease or disorder for a subject. Accordingly, in some embodiments, the methods further comprise determining the presence, status, or risk of a disease or disorder based on the RNA outputs, and accordingly, the detection or level of a biomolecule or biomarker, e.g., based on range(s) or levels of each individual biomarker being indicative of the presence, status, or risk of a disease or disorder. In some embodiments, the method comprises generating a composite score based on the detection or level of two or more biomolecules or biomarker. In some embodiments, a clinician or other medical personnel can compare the composite score for the subject with a scoring metric. The scoring metric may provide values, ranges or cut-offs which relate to risk levels (e.g., very high, high, moderate, low, or very low) or probabilities for one or more diseases or disorders.

### 5. KITS

The disclosure also provides a kit for detecting or measuring at least one molecule in a sample. The kit comprises transcription template or a system as described herein. In some embodiments, the kit further comprises any or all of: nucleoside triphosphates (NTPs) (e.g., naturally occurring, synthetic, and/or modified NTPs, e.g., ATP, UTP, GTP, and CTP), buffers, cofactors (e.g., magnesium, manganese), detergents (e.g., Triton X-100), reducing agents (e.g., DTT, TCEP), small molecule additives (e.g., spermidine), and enzymatic additives (e.g., inorganic phosphatase or pyrophosphatase). The kit may further comprise reagents for detecting the RNA output from the transcription templates disclosed herein.

Individual member components of the kits may be physically packaged together or separately. The components of the kit may be provided in bulk packages (e.g., multi-use packages) or single-use packages. The kits can also comprise instructions for using the components of the kit. For example, the kit may comprise instructions for using the transcription template or system as described herein. The instructions are relevant materials or methodologies pertaining to the kit. The materials may include any combination of the following: background information, list of components and their availability information (purchase information, etc.), brief or detailed protocols for using the compositions, troubleshooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods, compositions or systems disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like.

### 6. EXAMPLES

### Materials and Methods

### Materials

O4_f, O1′_q, O2′_q, O3′_q, and O4′_q were purchased from Integrated DNA Technologies (IDT) with HPLC purification. All other oligonucleotides were purchased under standard desalting conditions. Triphosphates (NTPs) were purchased from ThermoFisher Scientific (EP0113). T7 RNAP was purchased in bulk (300,000 U) from Cellscript (200 U μl⁻¹, C-T7300K) as well as from ThermoFisher Scientific (200 U μl⁻¹, EP0113). NEB RNAPol reaction buffer (M0251S; 10×) and yeast inorganic pyrophosphatase (YIPP; M2403S; 0.1 U μl⁻¹) were purchased from New England Biolabs (NEB). RNase H was purchased from ThermoFisher Scientific (EN0201; 5 U μl⁻¹).

Recombinant Human IFN-γ (285-IF), IL-6 (206-IL), TNF-α (210-TA), and VEGF 165 (293-VE) were all purchased from R&D Systems Inc. (U.S.A.) in lyophilized form. Recombinant Human IFN-γ was reconstituted in sterile, deionized water, whereas Recombinant Human IL-6, TNF-α, and VEGF were all reconstituted in sterile PBS containing 0.1% BSA. Human α-thrombin (HCT0020) was purchased from Haematologic Technologies, Inc. (Essex, VT) and dissolved in 50% H₂O/glycerol.

Template preparation To prepare a dART template, Apt-T7p-nt was annealed with the output non-template strand (Apt-Rep2b-nt) and a template strand that contains the aptamer domain of choice (e.g., Apt-Rep2b-IFN-t, Apt-Rep2b-Thr-t, Apt-Rep2b-IL6-t, Apt-Rep2b-TNF-t, Apt-Rep2b-VEGF-t). For example, Apt-T7p-nt, Apt-Rep2b-nt, and Apt-Rep2b-IFN-t were diluted in a standard 200 μL PCR tube. 1 μM per strand in 1×NEB RNAPol reaction buffer (catalogue number M0251S) was supplemented with 100 mM KCl. The 1×NEB RNAPol reaction buffer contained 50 mM Tris-HCl, 100 mM NaCl, 20 mM 3-ME, 1 mM EDTA, 50% Glycerol, 0.1% (w/v) Triton® X-100, pH 7.9. To anneal, the mixture was heated to 90° C. for 5 minutes, then slowly cooled to 20° C. at a rate of −1° C./min. This annealing process yielded a mixture of 1 μM dART template. This was repeated for all other dARTs with different aptamer domains (Apt-Rep2b-Thr-t, Apt-Rep2b-IL6-t, Apt-Rep2b-TNF-t, Apt-Rep2b-VEGF-t).

dART annealing and preparation dARTs were assembled by annealing their three strands, a promoter non-template strand (Prom-dART-nt), a non-template strand encoding the output sequence of choice (e.g., O1-dART-nt, O2-dART-nt, O3-dART-nt) and a template strand that contains the aptamer domain of choice that is complementary to the two non-template strands (e.g., IFN-O1-dART-t, Thr-O1-dART-t, IL6-O1-dART-t, TNF-O1-dART-t) at equimolar concentrations. As an example, Prom-dART-nt, O1-dART-nt, and IFN-O1-dART-t were combined in a standard 200 μL PCR tube (VMR; 20170-010) at concentrations of 1 μM per strand in 1×NEB RNAPol reaction buffer supplemented with KCl to a final concentration of 100 mM. To anneal, mixtures were heated to 90° C., incubated for 5 min, then cooled to 20° C. at a rate of 1° C. min⁻¹.

DNA Reporter preparation DNA reporters were utilized to keep track of how much RNA has been produced from the reactions. The DNA reporters were prepared by diluting the fluorophore-modified DNA strand (Rep2b-F) with its partially complementary quencher-modified DNA strand (Rep2b-Q) at a concentration of 10 μM per strand in 1×NEB RNAPol reaction buffer. The reporter mixture was heated to 90° C., incubated for 5 min, then cooled to 20° C. at a rate of 1° C. min⁻¹. The RNA produced from an active dART was designed to hybridize with the quencher-modified strand via toe-mediated strand displacement reaction, such that the resulting single-stranded fluorophore-modified DNA was tracked by fluorescence output.

Amplified comparator annealing and preparation Genelet initially in a blocked state (G1O4:B1) was prepared by mixing G1O4-nt, O4-t, and B1 together in 1×NEB RNAPol reaction buffer at equimolar concentrations. The genelet mixture was heated to 90° C., incubated for 5 min, then cooled to 20° C. at a rate of 1° C. min⁻¹.

Reaction conditions and data acquisition Reactions were all conducted at 37° C. in 1×NEB RNAPol reaction buffer supplemented with 100 mM KCl and NTPs (ATP, UTP, CTP, GTP; final concentration, 2 mM each unless otherwise stated). 100 mM KCl was included to promote proper folding of the aptamer domain into a G-quadruplex structure. In addition to T7 RNA polymerase, YIPP was also included in reactions (1.35×10⁻³ U μl⁻¹) to extend the duration of the transcription reactions. The reaction conditions mentioned above are referred to as ARTIST reaction conditions. For reactions in which steady-state production of RNA was induced, RNase H was added into the reaction (2×10⁻³ U μl⁻¹) to degrade the RNA bound to the quencher strand of the DNA reporter. The total volume of the reaction was set at 25 mL for all assays.

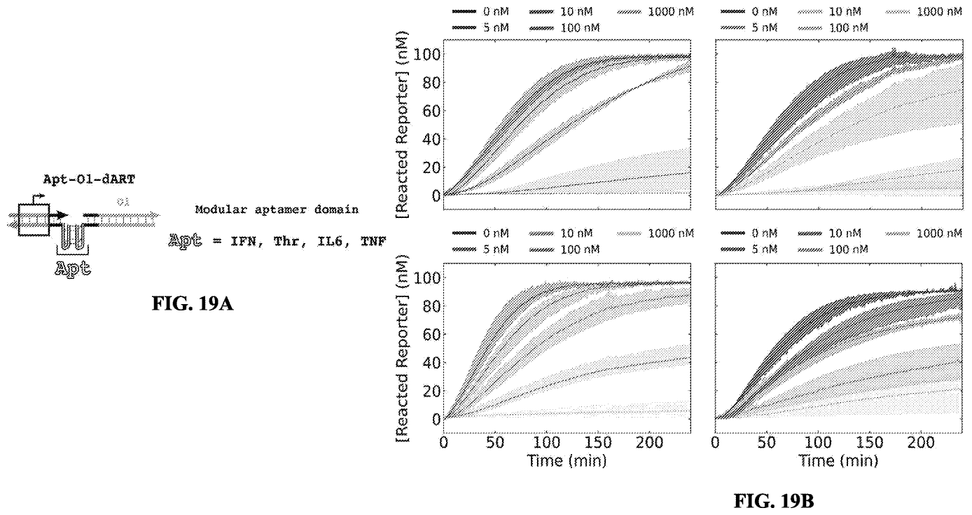

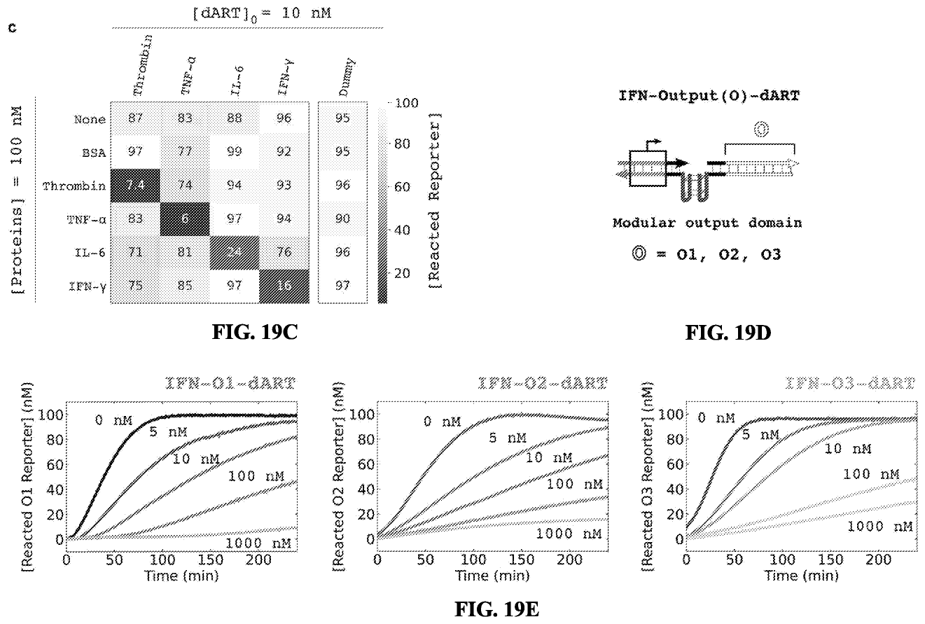

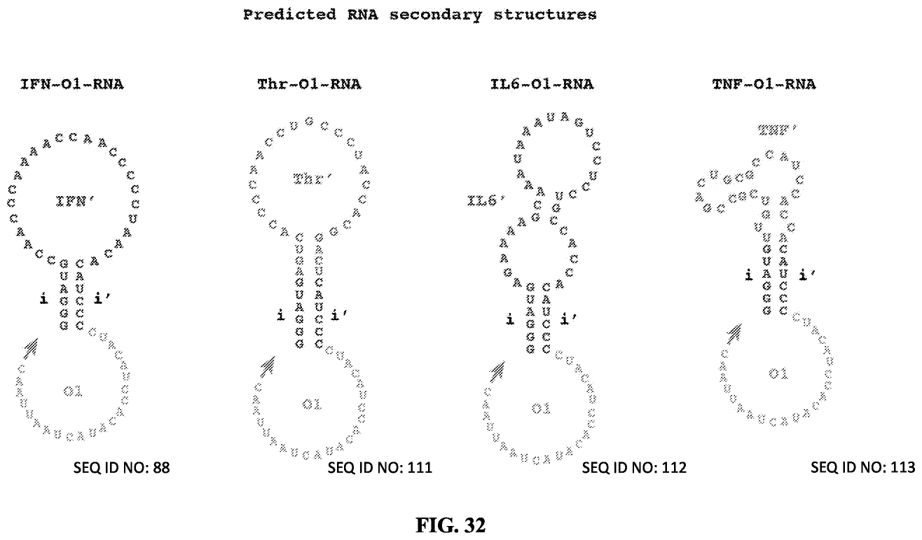

To perform experiments, solutions containing dART templates under ARTIST reaction condition mentioned were first added to wells of a 384-well plate. Proteins were then added at the concentrations described set at a volume of 0.5 μL and incubated for 30 min to 60 min at room temperature. In FIG. 19B, 0.5 μL of sterile PBS containing 0.1% BSA were also added into the assays with 10 nM of IL6-O1-dART or TNF-O1-dART reacting with 0 nM of IL-6 or TNF-α, respectively. This was done to ensure equal salt concentrations from the PBS buffer. Similarly in FIG. 32, 0.5 μL of sterile PBS containing 0.1% BSA was added into all assays that did not have 100 nM of IL-6 or TNF-α.

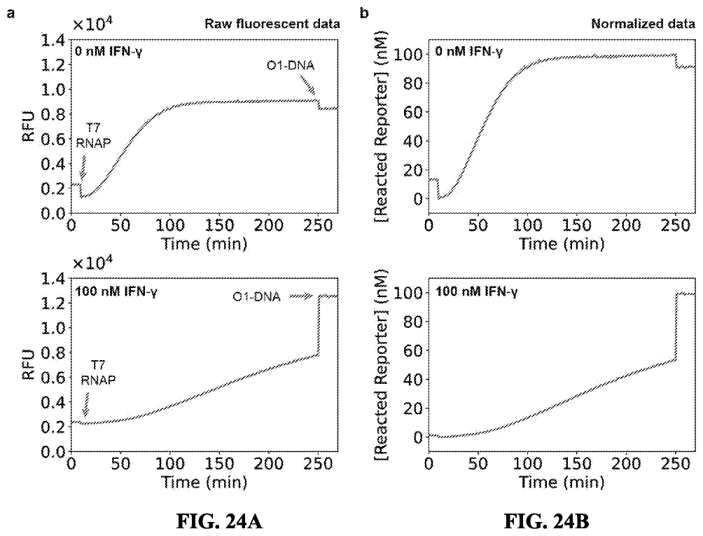

Data acquisition Fluorescence readings were then taken for 10 min to 25 mins to measure minimum fluorescence values before T7 RNAP, and YIPP were added to initiate the reactions (FIG. 24A). At the end of the experiments, 0.5 μL of a DNA strand fully complementary to o1′_q or o4′_q was mixed into each assay at a final concentration of 2.5 μM to obtain a maximum O1 or O4 DNA reporter fluorescence intensity. Fluorescence data were then normalized using Eqn 1 as follows (FIG. 24B):

$$[ Reacted Reporter ] = ( Total [ DNA Reporter ] ) * \frac{Fluorescence - Min ( Fluorescence )}{Max ( Fluorescence ) - Min ( Fluorescence )} \qquad ( 1 )$$

All kinetic data were obtained using either a BioTek Synergy H1 or Cytation 5 plate reader (Agilent Technologies). HEX was measured with an excitation peak of 533 nm and an emission peak of 559 nm with a gain of 80 to 100 to ensure fluorescence values were within the linear range of detection. Cy3 was measured with excitation peak of 555 nm and emission peak of 569 nm with a gain 60 to 100 to ensure fluorescence values were within the linear range of detection. FAM was measured with excitation peak of 487 nm and emission peak of 527 nm with a gain of 60. Fluorescence measurements were taken every minute during reactions. For independent replicates, all experiments were conducted using the same instrument, reagents, and experimental conditions on two or three separate days.

Inverter circuit Transcriptional elements termed G1S1 and dA1 were derived from Schaffter et al. (Nat. Chem. 14, 1224-1232 (2022)). Initially, 25 nM of dART-R1 template, 25 nM of G1S1, 250 nM of S1 DNA reporter were mixed in the ARTIST reaction conditions in a well of a 384-well plate. IFN-γ with varying concentrations (0-2500 nM) was added into the mixture. Fluorescence readings were first taken for 10 to 25 mins before the enzymes (T7 RNAP, YIPP, RNase H) were added to initiate the reactions. 60 minutes after adding the enzyme mix, 50 nM of dA1 was added into the assay, and fluorescence of the S1 reporter was measured. At the end of the experiments, 0.5 mL of the DNA version for rS1 concentrated at 2.5 μM was added to obtain an internal maximum DNA reporter fluorescence value.

Integrated inverter and amplifier circuit Transcriptional elements termed G1S1, G2C1, dA1, dB1, and dA2 were derived from Schaffter et al (Nat. Chem. 14, 1224-1232 (2022)). Initially, 25 nM of dART-R1 template, 50 nM of G2C1, 50 nM of G1S1, 50 nM of dB1 250 nM of S1 DNA reporter were mixed in the ARTIST reaction condition in a well of a 384-well plate. IFN-γ with varying concentrations (0-2500 nM) are added into the mixture. Fluorescence readings were first taken for 10 to 25 mins before the enzymes (T7 RNAP, YIPP, RNase H) were added to initiate the reactions. 60 minutes after adding the enzyme mix, dA1 and dA2 were added into the assay concentrated at 50 nM per strand. Fluorescence of the S1 reporter was measured using the plate reader. At the end of the experiments, 0.5 mL of the DNA version for rS1 concentrated at 2.5 μM was added to obtain an internal maximum DNA reporter fluorescence value.

Comparator circuit Initially, 25 nM of Ref-Rep2b template and 250 nM of Rep2b DNA reporter were mixed in the ARTIST reaction conditions in a well of a 384-well plate. For experiments that only vary the dART template concentration, a range of 0-100 nM of the dART-IFN-Rep2bc template was used. For experiments that involve IFN-γ, the dART-IFN-Rep2bc template concentration was fixed at 50 nM, and 0-100 nM of IFN-γ was mixed into our reactions. Fluorescence readings were first taken for 10 to 25 mins before T7 RNAP and YIPP were added to initiate the reactions. Herein, the concentration of T7 RNAP was varied to consider a variation of its activity (2-8 U μl⁻¹) in terms of transcription. At the end of the experiments, 0.5 mL of dART Rep2b-nt concentrated at 2.5 μM was mixed into each assay to obtain an internal maximum DNA reporter fluorescence value.

Inverter circuit simulation variables Simulation for the ARTIST inverter circuit was done using the General Genelet Model described by Schaffter et al (Nat. Chem. 14, 1224-1232 (2022)). For all simulations, 25 nM of G1S1, and 250 nM of S1 were considered. Repressor production rate constant due to T7 RNAP transcription (kₚᵣ) was set at 0.002 sec⁻¹, and the RNA degradation rate constant due to (k_{dH}) was set at 0.0001 sec⁻¹. Concentrations of the dART_{IFN}-R1 were considered based on steady-state transcription results using T7 RNAP and RNase H, and are as follows:
dART_{IFNγ}-R1 with 0 nM of IFN-γ: 25 nM
dART_{IFNγ}-R1 with 25 nM of IFN-γ: 2.5 nM
dART_{IFNγ}-R1 with 250 nM of IFN-γ: 2 nM
dART_{IFNγ}-R1 with 2500 nM of IFN-γ: 0.1 nM.

Comparator circuit simulation variables Simulation for the ARTIST comparator circuit was done using the ctRSD Model by Schaffter et al (Sci. Adv. 8, eab14354 (2022)). For all simulations, 25 nM of Ref template, and 250 nM of Rep2b reporter were considered. Transcription rate constants for dART and Ref templates were (kₜₓₙ) were set at 0.002 sec⁻¹, 0.004 sec⁻¹, and 0.006 sec⁻¹ for the different T7 RNAP concentrations of 2, 4 and 8 U/μL, respectively. To simulate transcription profiles for 0, 5, 10, 20, 50, 75, and 100 nM of IFN-γ, dART concentrations of 50, 49, 48, 40, 16, 14, and 12 nM, respectively, were used.

Code Availability Simulations for K_{d,apparent}, reacted reporter kinetics of dARTs, reacted reporter kinetics and dose-response curves of the comparator are available at: github.com/hlee260/ARTIST.

TABLE 1

| dART non-template | Sequence | SEQ ID NO |
|---|---|---|
| dART-T7p-nt/ | TTCTAATACGACTCACTATAGGGATG | 1 |
| Prom-dART-nt | | |
| dART-Rep2b-nt | CATCCCCTACATCCACATACTAATTAACCTTC | 2 |
| dART-Repla-nt/ | CATCCCCTACTTTCACTTCACAACATCACTTC | 3 |
| O2-dART-nt | | |
| dART-RO1-nt | CATCCCGACATACAGATTAACCAGACAGTGACCTTC | 4 |
| O1-dART-nt | CATCCCCTACATCCACATACTAATTAAC | 5 |
| O3-dART-nt | CATCCCTACCATCACATTCAATAATCCT | 6 |
| dART template | Sequence | |
| dART-Rep2b-IFN-t | GAAGGTTAATTAGTATGTGGATGTAGGGGATGTGGGGTTGG | 7 |
| | TTGTGTTGGGTGTTGTGTCATCCCTATAGTGAGTCGTATTA | |
| dART-Rep2b-Thr-t | GAAGGTTAATTAGTATGTGGATGTAGGGGATGAGTCCGTGGT | 8 |
| | AGGGCAGGTTGGGGTGACTCATCCCTATAGTGAGTCGTATT | |
| | A | |
| dART-Rep2b-IL6-t | GAAGGTTAATTAGTATGTGGATGTAGGGGATGTGGTGGCAG | 9 |
| | GAGGACTATTTATTTGCTTTTCTCATCCCTATAGTGAGTCGT | |
| | ATTA | |
| dART-Rep2b-TNF-t | GAAGGTTAATTAGTATGTGGATGTAGGGGATGTGGTGGATG | 10 |
| | GCGCAGTCGGCGACAACATCCC TATAGTGAGTCGTATTA | |
| IFN-O1-dART-t | GTTAATTAGTATGTGGATGTAGGGGATGTGTTAGGGGGTTG | 11 |
| | GTTTTGGGTTGGCATCCC TATAGTGAGTCGTATTAGAA | |
| Dummy-O1-dART-t | GTTAATTAGTATGTGGATGTAGGGGATGTTCTTCTTCTTCTT | 12 |
| | CTTCTTCTTCCATCCC TATAGTGAGTCGTATTAGAA | |
| Thr-O1-dART-t | GTTAATTAGTATGTGGATGTAGGGGATGAGTCCGTGGTAGG | 13 |
| | GCAGGTTGGGGTGACTCATCCCTATAGTGAGTCGTATTAGA | |
| | A | |
| IL6-O1-dART-t | GTTAATTAGTATGTGGATGTAGGGGATGTGGTGGCAGGAGG | 14 |
| | ACTATTTATTTGCTTTTCTCATCCCTATAGTGAGTCGTATTAG | |
| | AA | |
| TNF-O1-dART-t | GTTAATTAGTATGTGGATGTAGGGGATGTGGTGGATGGCGC | 15 |
| | AGTCGGCGACAACATCCC TATAGTGAGTCGTATTAGAA | |
| IFN-O2-dART-t | TGATGTTGTGAAGTGAAAGTAGGGGATGTGTTAGGGGGTTG | 16 |
| | GTTTTGGGTTGGCATCCC TATAGTGAGTCGTATTAGAA | |
| IFN-O3-dART-t | AGGATTATTGAATGTGATGGTAGGGATGTGTTAGGGGGTTG | 17 |
| | GTTTTGGGTTGGCATCCC TATAGTGAGTCGTATTAGAA | |
| KMy-O1-dART-t | GTTAATTAGTATGTGGATGTAGGGGATGTGGGGGTTGAGGC | 18 |
| | TAAGCCGA CATCCCTATAGTGAGTCGTATTAGAA | |
| VEGF-O1-dART-t | GTTAATTAGTATGTGGATGTAGGGGATGTGTGGGGGTGGAC | 19 |
| | TGGGTGGGTACC CATCCCTATAGTGAGTCGTATTAGAA | |
| RNA output | Sequence | |
| dART-Rep2b-IFN-r | GGGAUGACACAACACCCAACACAACCAACCCCACAUCCCCUA | 20 |
| | CAUCCACAUACUAAUUAACCUUC | |
| dART-Rep2b-Thr-r | GGGAUGAGUCACCCCAACCUGCCCUACCACGGACUCAUCCCC | 21 |
| | UACAUCCACAUACUAAUUAACCUUC | |
| dART-Rep2b-IL6-r | GGGAUGAGAAAAGCAAAUAAAUAGUCCUCCUGCCACCACAU | 22 |
| | CCCCUACAUCCACAUACUAAUUAACCUUC | |
| dART-Rep2b-TNF-r | GGGAUGUUGUCGCCGACUGCGCCAUCCACCACAUCCCCUAC | 23 |
| | AUCCACAUACUAAUUAACCUUC | |
| DNA Reporter | Sequence | |
| Rep2b-F/O1_f | /5HEX/CTACATCCACATACTA | 24 |
| Rep2b-Q/O1′_q | GTTAATTAGTATGTGGATGTAG/3IAbRQSp/ | 25 |
| S1-F/O4_f | /Cy3/GACATACAGATTAACCAGACA | 26 |
| S1-Q/O4′_q | GTCACTGTCTGGTTAATCTGTATGTC/3IAbRQ/ | 27 |
| O2_f | /56-FAM/CTACTTTCACTTCACAA | 28 |
| O2′_q | TGATGTTGTGAAGTGAAAGTAG/3IABKFQ/ | 29 |
| O3_f | /56-FAM/TACCATCACATTCAAT | 30 |
| O3′_q | AGGATTATTGAATGTGATGGTA/3IABKFQ/ | 31 |
| Inverter circuit | Sequence | |
| dART-R1-nt | CATCCCTGCACGCCAAACCGTGGCGACGTAATAGAGCTGGA | 32 |
| dART-IFN-R1-t | TCCAGCTCTATTACGTCGCCACGGTTTGGCGTGCAGGGATGTG | 33 |
| | GGGTTGGTTGTGTTGGGTGTTGTGTCATCCCTATAGTGAGT | |
| | CGTATTAGAA | |
| IFN-rR1 | GGGAUGACACAACACCCAACACAACCAACCCCACAUCCCUGC | 34 |
| | ACGCCAAACCGUGGCGACGUAAUAGAGCUGGA | |
| dART-R2-nt | CATCCCTTCAGGTCAATAAGTGACCAAGTAATAGTGGGATG | 35 |
| dART-IFN-R2-t | CATCCCACTATTACTTGGTCACTTATTGACCTGAAGGGATGTG | 36 |
| | GGGTTGGTTGTGTTGGGTGTTGTGTCATCCCTATAGTGAGT | |
| | CGTATTAGAA | |
| IFN-rR2 | GGGAUGACACAACACCCAACACAACCAACCCCACAUCCCUUC | 37 |
| | AGGUCAAUAAGUGACCAAGUAAUAGUGGGAUG | |
| dA1 | TCCAGTATTACGTCGCCACGGTTTGGCGTGCC | 38 |
| G1S1-nt | GGCACGCCAAACCGTGGCGACGTAATACGACTCACTATAGGG | 39 |
| | AGAAAATCTCCC GACATACAGATTAACCAGACAGTGAC | |
| S1-t | GTCACTGTCTGGTTAATCTGTATGTCGGGAGATTTTCTCCCTAT | 40 |
| | AGTGAGTCG | |
| rS1 | GGGAGAAAAUCUCCCGACAUACAGAUUAACCAGACAGUGAC | 41 |
| dB1/B1 | GAGTAGGTCGTCGCCACGGTTTGGCGTGCATGGAAGGA | 42 |
| dA2 | TCCAGTATTACGTCGCCACGGTTTGGCGTGCC | 43 |
| G2C1-nt | GGCACGCCAAACCGTGGCGACGTAATACGACTCACTATAGGG | 44 |
| | AGAAAATCTCCC GACATACAGATTAACCAGACAGTGAC | |
| C1-t | GTCACTGTCTGGTTAATCTGTATGTCGGGAGATTTTCTCCCTAT | 45 |
| | AGTGAGTCG | |
| rC1 | GGGAGAAAAUCUCCCGACAUACAGAUUAACCAGACAGUGAC | 46 |
| Comparator circuit | Sequence | |
| dART-Rep2bc-nt | TTCTAATACGACTCACTATAGGGATG | 47 |
| Ref-Rep2b-nt | CATCCCCTACATCCACATACTAATTAACCTTC | 48 |
| dART-IFN-Rep2bc-t | ATCCACATACTAATTAACGGGATGTGGGGTTGGTTGTGTTG | 49 |
| | GGTGTTGTGTCATCCCTATAGTGAGTCGTATTAGAA | |
| Ref-Rep2b-t | GTTAATTAGTATGTGGATGTAGGGGATGGACAAGGAAAATCC | 50 |
| | TTCAATGAAGTGGGTCCATCCCTATAGTGAGTCGTATTAGAA | |
| O1′-dART-nt | CATCCCGTTAATTAGTATGTGGAT | 51 |
| IFN-O1′-dART-t | ATCCACATACTAATTAACGGGATGTGTTAGGGGGTTGGTTT | 52 |
| | TGGGTTGGCATCCCTATAGTGAGTCGTATTAGAA | |
| Amplified | | |
| Comparator Circuit | | |
| Strands | Sequence | |
| C1-dART-nt | CATCCCTCCTTCCATGAACGCCAAACCGTGGCGACGACCTACT | 53 |
| | C | |
| Ref-C1-dART-t | GAGTAGGTCGTCGCCACGGTTTGGCGTTCATGGAAGGAGGGA | 54 |
| | TGAGTCCGTGGTAGGGCAGGTTGGGGTGACTCATCCCTAT | |
| | AGTGAGTCGTATTAGAA | |
| C1′-dART-nt | CATCCCAAAGAGTAGGTCGTTTCATGGAAGGA | 55 |
| IFN-C1′-dART-t | TCCTTCCATGAAACGACCTACTCTTTGGGATGTGTTAGGGGG | 56 |
| | TTGGTTTTGGGTTGGCATCCCTATAGTGAGTCGTATTAGAA | |
| G1O4-nt | TCCTTCCATGCACGCCAAACCGTGGCGACGTAATACGACTCAC | 57 |
| | TATAGGGAGATTCGTCTCCCGACATACAGATTAACCAGACAG | |
| | TGAC | |
| A1 | TCCAGCTCTATTACGTCGCCACGGTTTGGCGTGCA | 58 |
| O4-t | GTCACTGTCTGGTTAATCTGTATGTCGGGAGACGAATCTCCCT | 59 |
| | ATAGTGAGTCG | |
| O4-dART-nt | CATCCCGACATACAGATTAACCAGACAGTGAC | 60 |
| Ref-O4-dART-t | GTCACTGTCTGGTTAATCTGTATGTCGGGATGAGTCCGTGGT | 61 |
| | AGGGCAGGTTGGGGTGACTCATCCCTATAGTGAGTCGTATT | |
| | AGAA | |
| O4′-dART-nt | CATCCCGTCACTGTCTGGTTAATCTGTA | 62 |
| IFN-O4′-dART-t | TACAGATTAACCAGACAGTGACGGGATGTGTTAGGGGGTTG | 63 |
| | GTTTTGGGTTGGCATCCCTATAGTGAGTCGTATTAGAA | |

### Example 1

dART Design and Characterization

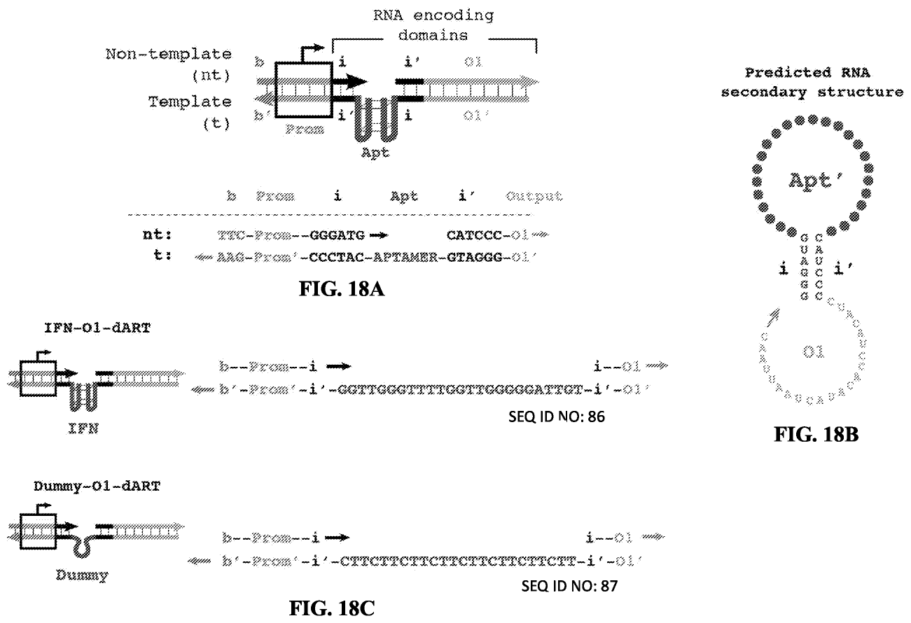

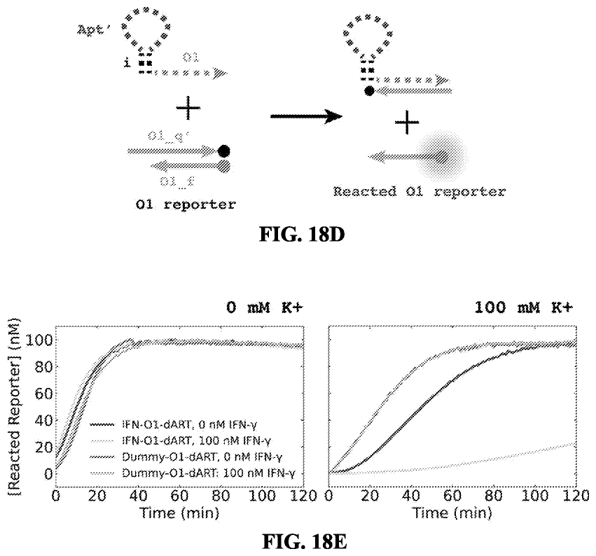

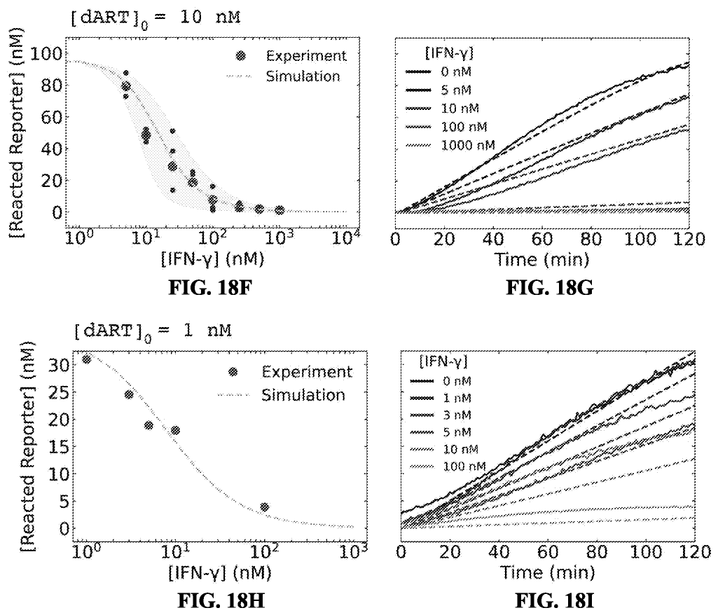

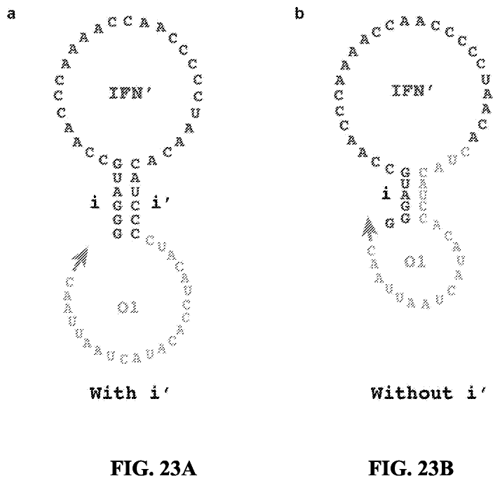

A general protein biosensing platform was developed by designing DNA transcription templates, termed dARTs, with G-quadruplex-forming aptamers downstream of a promoter that repress transcription via protein-aptamer binding. dARTs consist of a promoter domain that T7 RNA polymerase (T7 RNAP) can recognize to initiate transcription, a single-stranded aptamer domain that can bind to a specific protein, and an output domain that T7 RNAP transcribes to produce an RNA output sequence that can react downstream (FIG. 18A). The aptamer sequence was inserted into the template strand of the dART, e.g., the strand the polymerase reads during transcription. The output domain of the dART was designed to be double-stranded to prevent spurious interactions between the dART and other species such as the RNA transcribed from the dART. The aptamer and the output sequences are both transcribed from dARTs, so the resulting transcripts could have undesired secondary structure (FIG. 23). To reduce the possibility of such interactions, insulation domains, composed of i and i to the dART design were added (FIG. 18B). The i and i′ domains are complementary and are located on either side of the aptamer sequence so that in the RNA transcript produced from the dART, these domains will hybridize to form a hairpin, sequestering the sequence transcribed from the aptamer domain. (FIG. 18A). The i domain located just downstream of the promoter was designed to be 6 bases to facilitate efficient T7 RNAP transcription initiation.

For a protein to reduce the rate of dART transcription, the protein must be able to bind to the dART's aptamer and repress transcription, while under the same conditions dARTs should be efficiently transcribed when no protein is present. A dART was designed, termed the IFN-O1-dART, that contained an IFN-γ aptamer that forms a G-quadruplex to facilitate IFN-γ binding (FIG. 18C). For characterization, a fluorescently labeled DNA reporter complex was used. The DNA reporter complex reacts with IFN-O1-RNA via toehold-mediated strand displacement (TMSD) to displace one strand of the reporter complex to produce a fluorescent signal (FIGS. 18D and 24).

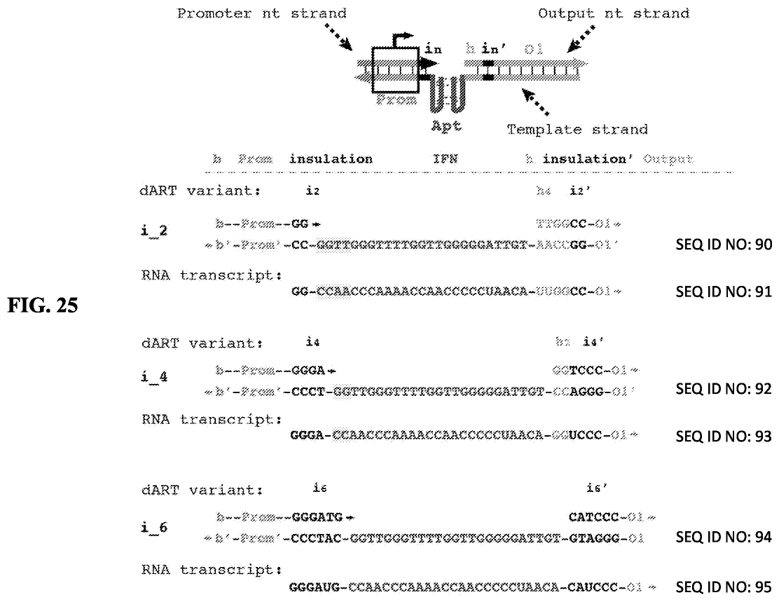

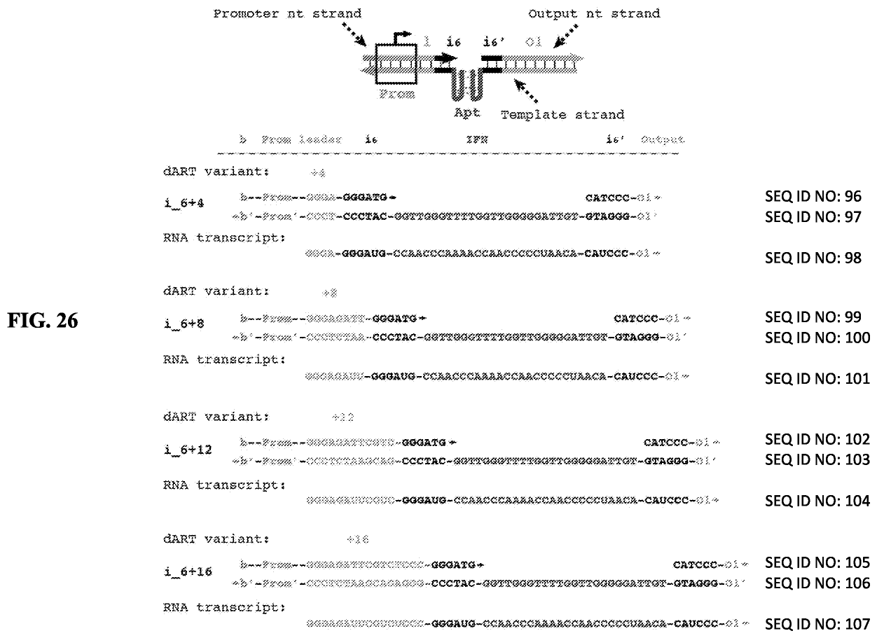

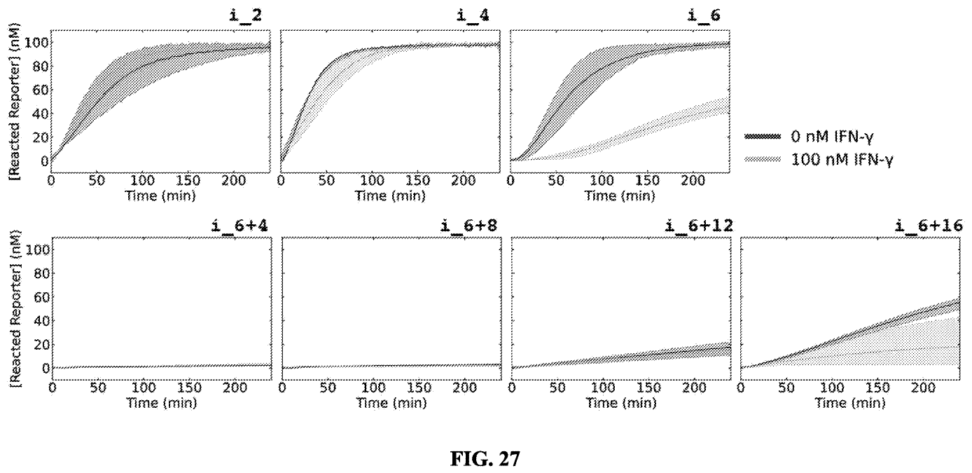

Many G-quadruplex aptamers require potassium ions for effective ligand binding. Consistent with this requirement, in the absence of K+, the same rate of fluorescence decrease (e.g., the rate of transcript reaction with the reporter) was observed for IFN-O1-dART in either the presence of 100 nM of IFN-γ or its absence (FIG. 18E), suggesting IFN-γ did not bind to the IFN-O1-dART to reduce transcription rate. When 100 mM KCl was also added, 100 nM IFN-γ was able to substantially repress transcription, consistent with IFN-γ binding to the dART. Without IFN-γ present, the rate of reporter reaction of the IFN-O1-dART was slightly lower with 100 mM of potassium than without potassium, which is consistent with the ability of G-quadruplexes to interrupt transcription. As a control, it was verified that a dART with a “dummy” aptamer domain (Dummy-O1-dART), e.g., no specific affinity for IFN-γ and no G-quadruplex forming domain, had similar reacted reporter kinetics for 0 nM or 100 nM of IFN-γ at both 0 mM and 100 mM of K+(FIGS. 18C and 18E). dART variants with i domain lengths ranging from 2 bases to 22 bases were also tested (FIGS. 25 and 26) and it was found that the 6 base design yielded the greatest difference in transcription rates with and without protein in our experimental conditions (FIG. 27).

A simple model was developed to predict the dose-response and kinetic behavior for 10 nM of IFN-O1-dART incubated with IFN-γ concentrations ranging between 0 nM and 1000 nM. The model assumes that transcription cannot occur when a protein is bound to a dART's aptamer, and thus the apparent dissociation constant of the protein and the dART's aptamer, which was termed K_{d,apparent}, will dictate the concentration of dART available for transcription at a given protein concentration (described below). This model qualitatively agrees with the measured dose-response curve for a K_{d,apparent} of 8 nM (FIGS. 18F and 28-30), which is on the same order as the measured K_{d} of the dART's aptamer sequence. Simulations of reacted reporter kinetics were also in good agreement with those measured in experiment (FIG. 18G). Using the model, the predicted dose-response curve using the K_{d,apparent} of 8 nM was compared to the experimental dose-response curve of 1 nM of IFN-O1-dART in the presence of (0, 1, 3, 5, 10, or 100) nM of IFN-γ. Simulated and experimental results were consistent with each other for both dose-response curve (FIG. 18H) and dose-dependent kinetic profiles (FIG. 18I).

dART Input and Output Modularity

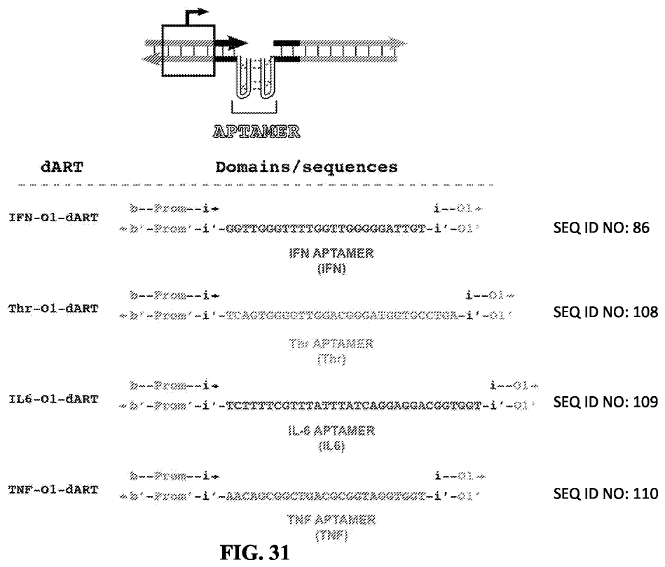

The IFN-γ aptamer in the IFN-O1-dART was replaced with aptamers for thrombin35, IL-636, and TNF-α37 to create tThr-O1-dART, IL6-O1-dART, and TNF-O1-dART (FIGS. 19A and 31). NUPACK predicted that the insulation domains would prevent undesired secondary structures from forming in these new output RNA sequences (FIG. 32).

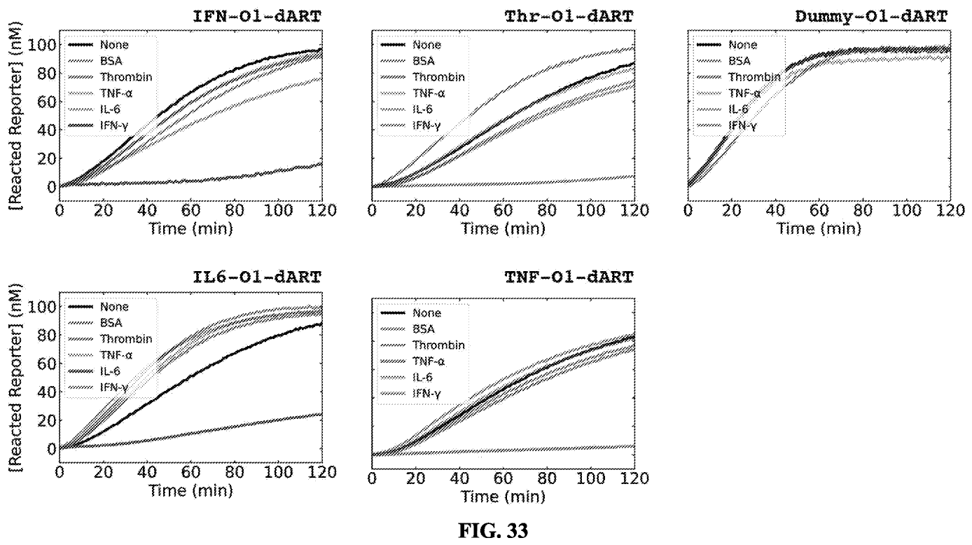

The reacted reporter kinetics for 10 nM of each of these dARTs were measured when combined with 0 nM to 1000 nM of their target proteins (FIG. 19B). For all dARTs, the rates of reacted reporter decreased with increasing protein concentrations. 10 nM of each dART and Dummy-O1-dART were combined with 100 nM of each protein ligand and BSA as a control. Only when dARTs were subjected to their target protein was there a large decrease in the reacted reporter concentration (FIGS. 19C and 33). As expected, the dummy template produced a similar concentration of reacted reporter in the presence of all input proteins.

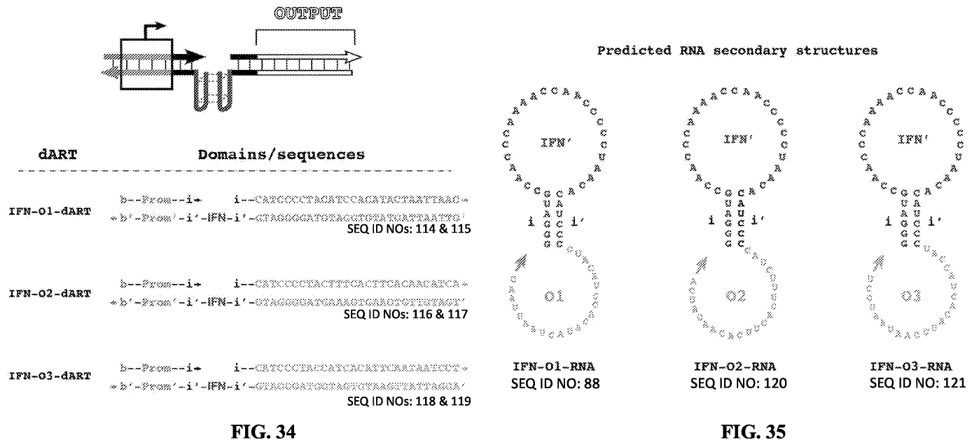

Next investigated was whether the output domain could also be exchanged modularly, which would allow dARTs to be easily connected to different downstream processes (FIG. 19D). Transcription profiles of IFN-O1-dART, IFN-O2-dART, and IFN-O3-dART which encode the O1, O2, and O3 output domains, respectively, were measured for 0 nM to 1000 nM of IFN-γ (FIGS. 34-35). dARTs with each output resulted in the expected reacted reporter dose-response curve across IFN-γ input concentrations (FIG. 19E), suggesting that the outputs of the dARTs could be easily customized to couple them to downstream circuits to create different types of biosensors.

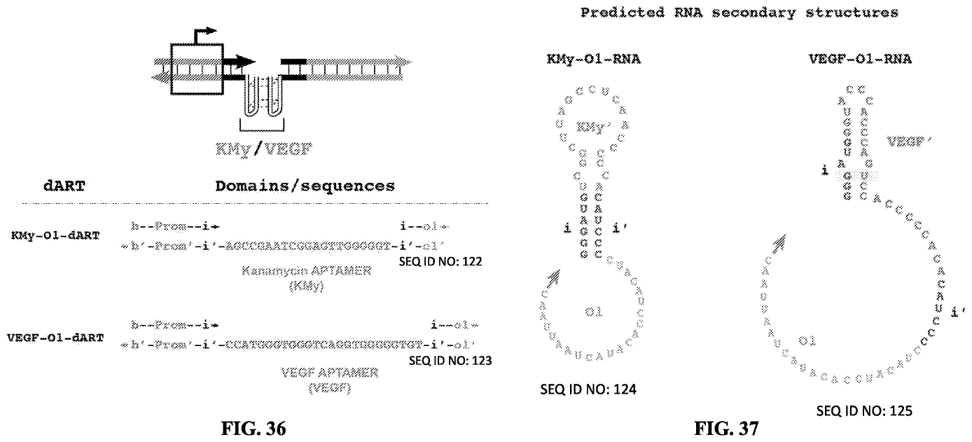

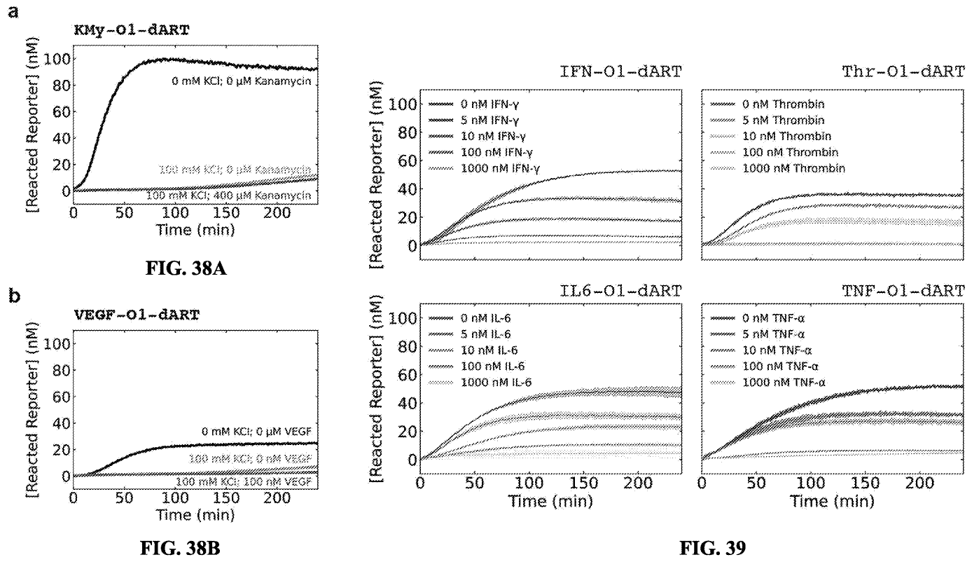

Not all aptamer domains integrated well with dARTs. For example, when aptamers for kanamycin and VEGF were introduced (FIG. 36), negligible reacted reporter was observed even in the absence of their corresponding ligands (FIG. 37). This could be because the aptamers form a stable enough G-quadruplex with K+ to repress transcription even without ligand, or because the RNA outputs form undesired secondary structure that impede their reaction with the reporter (FIG. 38).

### Example 2

### Analog Biosensors

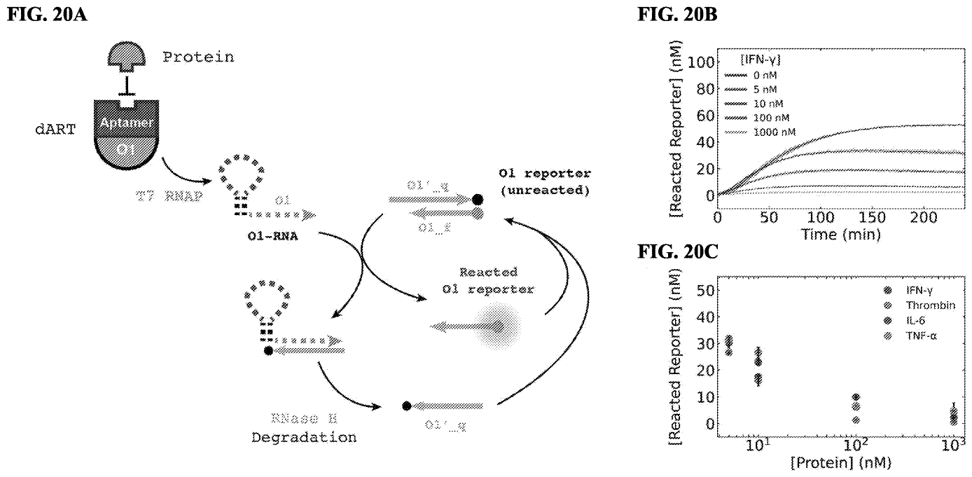

ARTIST can be used to build biosensors with different functionalities, such as analog or digital response, or signal amplification by coupling dARTs to downstream reactions. In principle, the rate of increase of reacted reporter by a dART is a measure of the target protein's concentration. However, rates are difficult to measure because they require observing a signal over time. Analog biosensors that indicate protein concentration as the steady-state concentration, of reacted reporter by adding RNase H, were built. RNase H degrades RNA outputs of a dART bound to the DNA strand O1′_q⁴⁰, allowing O1′_q to rehybridize to O1_f to reform the quenched reporter complex after degradation (FIG. 20A). A balance of RNA production and degradation should therefore produce a steady-state response whose magnitude is dependent on the concentration of the target protein. The reacted reporter concentrations of IFN-O1-dART with 0 nM to 1000 nM of IFN-γ indeed reached different steady-state values for different input protein concentrations in the presence of RNase H (FIGS. 20B and 20C). When Thr-O1-dART, IL6-O1-dART, and TNF-O1-dART were combined with reporter and 5 nM to 1000 nM of their corresponding input proteins, distinct steady-state reacted reporter concentrations were also observed (FIGS. 20C and 39).

### Digital Biosensors

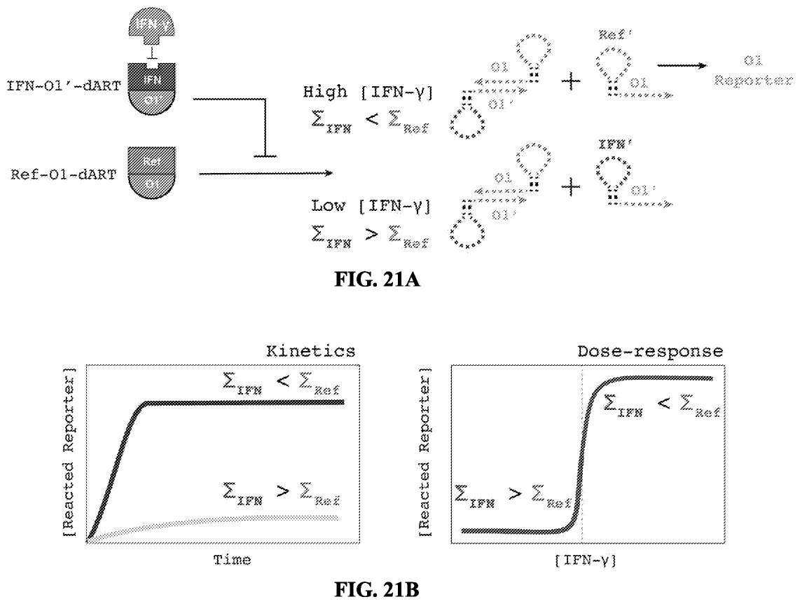

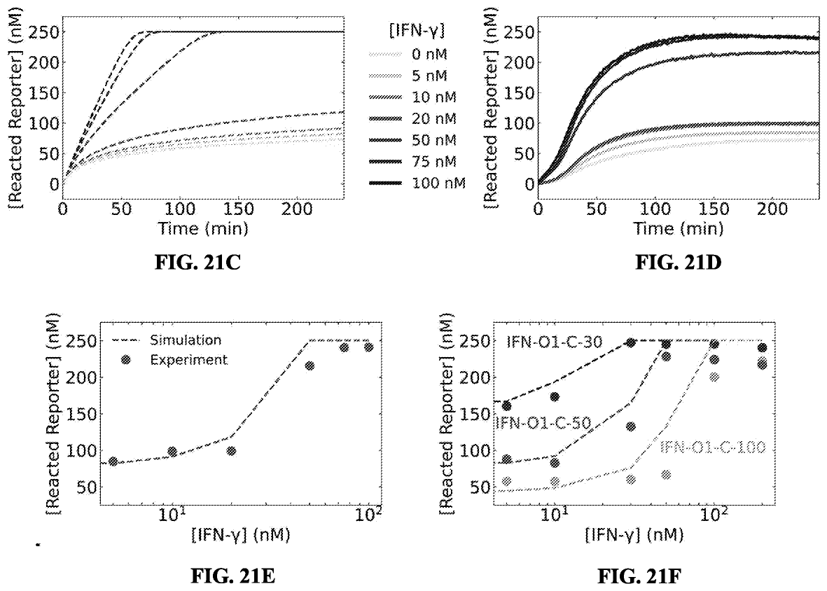

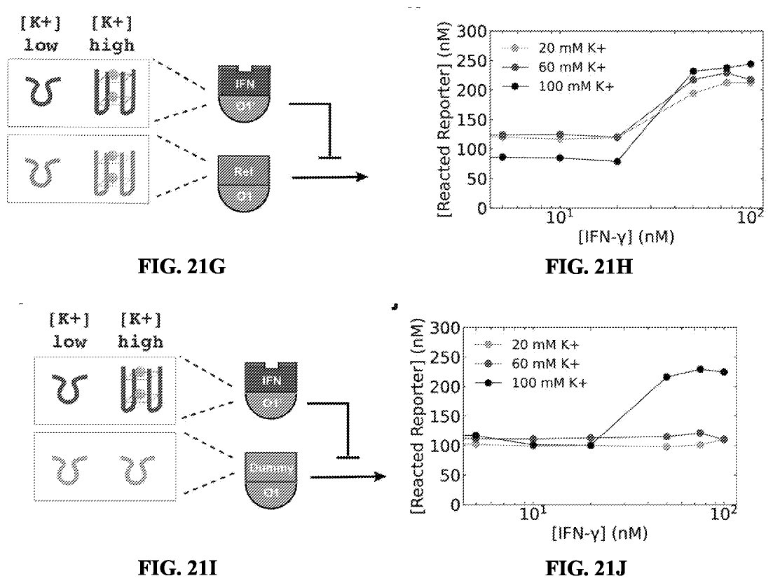

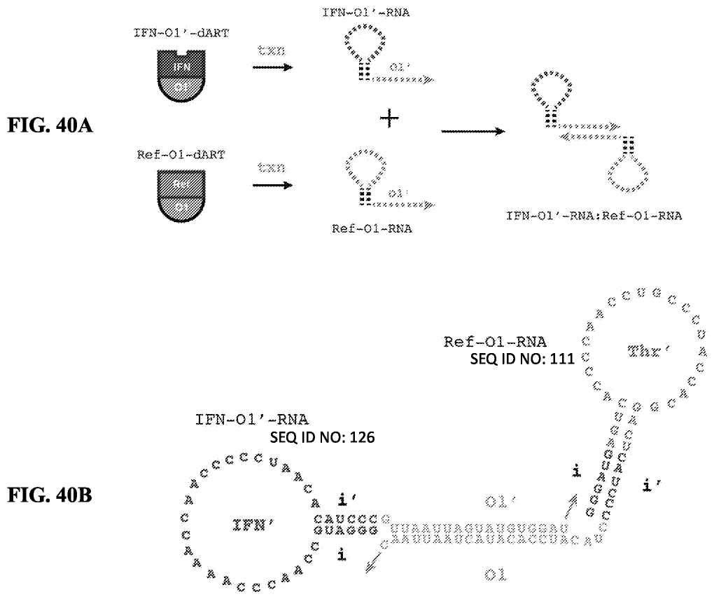

Digital biosensors are important for measuring whether a sample satisfies a specific diagnostic criterion. A digital biosensor was designed for IFN-γ by integrating two dARTs to produce a comparator circuit. A reference dART with an aptamer domain that does not bind IFN-γ was designed to produce the O1 output (Ref-O1-dART). Another dART with the IFN-γ aptamer domain was designed to produce an output with partial complementarity to the O1 sequence (IFN-O1′-dART) (FIGS. 21A and 40). If the IFN-O1′-dART is added at a higher concentration than the Ref-O1-dART, then IFN-O1′-RNA will sequester Ref-O1-RNA, resulting in a low reporter signal in the absence of protein. As IFN-γ concentration is increased, the rate of IFN-O1′-RNA transcription will decrease, allowing the Ref-O1-RNA to react with the O1 reporter once a threshold IFN-γ concentration is reached (FIG. 21B). This comparator circuit also inverts the output signal of individual dARTs, such that high protein concentration yields high output signal.

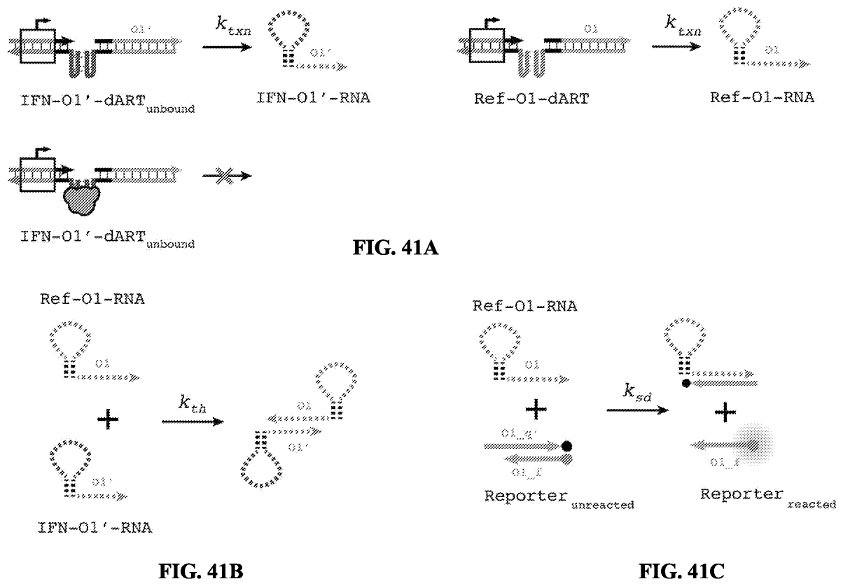

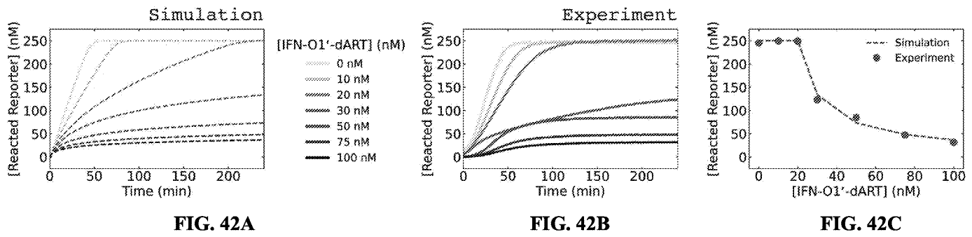

A comparator circuit could threshold the concentration of IFN-γ so that [IFN-γ]<20 nM would produce a low reacted reporter signal (OFF) and [IFN-γ]≥50 nM would produce a high signal (ON). Using simulations with the previously measured K_{d,apparent} for the IFN-O1-dART (FIG. 41) to determine that 25 nM of Ref-O1-dART and 50 nM of IFN-O1′-dART should produce a digital response with the desired threshold (FIGS. 21C and 42). These predictions were then confirmed in experiments (FIGS. 21D and 21E). This IFN-γ O1 comparator circuit involving 25 nM of Ref-O1-dART and 50 nM of IFN-O1′-dART, was referred to as IFN-O1-C-50.

The IFN-γ threshold of IFN-O1-C-50 could be tuned by changing the concentration of Ref-O1-dART, which would change the amount of IFN-O1′-dART that must be repressed by input protein to produce a reporter signal. An IFN-γ O1 comparator circuit with 15 nM of Ref-O1-dART required ≥100 nM IFN-γ to turn on (IFN-O1-C-100), while a circuit with 40 nM Ref-O1-dART only required ≥30 nM IFN-γ to turn on (IFN-O1-C-30; FIG. 21F). In principle, these circuits could measure the activation of CD4 T-cells, which can secrete above 30 nM of IFN-γ within microwells after 15 hours of mitogenic stimulation.

### Robust Digital Responses.

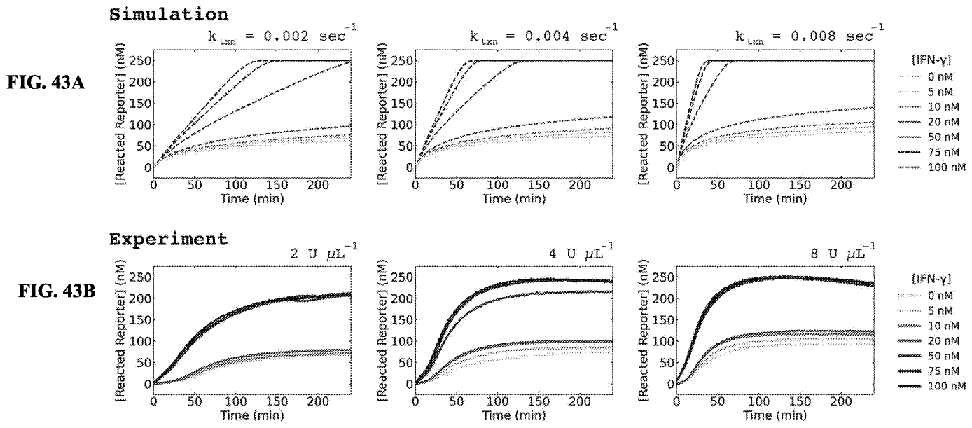

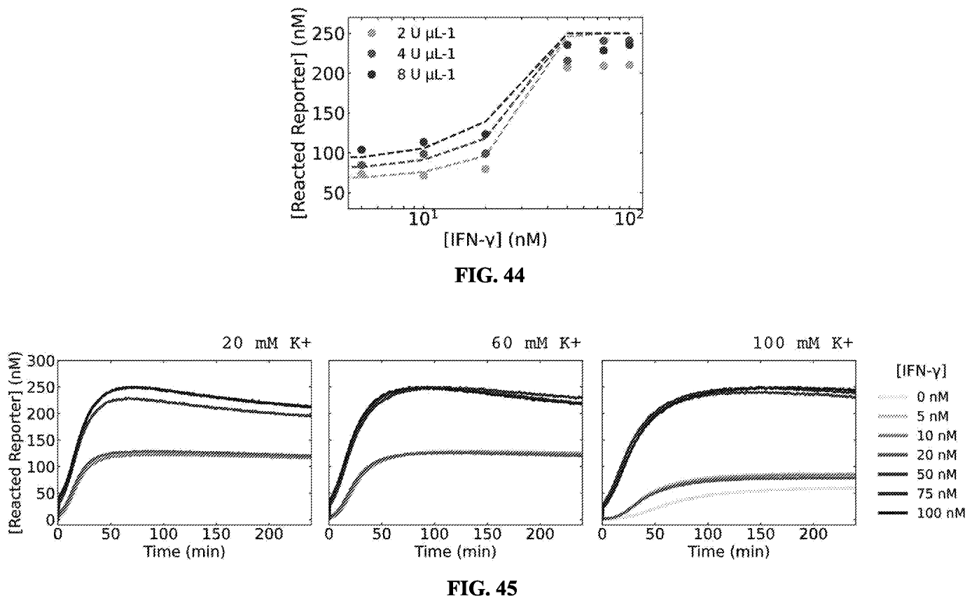

Digital response of IFN-O1-C-50 may also be insensitive to different environmental factors that often affect biosensors. For example, T7 RNAP activity can vary with solution composition or temperature. Since a comparator computes the ratio of transcription rates, the digital output should not change substantially with T7 RNAP activity, which would change both transcription rates. Indeed, the IFN-O1-C-50 with T7 RNAP activities of 2 U μl-1, 4 U μl-1, and 8 U μl-1 all produced low reacted reporter concentrations for [IFN-γ]<20 nM and high reacted reporter concentrations for [IFN-γ]≥50 nM (FIGS. 43-44).

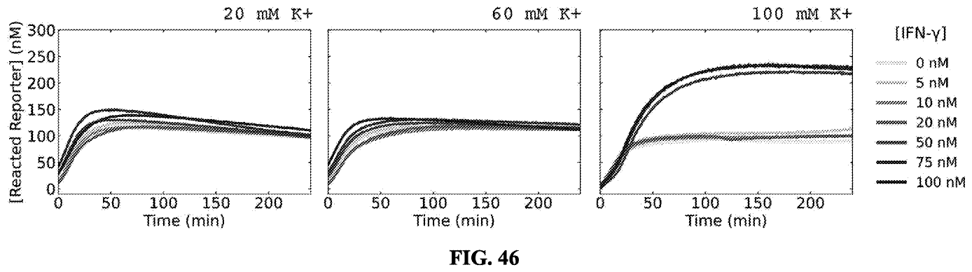

The affinity of G-quadruplex-forming aptamers for their ligands is also sensitive to potassium ion concentrations, and it was found that dART transcription rate decreased with increasing potassium concentration (FIG. 45). Despite the potassium sensitivity of individual dARTs, both dARTs comprising IFN-O1-C-50 contain G-quadraplexes (FIG. 21G), so their transcription rates are affected in similar ways by different potassium concentrations, resulting in similar digital responses and response thresholds for IFN-O1-C-50 for different potassium concentrations (FIG. 21H). In contrast, a comparator with a reference dART with a dummy aptamer sequence (FIG. 18C, Table 1) that does not form a G-quadruplex (FIG. 21I), becomes non-responsive at lower potassium ions concentrations (FIGS. 21J and 46) because the IFN-O1′-dART transcription rate changes with potassium concentration, but the Dummy-O1-dART transcription rate does not, changing the threshold. These results show how ARTIST can be used to construct digital biosensors that maintain their desired response profiles under different environmental conditions, which may eventually make it possible to construct sensors useful for different biological samples such as blood, urine, or saliva.

### Output Amplification

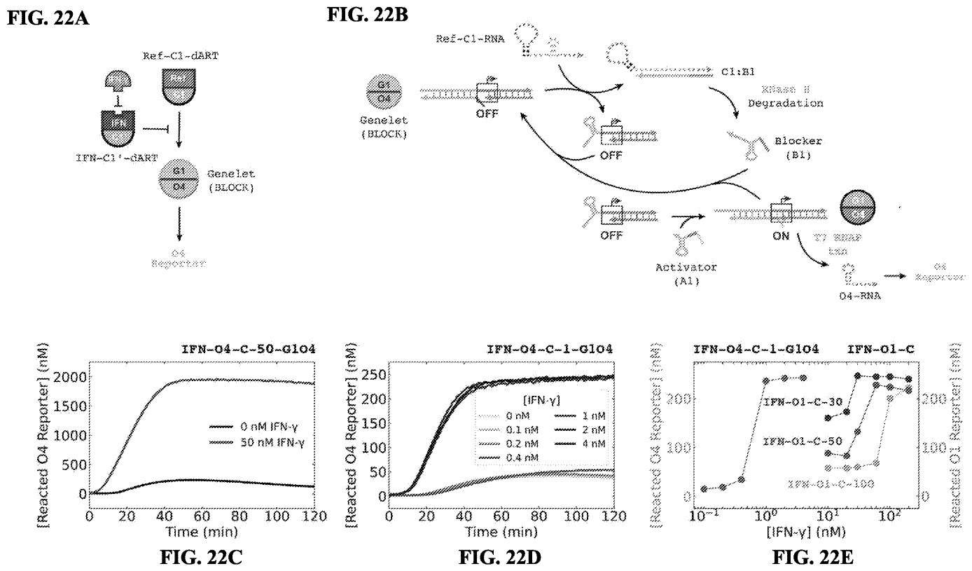

An amplified comparator (FIG. 22A) in which the RNA output of the comparator circuit activates transcription of a downstream genelet via strand displacement reactions that complete the promoter sequence of the genelet was designed (FIGS. 22B and 47-48). To prevent the low level of comparator output that is produced even when [IFN-γ] is low from triggering the output RNase H was included, such that only when the rate of Ref-C1-RNA production exceeds its degradation rate can it activate the genelet to produce an output.

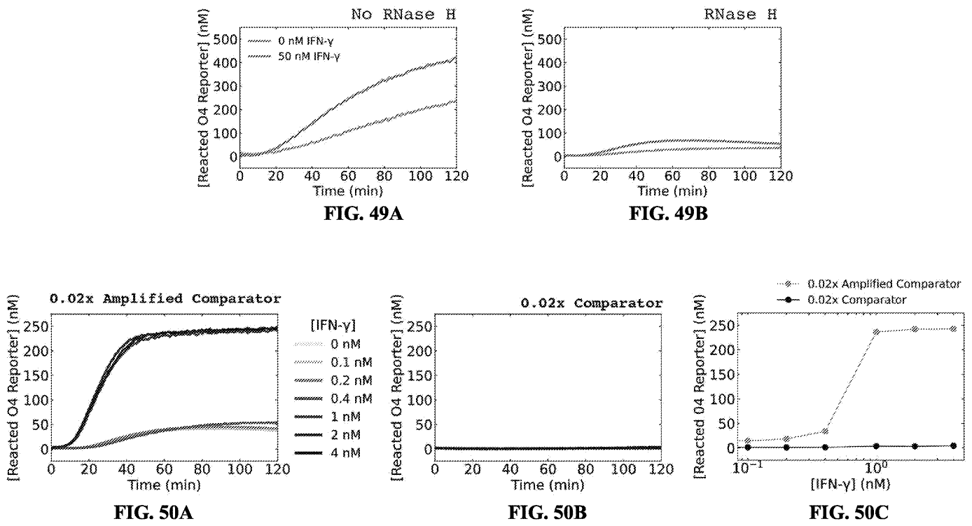

The amplified comparator (IFN-O4-C-50-G1O4) reacted with 2000 nM of O4 Reporter (FIG. 22C) in response to 50 nM of IFN-γ in <45 min, more than 40 times the output produced by the non-amplified comparator to the same input (IFN-O4-C-50; FIG. 49). Without IFN-γ, about 200 nM of O4 Reporter reacted transiently, but this amount then decreased, presumably because of RNase H-catalyzed O4 RNA degradation. Interestingly, the ON/OFF ratio of IFN-O4-C-50 was only 2-fold to 3-fold (FIG. 49), compared to IFN-O4-C-50-G1O4, which was nearly 10-fold (FIG. 22C). This increase in the fold-change between the ON and OFF signals may be due to two factors. First, Ref-O1-RNA reacts faster with the O1 reporter in IFN-O4-C-50 than Ref-C1-RNA does with B1 in IFN-O4-C-50-G1O4. Thus, when IFN-C1′-RNA is in excess, a greater fraction of Ref-C1-RNA is sequestered in IFN-O4-C-50-G1O4 than IFN-O4-RNA in IFN-O4-C-50. Second, in IFN-O4-C-50-G1O4, both Ref-C1-RNA and O4-RNA are degraded by RNase H, which results in a lower background signal without protein for IFN-O4-C-50-G1O4 than for IFN-O4-C-50.

An amplified comparator such as IFN-O4-C-50-G1O4 could be tuned to produce a similar amount of high output to its corresponding non-amplified comparator (IFN-O4-C-50) beginning above a much lower threshold protein concentration. To build this more sensitive amplified comparator, the concentrations of the Ref-C1-dART and IFN-C1′-dART of IFN-O4-C-50-G1O4 was reduced 50-fold to create a diluted amplified comparator, termed IFN-O4-C-1-G1O4. IFN-O4-C-1-G1O4 produced a high output for IFN-γ concentrations of 1 nM or higher and otherwise produced little output. Amplifying the output of the comparator thus made it possible to reduce the threshold input concentration of the circuit 50-fold (FIG. 50), while producing a similar amount of output when ON (FIGS. 22D-22E). IFN-O4-C-1-G1O4's 1 nM responsive threshold is lower than the apparent dissociation constant for the IFN-γ dART that we measured (8 nM). Furthermore, the amount of reacted reporter, a measure of the RNA output, produced by IFN-O4-C-1-G1O4 at this threshold is >250-fold higher than the input protein concentration (FIGS. 22D-22E).

### Example 3

### Overview of RNA Transcript Design

The i and i′ domains in the encoded transcripts of dARTs were designed so that they would hybridize, thereby creating a hairpin with a loop that sequesters the domain of the transcript whose sequence is complementary to the aptamer domain (apt′) inside. Such a loop should disfavor undesired RNA secondary structure formation or other interactions between the aptamer sequence of the dART and the RNA output sequence, ensuring that the output reacts reliably downstream. This design concept was tested using the IFN-O1 dART's RNA output. Without the i′ domain, the O1 domain of the transcript output may interact with the i domain and form undesirable secondary structures. With the i′ domain, NUPACK predicts that the only the i and i′ domains hybridize (FIG. 23).

To predict the secondary structures of the RNA transcripts in FIG. 23 and elsewhere herein, NUPACK 3.2.2 with a temperature of 37° C. and the default salt conditions (1 M Na⁺, 0 M Mg²⁺) was used. Although there is 6 mM MgCl₂ in the transcription buffer, there is a total of 8 mM of NTPs (2 mM of each ATP, UTP, CTP, GTP), which will sequester Mg²⁺, so the concentration of free Mg²⁺ is unknown. NUPACK cannot be used to predict G-quadruplex formation (as this is tertiary rather than secondary structure), so it was assumed that all single-stranded aptamer domains fold into a G-quadruplex structure due to 100 mM of K+ under ARTIST reaction conditions.

### Optimizing the Length of the Insulation Domain

For transcription factor proteins that repress transcription, the number of bases between the T7 promoter and the beginning of the repressor's operator sequence on the DNA template influences transcription and repression efficiency. It was hypothesized that the number of bases between the T7 promoter and the beginning of the aptamer domain on a dART could also influence aptamer-regulated transcription efficiency and the difference in unbound and protein-bound dART transcription rates. To explore this possibility, a series of dARTs were designed in which the length of the dsDNA domain between the promoter and aptamer domains was varied between 2 bases and 22 bases. All these dART variants were designed to produce a transcript with a 6 base hairpin to insulate the sequence transcribed from the aptamer domain from the output sequence. An h domain was introduced that was complementary to a portion of the apt′ sequence in the transcripts of the dART variants having fewer than 6 bases between the promoter and the aptamer domain (FIG. 25 and Table 2). For dARTs with more than 6 bases between the promoter and the aptamer domain, only the length of the sequence upstream of the insulation domain was extended, termed the leader, or 1, domain (FIG. 26 and Table 2).

The reacted reporter kinetics for the dART variants were characterized in the presence and absence of IFN-γ. dART variants with greater than 6 bases between the promoter and aptamer domain had slow reacted reporter kinetics even in the absence of IFN-γ. dART variants with 2 bases, 4 bases, or 6 bases between the promoter and aptamer domain had fast reacted reporter kinetics in the absence of IFN-γ, and only the 6 base variant had a substantial repression of reacted reporter signal with IFN-γ (FIG. 27). Based on these results, going forward all the dARTs were designed with 6 bases between the promoter and aptamer domains.

TABLE 2
DNA strands and sequences involved in optimizing the length between the
promoter and the aptamer domains of dARTs. The aptamer
domain is shown in bold. The i₂, i₄, i₆ domains are underlined
and designate lengths of 2 bases, 4 bases, and 6 bases, respectively
The sequences appended to i₆ (+4, +8, +12, +16) are the l
domains, which are shown in italics. The h domain, which is in italics
and underlined, is partially complementary to the aptamer
domain so that the encoded transcript of dARTs with (2 to 4)
base insulation domain lengths form a hairpin that prevents
the O1 domain from forming undesired secondary structures.
The portions of the aptamer sequence complementary to the h domain are both
bolded as well as shown in grey. dART variants with greater than
6 bases between the promoter and aptamer were prepared
using O1-dART-nt-i_6 for the output nt (non-template) strands.

| Promoter nt | Sequence | SEQ ID NO |
|---|---|---|
| Prom-dART-nt-i2 | TTCTAATACGACTCACTATA GG | 64 |
| Prom-dART-nt-i4 | TTCTAATACGACTCACTATA GGGA | 65 |
| Prom-dART-nt-i6 | TTCTAATACGACTCACTATA GGGATG | 66 |
| Prom-dART-nt-i6 + 4 | TTCTAATACGACTCACTATA GGGA GGGATG | 67 |
| Prom-dART-nt-i6 + 8 | TTCTAATACGACTCACTATA GGGAGATT GGGATG | 68 |
| Prom-dART-nt-i6 + 12 | TTCTAATACGACTCACTATA GGGAGATTCGTC GGGATG | 69 |
| Prom-dART-nt-i6 + 16 | TTCTAATACGACTCACTATA GGGAGATTCGTCTCCC | 70 |
| | GGGATG | |
| Output nt | Sequence | |
| O1-dART-nt-i_2 | TTGG CC TACATCCACATACTAATTAAC | 71 |
| O1-dART-nt-i_4 | GG TCCC TACATCCACATACTAATTAAC | 72 |
| O1-dART-nt-i_6 | CATCCC TACATCCACATACTAATTAAC | 73 |
| dART template | Sequence | |
| IFN-O1-dART-t-i2 | 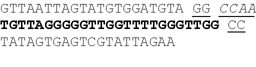 | 74 |
| IFN-O1-dART-t-i4 | 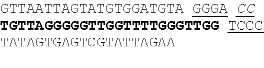 | 75 |
| IFN-O1-dART-t-i6 | GTTAATTAGTATGTGGATGTA GGGATG | 76 |
| | TGTTAGGGGGTTGGTTTTGGGTTGG CATCCC | |
| | TATAGTGAGTCGTATTAGAA | |
| IFN-O1-dART-t-i6 + 4 | GTTAATTAGTATGTGGATGTA GGGATG | 77 |
| | TGTTAGGGGGTTGGTTTTGGGTTGG CATCCC TCCC | |
| | TATAGTGAGTCGTATTAGAA | |
| IFN-O1-dART-t-i6 + 8 | GTTAATTAGTATGTGGATGTA GGGATG | 78 |
| | TGTTAGGGGGTTGGTTTTGGGTTGG CATCCC AATCTCCC | |
| | TATAGTGAGTCGTATTAGAA | |
| IFN-O1-dART-t-i6 + 12 | GTTAATTAGTATGTGGATGTA GGGATG | 79 |
| | TGTTAGGGGGTTGGTTTTGGGTTGG CATCCC | |
| | GACGAATCTCCC TATAGTGAGTCGTATTAGAA | |
| IFN-O1-dART-t-i6 + 16 | GTTAATTAGTATGTGGATGTA GGGATG | 80 |
| | TGTTAGGGGGTTGGTTTTGGGTTGG CATCCC | |
| | GGGAGACGAATCTCCC TATAGTGAGTCGTATTAGAA | |

### Example 4

Kinetic Modeling of dARTs

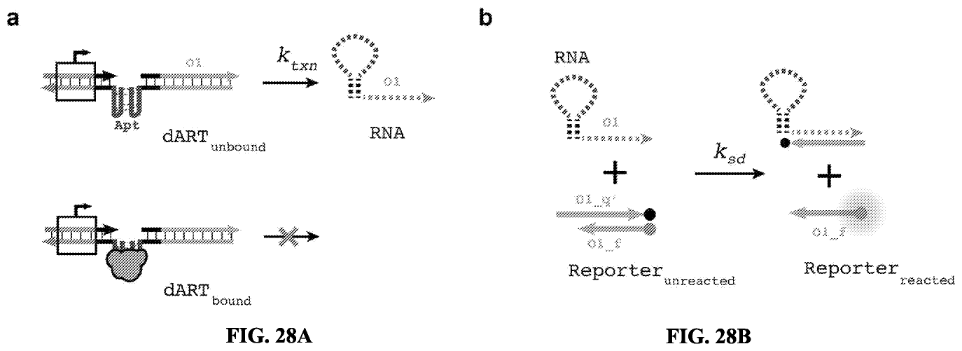

Modeling mass action rate equations A mass action kinetic model was used to predict changes in reacted reporter concentration over time. In the model, RNA is transcribed from an unbound dART and can bind to unreacted reporter (FIG. 28):

$$\frac{d [ R N A ]}{dt} = {{k}_{t x n} [ d A R T ]}_{u n b o u n d} - {{k}_{s d} [ R N A ] [ Reporter ]}_{u n r e a c t e d} , \qquad ( S1 )$$

$$\frac{{d [ Reporter ]}_{r e a c t e d}}{dt} = {{k}_{s d} [ R N A ] [ Reporter ]}_{u n r e a c t e d} . \qquad ( S2 )$$

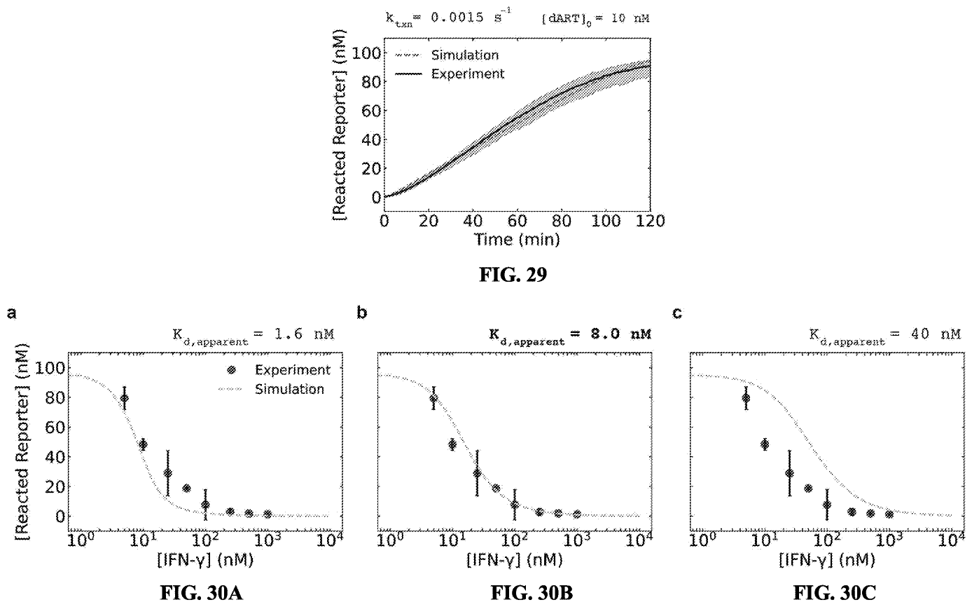

All simulations of Eqns S1 and S2 were conducted using Python's SciPy package (version 1.10.1) using the odeint library. In Eqns S1 and S2, [RNA] is the concentration of free RNA transcript, [dART]_{unbound} is the concentration of the unbound dART, [Reporter]_{unreacted} is the concentration of the unreacted DNA reporter, and [Reporter]_{reacted} is the concentration of reacted reporter. The rate of RNA production in the model is kₜₓₙ [dART]_{unbound}. kₜₓₙ is an apparent first order rate constant of transcription. [dART]_{unbound} is determined using Eqn S3 (see below). The strand displacement rate constant (k_{sd}) was assumed to be 1×10⁵ M⁻¹s⁻¹, which is consistent with previous measurements of rate constants for strand displacement with a toehold length of 6 bases. kₜₓₙ was fit as 0.0015 s⁻¹ using the experimentally measured reacted reporter kinetics when 10 nM IFN-O1-dART and 0 nM IFN-γ were present (FIG. 29).

The dARTs were incubated with their ligands for 30-60 min prior to adding T7 RNAP. In the model, it was assumed that dARTs and their ligands reach an equilibrium between bound and unbound dARTs that are determined by the apparent K_{d} between the protein and the aptamer and the protein concentration (as discussed below) during this initial incubation time.

It was also assumed that when a ligand is bound to the dART's aptamer's domain, the dART cannot be transcribed.

Modeling K_{d,apparent} and calculating unbound dART It was assumed that unbound dARTs can bind to their protein ligand with a binding affinity defined by its dissociation constant (K_{d}):

$${K}_{d} = \frac{{[ L ] [ d A R T ]}_{u n b ound}}{[ L : dART ]} , \qquad ( S3 )$$

where [L] and [L:dART] are the concentrations of free ligand and ligand-bound dART, respectively.

Based on the assumption that the binding reaction between the aptamer and the dART is at equilibrium, Eqn S3 was used to compute [dART]_{unbound} for a given initial dART concentration ([dART]₀) and initial ligand concentration ([L]₀) in terms of the dissociation constant. From mass balances [L]₀=[L]+[L:dART] and [dART]₀=[dART]_{unbound}+[L:dART], Eqn S3 can be expressed in terms of the initial concentrations L and dART and the bound dART concentration:

$${K}_{d} = \frac{( {[ L ]}_{0} - [ L : dART ] ) ( {[ d A R T ]}_{0} - [ L : dART ] )}{[ L : dART ]} . \qquad ( S4 )$$

Eqn S4 can be rearranged to solve for [L:dART] using the quadratic formula:

$$[ L : dART ] = \frac{\begin{matrix}{[ d A R T ]}_{0} + {[ L ]}_{0} + {K}_{d} - \\ \sqrt{{( {[ L ]}_{0} + {[ d A R T ]}_{0} + {K}_{d} )}^{2} - {{4 [ d A R T ]}_{0} [ L ]}_{0}}\end{matrix}}{2} \qquad ( S5 )$$

For the apparent K_{d} value for a given dART (K_{d,apparent}), Eqn S5 and [dART]_{unbound}=[dART]₀−[L:dART] can be used to predict the equilibrium concentrations of unbound IFN-O1-dART from [dART]₀ ([dART]₀ was 10 nM or 1 nM) for different initial concentrations of IFN-γ, i.e., [L]₀ (varied between 0 nM to 1000 nM).

K_{d,apparent} determination and validation Using Eqns S3, S4, and S5, K_{d}, was fit to the measured [Reporter]_{reacted} values after 120 min for 10 nM of IFN-O1-dART in the presence of (0, 5, 10, 25, 50, 100, 250, 500, or 1000) nM of IFN-γ (FIG. 18E, Table 3). This fit, termed the K_{d,apparent} value was 8 nM.

Kinetic simulations using the fit K_{d,apparent} exhibited good agreement with experimental measurements (FIG. 18F). For comparison, K_{d,apparent} values 5-fold higher or lower than the fit value of 8 nM did not produce a close correspondence with experimental results (FIG. 30). The K_{d,apparent} value of 8 nM was also consistent with experiments using a lower initial concentration of dART (FIGS. 18H and 18I).

TABLE 3
Predicted unbound IFN-O1-dART concentrations that correspond
to 0 nM to 1000 nM IFN-γ and rate constants for the
simulations presented in FIGS. 18F and 18G. The unbound IFN-O1-
dART concentration are calculated as described above using a
K_{d, apparent} value of 8 nM, an initial dART concentration
of 10 nM, and initial IFN-γ concentrations of (0,
5, 10, 25, 50, 100, 250, 500, or 1000) nM.

| [IFN-γ] | [IFN-O1-dART] | Rate | | |
|---|---|---|---|---|
| (nM) | (nM) | Parameters | Rate constants | |
| 0 | 10.0 | k_txn | 0.0015 | s⁻¹ |
| 5 | 7.6 | k_sd | 1 × 10⁵ | M⁻¹s⁻¹ |

| | |
|---|---|
| 10 | 5.8 |
| 25 | 3.1 |
| 50 | 1.6 |
| 100 | 0.8 |
| 250 | 0.3 |
| 500 | 0.2 |
| 1000 | 0.1 |

TABLE 4
Predicted unbound IFN-O1-dART concentrations that correspond
to 0 nM to 100 nM IFN-γ and rate constants for the simulations
presented in FIGS. 18H and 18I. Unbound IFN-O1-dART concentrations
were calculated as described above using a K_{d, apparent} value
of 8 nM, initial dART concentration of 10 nM, and initial IFN-
γ concentrations of (0, 1, 3, 5, 10, or 100) nM.

| [IFN-γ] | [IFN-O1-dART] | Rate | | |
|---|---|---|---|---|
| (nM) | (nM) | Parameters | Rate constants | |
| 0 | 1.0 | k_txn | 0.0046 | s⁻¹ |
| 1 | 0.9 | k_sd | 1 × 10⁵ | M⁻¹ s⁻¹ |

| | |
|---|---|
| 3 | 0.7 |
| 5 | 0.6 |
| 10 | 0.5 |
| 100 | 0.1 |

### Example 5

dART Modularity

Modular design of dART aptamer domains for different target proteins dART schematics with modular aptamer domains and their predicted RNA secondary structures are shown in FIGS. 31-32.

To verify the selectivity of the dARTs for their corresponding proteins, 10 nM of IFN-O1-dART, Thr-O1-dART, IL6-O1-dART, and TNF-O1-dART were incubated with 100 nM of either BSA, thrombin, TNF-α, IL-6, or IFN-γ in different samples, and measured the concentration of reacted reporter after 120 min of transcription for each sample (FIG. 33). Dummy-O1-dART was also combined with these sample proteins as a control. The endpoint reacted reporter concentrations in FIG. 33 were used to generate the heat map in FIG. 19C.

Different dART outputs dART schematics with modular output domains (O1, O2, O3) and their predicted RNA secondary structures are shown in FIGS. 34-35. Full sequences of the dART strands are provided in Table 1.

Aptamers that were Unsuccessfully Incorporated into dARTs

A KMy-O1-dART and a VEGF-O1-dART, which had aptamers for kanamycin and VEGF, respectively, were also designed (FIG. 36). These two aptamers were embedded into the general dART design, and NUPACK was used to predict the secondary structures of the RNA transcripts (FIG. 37).

10 nM of KMy-O1-dART and VEGF-O1-dART were mixed with 100 nM O1 DNA Reporter in the presence of either 0 mM or 100 mM potassium ions to see whether the dARTs would still transcribe when the formation of G-quadruplexes was favored (FIG. 38). KMy-O1-dART had fast reacted reporter kinetics in the absence of potassium, but with 100 mM KCl, the reacted reporter kinetics were minimal. This may be because the G-quadruplex structure of the kanamycin aptamer is stable enough to repress transcription even without a bound ligand. VEGF-O1-dART also showed relatively little reporter reaction regardless of 0 mM or 100 mM potassium. This could be due to unintended reactions of the RNA transcript because it is predicted to adopt a different structure than designed (FIG. 37).

### Example 6

### Digital Biosensor

Simulations of the comparator Simulations of the comparator were conducted using Python's SciPy package (version 1.10.1) using the odeint library. It was assumed that both Ref-O1-dART and IFN-O1′-dART without bound protein were transcribed by T7 RNAP with the same rate constant kₜₓₙ (FIG. 41A).

The rate constant of IFN-O1′-RNA binding to Ref-O1-RNA (kₜₕ) was assumed to be 1×10⁶ M⁻¹s⁻¹ (FIG. 41B). As described above, the strand displacement rate constant (k_{sd}) for the reaction between Ref-O1-RNA and [Reporter]_{unreacted} was assumed to be 1×10⁵ M⁻¹s⁻¹ (FIG. 41C). Eqns S6, S7, and S8 show the mass action rate equations that describe the dynamics of the comparator:

$$\frac{d [ Ref\_O1 \_RNA ]}{d t} = {k}_{t x n} [ Ref\_O1 \_dART ] - {k}_{t h} ( [ Ref\_O1 \_RNA ] [ {IFN\_O1}^{′} \_RNA ] ) - {{k}_{s d} [ Ref\_O1 \_RNA ] [ Reporter ]}_{u n r e a c t e d} \qquad ( S6 )$$

$$\frac{d [ {IFN\_O1}^{′} \_RNA ]}{d t} = {{k}_{t x n} [ {IFN\_O1}^{′} \_RNA ]}_{u n b o u n d} - {k}_{t h} ( [ Ref\_O1 \_RNA ] [ {IFN\_O1}^{′} \_RNA ] ) \qquad ( S7 )$$

$$\frac{{d [ Reporter ]}_{r e a c t e d}}{dt} = {{k}_{s d} [ Ref\_O1 \_RNA ] [ Reporter ]}_{u n r e a c t e d} . \qquad ( S8 )$$

The binding reaction between IFN-O1′-dART and IFN-γ was assumed to be at thermodynamic equilibrium prior to adding T7 RNAP, at which point the model applies. The concentration of unbound IFN-O1′-dART is determined as described above. The K_{d,apparent} of IFN-O1′-dART was assumed to be 8 nM, the value estimated above.

Determining which unbound IFN-O1′-dART concentrations produce high and low levels of comparator output To design the IFN-γ comparator, simulations were first used to predict the concentration of unbound IFN-O1′-dART above which little output would be produced from the comparator. To do so, the comparator with 25 nM of Ref-O1-dART and 250 nM of O1 Reporter and unbound IFN-O1′-dART concentrations ranging from 0 nM to 100 nM were simulated. It was assumed IFN-O1′-dART would be transcribed at the same rate as Ref-O1-dART.

These simulations predicted that the comparator should produce a relatively low concentration of reacted reporter when the concentration of unbound IFN-O1′-dART is ≥50 nM and should react with all of the reporter within 240 min when the concentration of unbound IFN-O1′-dART is ≤20 nM (FIG. 42A). These predictions were confirmed in experiments (FIGS. 42B-42C).

Determining the IFN-γ threshold for digital response of the comparator Having determined that 50 nM of IFN-O1′-dART would keep reporter signal low in the comparator, simulations were used to identify the concentration of IFN-γ that would repress transcription of IFN-O1′-dART enough to produce a digital ON signal. As described above, the concentration of unbound IFN-O1′-dART for IFN-γ concentrations of (0, 5, 10, 20, 50, 75, 100, or 200) nM and 50 nM of IFN-O1′-dART was calculated (Table 5). Kinetic simulations of the comparator were then used with these unbound IFN-O1′-dART concentrations to predict the comparator's response for the corresponding input protein concentrations (FIG. 21C). The simulations were also used to predict the behavior of the comparator for different concentrations of Ref-O1-dART (15 nM, 25 nM, 40 nM), which changes the threshold IFN-γ concentration (FIG. 21F).

TABLE 5
Predicted unbound dART concentrations were used to produce
the simulated reacted reporter kinetics in FIGS. 21C, 21e,
and 21F of the main text. The unbound IFN-O1′-dART concentration
for each input protein concentration was calculated as described
above with a K_{d, apparent} of 8 nM, a total IFN-O1′-dART
concentration of 50 nM, and IFN-γ concentrations of
(0, 5, 10, 20, 30, 50, 75, 100, or 200) nM.

| [IFN-γ] | [IFN-O1′-dART] | Rate | | |
|---|---|---|---|---|
| (nM) | (nM) | Parameters | Rate constants | |
| 0 | 50.0 | k_txn | 0.004 | s⁻¹ |
| 5 | 45.7 | k_th | 1 × 10⁶ | M⁻¹ s⁻¹ |
| 10 | 41.6 | k_sd | 1 × 10⁵ | M⁻¹ s⁻¹ |

| | |
|---|---|
| 20 | 33.8 |
| 30 | 26.9 |
| 50 | 16.4 |
| 75 | 9.4 |
| 100 | 6.2 |
| 200 | 2.5 |

Dose-response curves of the comparator are similar for different T7 RNAP concentrations If both IFN-O1′-dART and Ref-O1′-dART are transcribed at similar transcription rates, the dose response of the comparator should not vary significantly with changes in T7 RNAP activity. To test this hypothesis, the characterization of the IFN comparator in FIG. 21D was repeated using half or twice the T7 RNAP concentration. Similar dose-response curves were observed for 0 nM to 100 nM IFN-γ for the three T7 RNAP concentrations, although as predicted in simulations, the time to saturate the reporter signal decreased with increasing T7 RNAP concentration (FIGS. 43-44).

Insensitivity of the comparator to potassium ion concentration The reacted reporter kinetics corresponding to FIGS. 21H and 21J in the main text are in FIGS. 45-46, respectively.

### Example 7

### Amplified Digital Biosensor

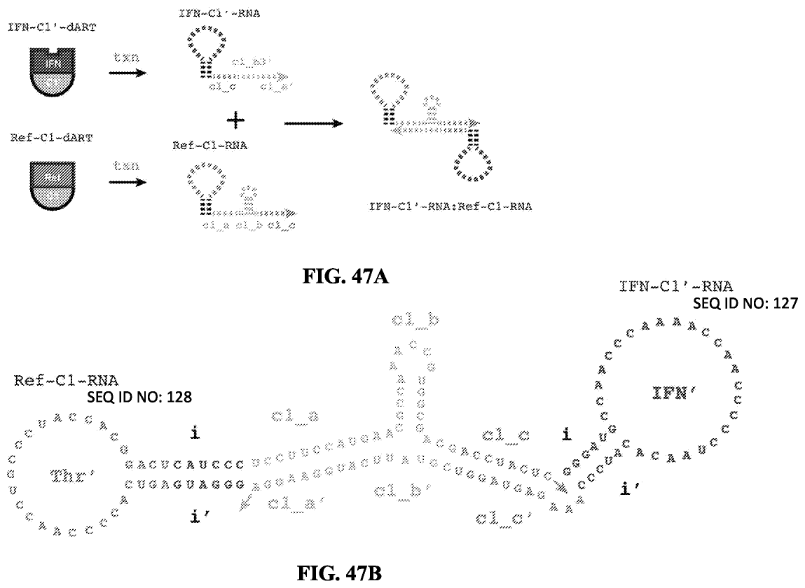

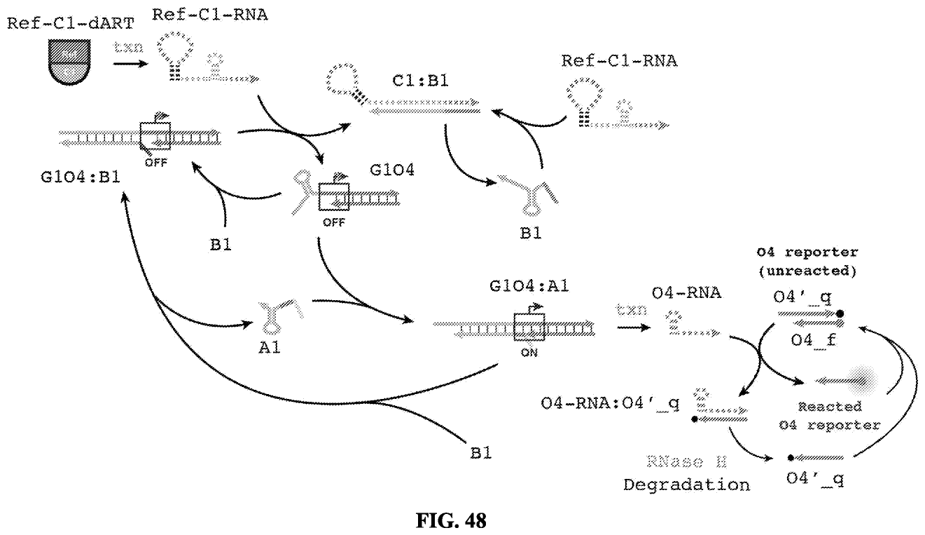

The amplified digital biosensor, or amplified comparator, consists of a comparator that compares the outputs of an IFN-γ dART and a reference dART, IFN-C1′-RNA and Ref-C1-RNA, and a blocked genelet that is activated for transcription by Ref-C1-RNA. NUPACK predicts that the C1 and C1′ domains of Ref-C1-RNA and IFN-C1′-RNA hybridize as designed (FIG. 47). The C1 domain also reacts with a genelet (FIG. 48), a DNA template whose transcription is regulated by RNA inputs via reactions developed previously. Specifically, C1 is the coactivator sequence for the genelet G1O4. G1O4 is in a blocked state because it is bound to the DNA blocker strand B1. The promoter sequence of the resulting complex, G1O4:B1, is incomplete, so it is not transcribed at significant rates. C1 can bind to B1, freeing G1O4. G1O4 can then bind to A1 to produce the complex G1O4:A1 which has a complete promoter. G1O4:A1 can be transcribed to produce O4 RNA that reacts with the reporter.

The amplified comparator also includes the enzyme RNaseH. Ref-C1-RNA bound to B1 can be degraded by RNase H to produce a free B1 strand. Free B1 can bind to G1O4 and can displace A1 from G1O4:A1. Both reactions return the genelet G1O4 to the blocked state G1O4:B1. Free B1 can also bind to intact Ref-C1-RNA. RNase H can also degrade O4-RNA bound to the O4′_q strand of the DNA Reporter, allowing O4′_q and O4_f to hybridize again to reduce fluorescence.

Note that the C1′ domain of IFN-C1′-RNA was designed not to be fully complementary to the hairpin in C1. This was done to prevent IFN-C1′-RNA from binding to G1O4, which would block the A1 from binding to G1O4 to activate it.

The 0.02× amplified comparator and 0.02× comparator use the same DNA strands as the amplified comparator and comparator, respectively, each at 0.02 times the concentrations of the amplified comparator and comparator. The responses of these two circuits to IFN-γ concentrations between 0 nM to 4 nM were compared. In its on state, the 0.02× amplified comparator produced up to 250-fold more RNA output than its protein input; the increase in the RNA output between the on state and the baseline state of the comparator was up to 200-fold the input protein concentration (FIG. 50A). In contrast, the 0.02× comparator produced negligible reacted O4 reporter for any concentration of IFN-γ between 0 nM to 4 nM (FIG. 50B).

### Example 8

### VEGF Aptamers

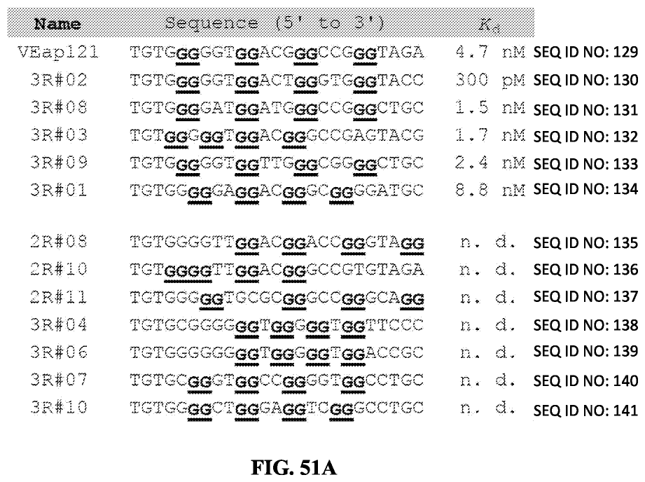

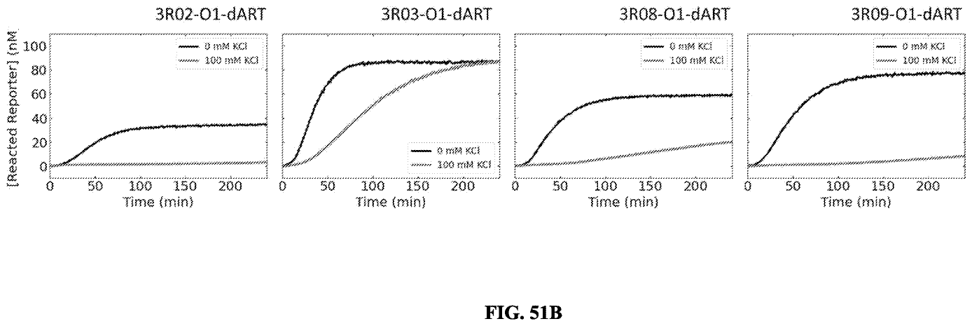

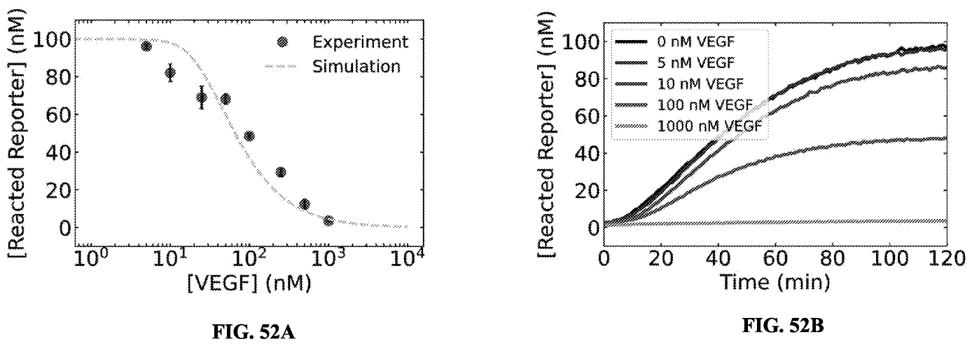

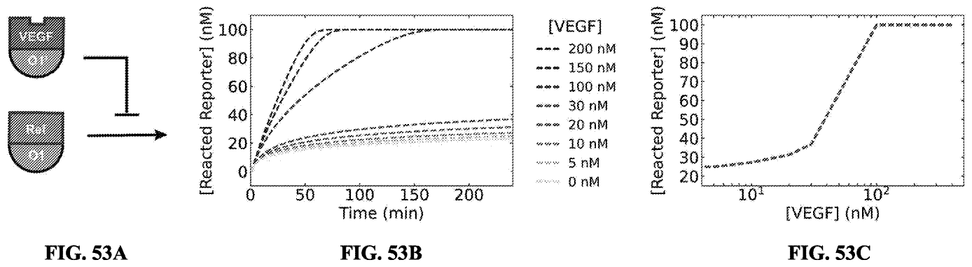

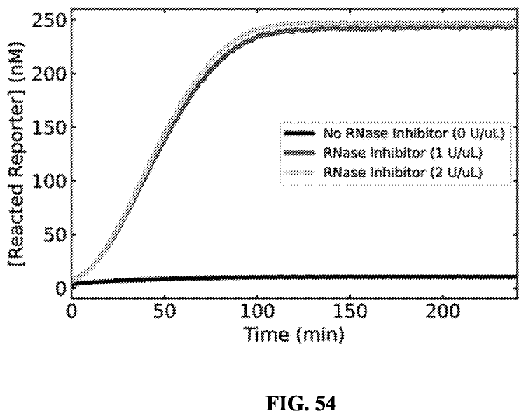

Tanaka et al. (Anal. Chem. 85, 1132-1137 (2013)), screened a variety of VEGF aptamers (FIG. 51A). Those aptamer sequences were used in various general dART designs and screened by measuring reporter kinetics with and without potassium ions (FIG. 51B). 3R02-O1-dART and 3R08-O1-dART appear to transcribe relatively poorly, whereas 3R03-O1-dART and 3R09-O1-dART are able to transcribe efficiently in the absence of potassium ions. The reduced kinetics of 3R03-O1-dART and 3R09-O1-dART with 100 mM KCl is consistent with our findings that the G-quadruplex formation represses transcription.

### Example 9

### ARTIST Reactions in Serum

Serum is known to have an abundance of nucleases (DNases and RNases) that can degrade nucleic acids, and hence the molecular biosensors described herein, over time. Actin, a known natural DNase inhibitor, and RNase inhibitors (e.g., N8080119; Applied Biosystems) can be used to facilitate biosensor operation in samples comprising serum (e.g., serum-supplemented cell culture environments).

### Example 10

### ARTIST in Hydrogels

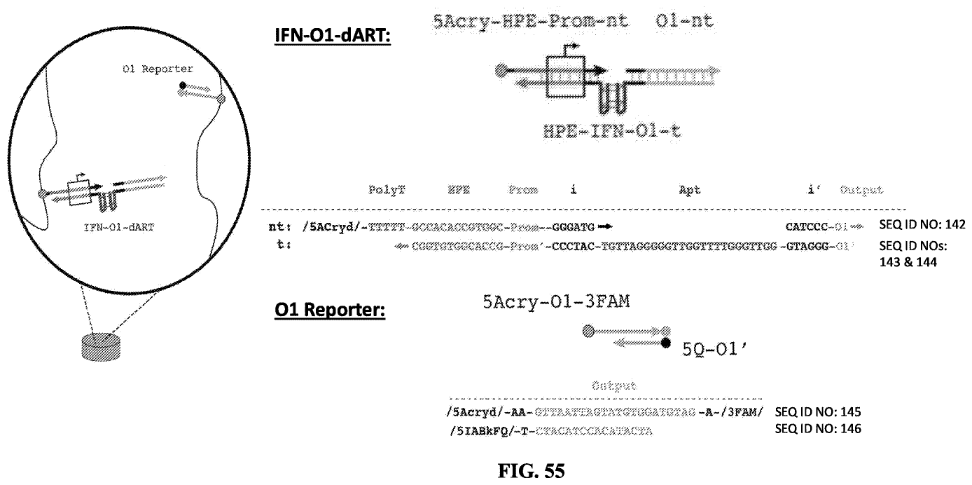

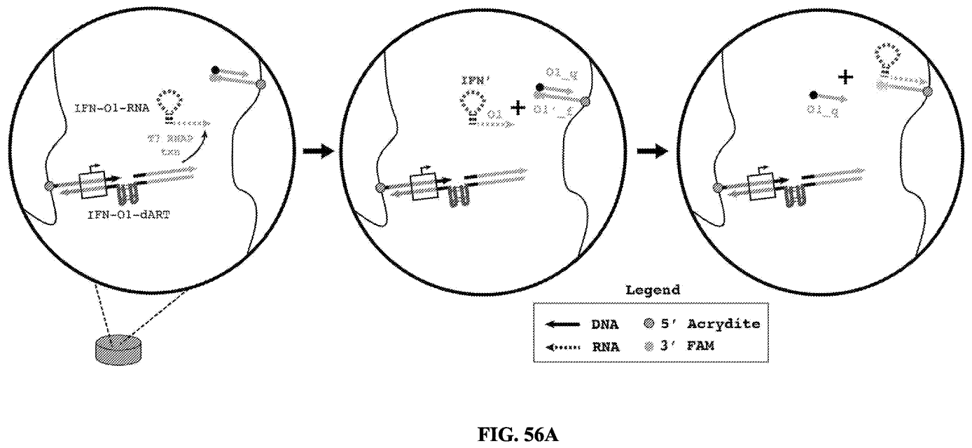

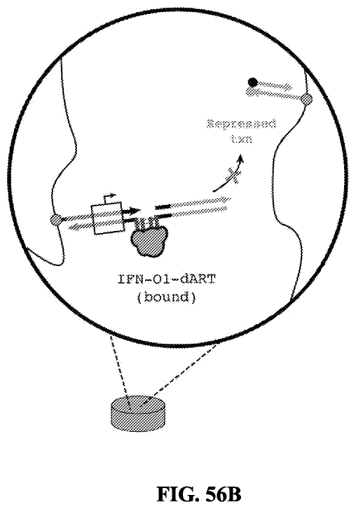

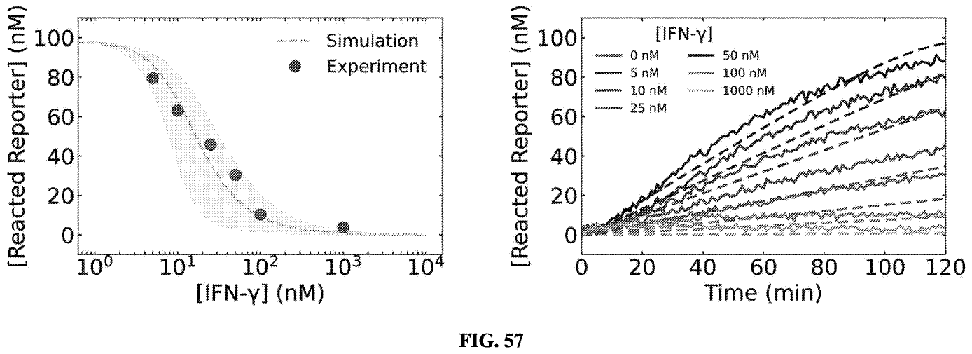

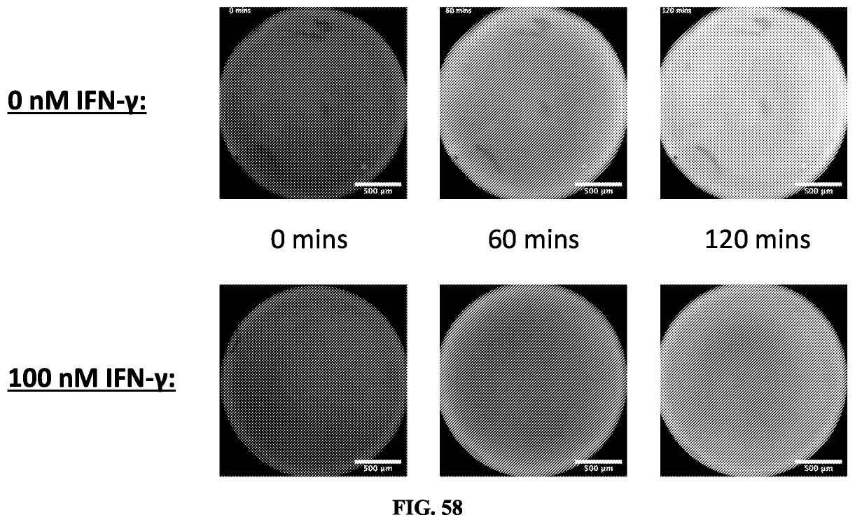

ARTIST can be coupled with hydrogels to create a protein-response hydrogel (FIG. 55). Hydrogels in which the dARTs are not bound to their corresponding protein exhibit substantial increase in fluorescent signal over time, whereas hydrogels in which dARTs are bound to their corresponding protein exhibit minimal fluorescent signal increase (FIGS. 56A-56B). The reacted reporter kinetics and the dose-response curve a hydrogel with dARTs demonstrated that this hydrogel system can detect IFN-γ (FIGS. 57 and 58). Simulations for the reacted reporter kinetics matched well with the experimental reacted reporter measurements. An apparent dissociation constant was determined based on experimental reacted reporter measurements.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A nucleic acid transcription template comprising an aptamer region configured to bind a molecule downstream of a promoter region and upstream of an output region which encodes an RNA output.

2. The nucleic acid transcription template of claim 1, wherein the aptamer region is flanked on each side by stem sequences.

3. The nucleic acid transcription template of claim 2, wherein the stem sequences are each 2-20 bases in length.

4. The nucleic acid transcription template of claim 2 or 3, wherein the stem sequences are each 5-7 bases in length.

5. The nucleic acid transcription template of any of claims 2-4, wherein the stem sequences are each 6 bases in length.

6. The nucleic acid transcription template of any of claims 1-5, wherein the nucleic acid transcription template is DNA.

7. The nucleic acid transcription template of any of claims 1-6, wherein the aptamer region is single stranded.

8. The nucleic acid transcription template of any of claims 1-7, wherein the promoter region and/or the output region is double stranded.

9. A system for the detection or measurement of at least one molecule comprising:
at least one first nucleic acid transcription template comprising an aptamer region configured to bind to the at least one molecule downstream of a promoter region and upstream of an output region which encodes at least one first RNA output; and
an RNA polymerase.

10. The system of claim 9, wherein the aptamer region is flanked on each side by stem sequences.

11. The system of claim 10, wherein the stem sequences are each 2-20 bases in length.

12. The system of claim 10 or 11, wherein the stem sequences are each 5-7 bases in length.

13. The system of any of claims 10-12, wherein the stem sequences are each 6 bases in length.

14. The system of any of claims 9-13, wherein the nucleic acid transcription template is DNA.

15. The system of any of claims 9-14, wherein the aptamer region is single stranded.

16. The system of any of claims 9-15, wherein the promoter region and/or the output region is double stranded.

17. The system of any of claims 9-16, wherein production of the at least one first RNA output is repressed in the presence of the at least one molecule.

18. The system of any of claims 9-17, further comprising one or more modules and/or components which utilize the at least one first RNA output as an input for signal processing, in vitro transcription networks and circuits, chemical reactions, or a combination thereof.

19. The system of any of claims 9-18, further comprising a nucleic acid reporter configured for detecting one or more of the at least one first RNA output.

20. The system of claim 19, wherein the nucleic acid reporter is double stranded comprising a first strand complementary to one or more of the at least one RNA output and a second strand with a detection moiety.

21. The system of claim 20, wherein the detection moiety comprises a radioisotope, mass isotope, dye, fluorophore, or hapten.

22. The system of any of claims 9-21, further comprising an RNA output inversion module.

23. The system of claim 22, wherein the RNA output inversion module comprises:
a second nucleic acid transcription template comprising at least a partially singled stranded promoter site and an output region encoding a second RNA output; and
a first nucleic acid activator strand which is complementary to the partially single stranded promoter site and the first RNA output.

24. The system of claim 23, wherein the second nucleic acid transcription template is configured to transcribe the second RNA output when the first nucleic acid activator strand is bound to the partially single stranded promoter.

25. The system of claim 24, wherein production of the second RNA output is increased in the presence of the molecule.

26. The system of any of claims 9-25, further comprising an RNA output amplification module.

27. The system of claim 26, wherein the RNA output amplification module comprises:
a third nucleic acid transcription template comprising a partially single stranded promoter site and a sequence encoding a third RNA output;
a second nucleic acid activator strand which is complementary to the partially single stranded promoter site on the third nucleic acid transcription template; and
a nucleic acid blocker strand complementary to the second RNA output and the single stranded promoter site on the third nucleic acid transcription template,
wherein the nucleic acid blocker strand is configured to prevent transcription from the third nucleic acid transcription template.

28. The system of any of claims 23-27, wherein the first nucleic acid activator strand, the second nucleic acid activator strand, and/or the nucleic acid blocker strand is DNA.

29. The system of any of claims 9-28, further comprising a self-calibration module comprising a reference nucleic acid transcription template lacking a single stranded aptamer domain and a sequence encoding a reference RNA output.

30. The system of claim 29, wherein the reference nucleic acid transcription template and the first nucleic acid transcription template are configured to transcribe at the same rate.

31. The system of claim 30, wherein the at least one first RNA output and the reference RNA output comprise complementary sequences.

32. The system of any of claims 29-31, wherein the self-calibration module comprises a reference reporter nucleic acid.

33. The system of claim 32, wherein the reference reporter nucleic acid is complementary to the reference RNA output.

34. The system of any of claims 9-33, wherein the system is embedded in a hydrogel.

35. The system of claim 34, wherein at least one of the nucleic acid transcription template and the nucleic acid reporter are linked to a matrix molecule of the hydrogel.

36. The system of claim 34 or 35, wherein at least one of: the second nucleic acid transcription template, first nucleic acid activator strand, third nucleic acid transcription template, second nucleic acid activator strand, nucleic acid blocker strand, reference nucleic acid transcription template are linked to a matrix molecule of the hydrogel.

37. The system of any of claims 9-36, wherein the system further comprises a ribonuclease.

38. The system of any of claims 9-37, wherein the system further comprises a sample.

39. The system of any of claims 9-38, wherein the sample is a biological sample.

40. The system of any of claims 9-39, wherein the molecule is a biomolecule.

41. The system of any of claims 9-40, wherein the molecule is a biomarker.

42. The system of any of claims 9-41, wherein the molecule is a protein.

43. The system of any of claims 9-42, further comprising an RNA detection component.

44. A method for detecting or measuring the level of at least one molecule comprising:
incubating a sample comprising the at least one molecule with a system of any one of claims 9-43; and
measuring the at least one first RNA output or a signal corresponding to the at least one first RNA output.

45. The method of claim 44, wherein measuring a signal corresponding to the at least one first RNA output comprises measuring the second RNA output, the third RNA output and/or the reference RNA output.

46. The method of claims 44 or 45, wherein the incubating is done under conditions to promote transcription.

47. The method of claim 46, wherein the conditions to promote transcription comprises incubating the system and sample in a reaction mixture comprising any or all of: nucleoside triphosphates, buffers, cofactors, detergents, reducing agents, small molecule additives, and enzymatic additives.

48. The method of any of claims 44-47, wherein the sample is a biological sample.

49. The method of any of claims 44-48, wherein the molecule is a biomolecule.

50. The method of any of claims 44-49, wherein the molecule is a biomarker.

51. The method of any of claims 44-50, wherein the molecule is a protein.

52. The method of any of claims 44-51, wherein the system comprises two or more first transcription templates directed to two or more different molecules.

53. The method of any of claims 44-52, wherein the two or more different molecules are biomolecules.

54. The method of any of claims 44-53, wherein the method further comprises determining the presence or risk of a disease or disorder based on the RNA output.
